(19) United States
(12) Patent Application Publication
(10) Pub. No.: US 2024/0262844 A1
(43) Pub. Date: Aug. 8, 2024

(54) PYRIDINIUM SALTS AND METHODS OF USE

(71) Applicant: UNIVERSITY OF WYOMING, Laramie, WY (US)

(72) Inventors: Michael T. Taylor, Laramie, WY (US); Caleb R. Hoopes, Laramie, WY (US); Akash M. Sarkar, Laramie, WY (US); Nicholas J. Kuehl, Laramie, WY (US); Nicole L. Collins, Laramie, WY (US)

(21) Appl. No.: 18/099,760

(22) Filed: Jan. 20, 2023

(51) Int. Cl. C07D 495/04; C07D 213/89

(52) U.S. Cl. CPC C07D 495/04; C07D 213/89

(57) ABSTRACT

Embodiments of the present disclosure generally relate to pyridinium salts, compositions thereof, and to methods of use. In an embodiment, a pyridinium salt described herein includes a group that is transferred to a tryptophan residue or moiety of a tryptophan-containing molecule. In another embodiment, a method for modifying a tryptophan residue is provided. The method includes forming a mixture comprising a pyridinium salt and a molecule containing a tryptophan moiety, and exposing the mixture to light to chemically modify the tryptophan moiety of the molecule.

### GOVERNMENT RIGHTS

This invention was made with government support under CHE-2048201 and CHE-1429615 awarded by the National Science Foundation. The government has certain rights in the invention.

### BACKGROUND

### Field

Embodiments of the present disclosure generally relate to pyridinium salts, compositions thereof, and to methods of use.

### Description of the Related Art

Tryptophan plays a variety of critical functional roles in protein biochemistry. However, owing to, for example, tryptophan's low natural abundance and poor nucleophilicity of its indole side chain, the design of effective methods for both single protein bioconjugation at tryptophan as well as for in-situ chemoproteomic profiling remains a challenge. Conventional chemoproteomic profiling technologies make use of either direct or in-situ generated electrophiles that target either strongly nucleophilic species, such as cysteine, or higher abundance residues of moderate to weak nucleophilicity such as lysine, serine/threonine, tyrosine, and aspartate/glutamate. Other conventional technologies target weakly nucleophilic, low abundance methionine residues in lysates.

However, electrophilic approaches directed toward tryptophan remains a persistent challenge. The combination of tryptophan's low natural frequency and poor nucleophilicity necessitates that any tryptophan-selective electrophile would have to possess both sufficient kinetic competency and selectivity to label tryptophan (for example, in situ) in a complex proteome at low concentrations of reagent. As such, tryptophan has been the subject of many labeling approaches, including through the use of sulfenyl chlorides, metallocarbenoids, nitroxides, transition-metal-catalyzed processes, oxidative strategies, and photoredox catalysis. Such methods, however, require significant adaptation for use as chemical probes for tryptophan residues in situ.

One conventional approach utilizes a 2-nitrobenzenesulfenyl chloride isotopologue for chemoselective tryptophan modification in rat serum, resulting in the identification of three tryptic peptides harboring a tryptophan modification. Another conventional approach allows enrichment of tryptophan-containing peptides through reversible condensation of malondialdehyde onto the tryptophan-indolic N—H followed by enrichment on hydrazide-derivatized beads, resulting in the identification of nine proteins from yeast lysates. However, these conventional strategies require either pre-denaturation of proteins and labeling performance under acidic conditions or the use of unselective chemistries, precluding the use of these methods in more complex environments.

There is a need for new and improved compositions and methods for tryptophan modification.

### SUMMARY

Embodiments of the present disclosure generally relate to pyridinium salts, compositions thereof, and to methods of use. Unlike conventional technologies that require pre-denaturation of proteins, labeling under acidic conditions, the use of unselective chemistries, or the use of electrophilic reagents, embodiments described herein utilize pyridinium salts and light to chemically modify a tryptophan residue or moiety of a biological molecule, a biologically-derived molecule, or a synthetic molecule.

In an embodiment, a pyridinium salt described herein includes a group that is transferred to a tryptophan residue or moiety of a tryptophan-containing molecule.

In an embodiment a composition is provided. The composition includes a pyridinium salt represented by formula (I), an ion thereof, or combinations thereof:

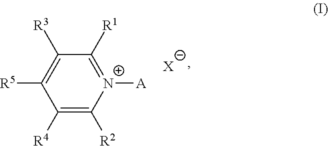

wherein:
A of formula (I) is an unsubstituted C1 to C40 hydrocarbyl, substituted C1 to C40 hydrocarbyl, or a functional group comprising at least one element of Group 13-17 of the periodic table of the elements;
each of R¹, R², R³, R⁴, and R⁵ of formula (I) is independently hydrogen, unsubstituted C1 to C40 hydrocarbyl, substituted C1 to C40 hydrocarbyl, or a functional group comprising at least one element of Group 13-17 of the periodic table of the elements; and
X⁻ of formula (I) is a monoatomic or polyatomic anion.

In another embodiment, a method for modifying a tryptophan moiety is provided. The method includes forming a mixture comprising a molecule containing a tryptophan moiety, and a composition comprising a pyridinium salt, an ion thereof, or a combination thereof. The method further includes exposing the mixture to light to chemically modify the tryptophan moiety of the molecule.

In another embodiment, a method of polypeptide modification is provided. The method includes irradiating a mixture comprising a pyridinium salt described herein and a polypeptide containing at least one tryptophan residue with ultraviolet light, visible light, or both to chemically modify the at least one tryptophan residue of the polypeptide.

In another embodiment, a composition is provided. The composition includes a pyridinium salt represented by formula (V), an ion thereof, or combinations thereof:

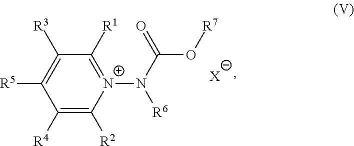

wherein:
each of R¹, R², R³, R⁴, R⁵, and R⁶ of formula (V) is independently hydrogen, unsubstituted C1 to C40 hydrocarbyl, substituted C1 to C40 hydrocarbyl, or a functional group comprising at least one element of Group 13-17 of the periodic table of the elements;
X⁻ of formula (V) is a monoatomic or polyatomic anion; and
R⁷ of formula (V) is an unsubstituted C1 to C40 hydrocarbyl or a substituted C1 to C40 hydrocarbyl; and
when (a) R⁶ is methyl, (b) X⁻ is BF₄⁻ or CF₃CO₂, (c) each of R¹, R², R³, and R⁴ are hydrogen, and (d) R⁵ is

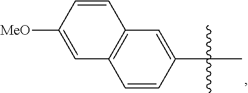 [SMILES: *c1ccc2cc(OC)ccc2c1]

then R⁷ is not —(CH₂)₆N₃ or

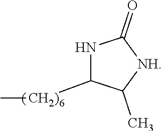

In another embodiment, a composition is provided. The composition includes a pyridinium salt represented by formula (I), an ion thereof, or combinations thereof:

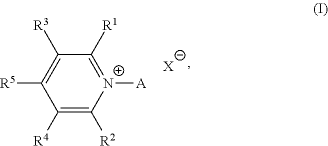

wherein:
each of R¹, R², R³, R⁴, and R⁵ of formula (I) is independently hydrogen, unsubstituted C1 to C10 hydrocarbyl, substituted C1 to C10 hydrocarbyl, or a functional group comprising at least one element of Group 13-17 of the periodic table of the elements;
X⁻ of formula (I) is selected from the group consisting of F⁻, Cl⁻, Br⁻, and I⁻, BF₄⁻, CF₃CO₂⁻, BCl₄⁻, BBr₄⁻, BI₄⁻, NO₃⁻, NO₂⁻, ClO₄⁻, IO₃⁻, ClO₃⁻, BrO₃⁻, ClO₂⁻, OCl⁻, OBr⁻, CN⁻, OCN⁻, SCN⁻, KMnO₄⁻, HSO₄⁻, HSO₃⁻, RSO₃⁻, H₂PO₄⁻, OH⁻, CH₃CO₂⁻, HCO₂⁻, HCO₃⁻, H₃CC₆H₄SO₂⁻, F₃CSO₃⁻, CH₃SO₃⁻, C₇H₅O₂⁻, C₃H₅O₃⁻, (CH₃(CH₂)₃)₂HPO₄⁻, and (C₆H₅)₄B⁻; and
A of formula (I) is selected from the group consisting of formula (IIIb) and formula (IIIc):

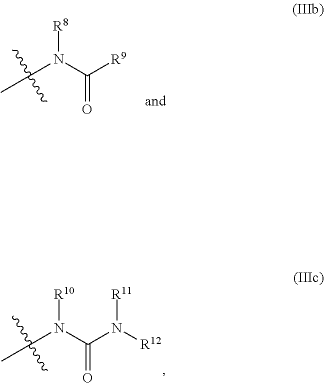

wherein:
each of R⁸ and R⁹ of formula (IIIb) is independently hydrogen, unsubstituted C1 to C20 hydrocarbyl, or substituted C1 to C20 hydrocarbyl; and
each of R¹⁰, R¹¹, and R¹² of formula (IIIc) is independently hydrogen, unsubstituted C1 to C20 hydrocarbyl, or substituted C1 to C20 hydrocarbyl.

In another embodiment, a method for modifying a tryptophan residue is provided. The method includes forming a mixture comprising a molecule containing a tryptophan moiety, and a composition comprising a pyridinium salt, an ion thereof, or a combination thereof. The method further includes exposing the mixture to light to chemically modify the tryptophan moiety of the molecule. The pyridinium salt is represented by formula (V):

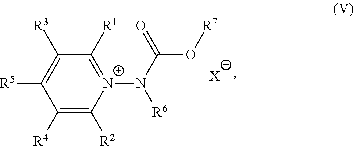

wherein:
each of R¹, R², R³, R⁴, R⁵, and R⁶ of formula (V) is independently hydrogen, unsubstituted C1 to C40 hydrocarbyl, substituted C1 to C40 hydrocarbyl, or a functional group comprising at least one element of Group 13-17 of the periodic table of the elements;
X⁻ of formula (V) is a monoatomic or polyatomic anion; and
R⁷ of formula (V) is an unsubstituted C1 to C40 hydrocarbyl or a substituted C1 to C40 hydrocarbyl; and
when (a) R⁶ is methyl, (b) X⁻ is BF₄⁻ or CF₃CO₂⁻, (c) each of R¹, R², R³, and R⁴ are hydrogen, and (d) R⁵ is

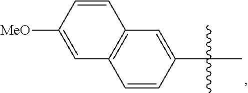 [SMILES: *c1ccc2cc(OC)ccc2c1]

then R⁷ is not —(CH₂)₆N₃ or

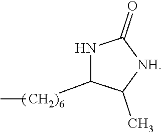

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 shows a non-limiting reaction diagram for covalently modifying tryptophan according to at least one embodiment of the present disclosure.

FIG. 2 shows a non-limiting reaction diagram of an example tryptophan modification according to at least one embodiment of the present disclosure.

FIG. 3 shows selected operations of a method of modifying tryptophan according to at least one embodiment of the present disclosure.

FIG. 4 shows a non-limiting reaction diagram for forming a pyridinium salt according to at least one embodiment of the present disclosure.

FIG. 5 shows selected operations of a method of making pyridinium salt according to at least one embodiment of the present disclosure.

FIG. 6 is a plot of conversion versus time and temporal control of the labeling of lysozyme with an example pyridinium salt according to at least one embodiment of the present disclosure.

FIG. 7 is a Stern-Volmer plot of florescence quenching of an example pyridinium salt with a small molecule tryptophan analog N-acetyltryptophanamide (NATA) according to at least one embodiment of the present disclosure.

FIG. 8A shows absorption spectra of an example pyridinium salt in selected solvents according to at least one embodiment of the present disclosure.

FIG. 8B shows emission spectra of an example pyridinium salt in selected organic solvents according to at least one embodiment of the present disclosure.

FIG. 8C shows emission spectra of an example pyridinium salt in CH₃CN or H₂O according to at least one embodiment of the present disclosure.

FIG. 8D shows emission spectra of two example pyridinium salts according to at least one embodiment of the present disclosure.

FIG. 9A is a fluorescence decay spectrum of an example pyridinium salt in 1,2-dichloroethane according to at least one embodiment of the present disclosure.

FIG. 9B is a fluorescence decay spectrum of an example pyridinium salt in acetonitrile according to at least one embodiment of the present disclosure

FIG. 9C is a fluorescence decay spectrum of an example pyridinium salt in water according to at least one embodiment of the present disclosure.

FIG. 9D is a fluorescence decay spectrum of another example pyridinium salt in water according to at least one embodiment of the present disclosure.

FIG. 10 is a cyclic voltammogram of an example pyridinium salt in acetonitrile according to at least one embodiment of the present disclosure.

FIG. 11 shows an image of an SDS-Page gel of a purified nucleophosmin 1 (NPM1) [240-294] peptide construct that was used for labeling/modification experiments according to at least one embodiment of the present disclosure.

FIG. 12 shows an image of a Western blot validating the presence of NPM1 in enriched proteins from in-situ chemoproteomic profiling of HEK293T cells using an example pyridinium salt according to at least one embodiment of the present disclosure.

FIG. 13 shows an image of a Western blot validating the presence of Poly [ADP-ribose] polymerase 1 (PARP1) in enriched proteins from in-situ chemoproteomic profiling of HEK293T cells using an example pyridinium salt according to at least one embodiment of the present disclosure.

FIG. 14 shows an image of a Western blot of the labeling of a recombinant NPM1 C-terminal domain using an example pyridinium salt according to at least one embodiment of the present disclosure.

FIGS. 15A-15C show Western blots of elution profiles of chemoproteomic profiling of HEKT293T lysates with three example pyridinium salts according to at least one embodiment of the present disclosure.

FIG. 16 is a scatter plot of the solvent accessibility of tryptophan residues of proteins investigated herein and whether the tryptophan residue is labeled.

FIG. 17A is a bar chart showing the number of proteins identified at 100 μM of example pyridinium salts according to at least one embodiment of the present disclosure.

FIG. 17B is a Venn diagram comparing overlap of proteome coverage by example pyridinium salts according to at least one embodiment of the present disclosure.

FIG. 17C is classes of proteins showing significant enrichment relative to all detected proteins with 100 μM of an example pyridinium salt according to at least one embodiment of the present disclosure (the −log₁₀ p-values are shown to clearly highlight enrichment).

FIG. 17D shows the light dependence of protein-level enrichment with an example pyridinium salt according to at least one embodiment of the present disclosure.

FIGS. 18A-18C are volcano plots showing light-dependent and dose-responsive enrichment of the tryptophan-ome according to at least one embodiment of the present disclosure (average of two experiments).

FIG. 19A is a bar chart showing detected residue modifications by percentage according to at least one embodiment of the present disclosure.

FIG. 19B is a pie chart showing chemoselectivity based upon amino acid relative frequency according to at least one embodiment of the present disclosure.

FIG. 20 shows a comparison of solvent accessibility in modified and unmodified tryptophan residues of proteins identified in situ with an example pyridinium salt according to at least one embodiment of the present disclosure.

FIG. 21 shows validation of the identification of NPM1 and PARP1 via western analysis of post-protein-level enrichment profiles of HEK293T cells labeled with an example pyridinium salt according to at least one embodiment of the present disclosure.

Figures included herein illustrate various embodiments of the disclosure. It is contemplated that elements and features of one embodiment may be beneficially incorporated in other embodiments without further recitation.

### DETAILED DESCRIPTION

Embodiments of the present disclosure generally relate to pyridinium salts, compositions thereof, and to methods of use. The inventors have found compositions and methods for tryptophan modification. In general, the compositions and methods described herein can circumvent tryptophan's weak nucleophilicity by utilizing photo-induced electron transfer (PET). Here, for example, a tryptophan moiety present in a biological molecule can be chemically modified by the use of a pyridinium salt reagent in the presence of light. The pyridinium salts contain a group that is transferred to the tryptophan moiety present in the biological molecule. The pyridinium salts described herein are thermally stable, photo-stable, and compatible with aqueous environments and biological antioxidants. Unlike conventional compositions and methods for modifying biological molecules, embodiments of the present disclosure show good selectivity for tryptophan and short reaction times.

Embodiments described herein also relate to chemoproteomic profiling where the pyridinium salt serves as a probe. For example, and in some embodiments, pyridinium salts described herein can be utilized to modify tryptophan-containing peptides. Spatiotemporal control enabled by, for example, optical/light triggering, permits control over aspects such as probe incubation time, allowing for sufficient time for cellular penetration and compartmentalization. Chemoproteomic profiling using pyridinium salts as described herein can enable, for example, the discovery of new tryptophan residues, sites of non-covalent interactions, and biomolecular interfaces. This, in turn, enables their consideration as, for example, ligandable sites for drug discovery.

Tryptophan plays a variety of critical functional roles in protein biochemistry. Tryptophan is widely dispersed throughout the proteome and is also relatively evenly distributed between surface exposed and buried positions. Tryptophan possesses the most electron-rich π-system of the naturally occurring amino acids, a property that enables tryptophan to engage in electrostatically driven non-covalent interactions such as π-π interactions, cation-x interactions, and X-H-π interactions (e.g., C—H-π, O—H-π, among others) as well as through hydrogen bonding (H-bonding) to neighboring functionality via the indolic N—H bond. As a result, tryptophan can be found enriched at centers of biochemical significance such as protein-protein interfaces and protein-lipid interfaces (2.9% abundance in membrane proteins), as well as serving to maintain protein structural integrity through intraprotein non-covalent interactions. Owing to tryptophan's electron-rich π-system, further enrichment of tryptophan can be observed in the redox proteome.

Recently, site-selective protein modification technologies have been adapted for use in broad spectrum chemoproteomic interrogation of a given reactive moiety (typically, an amino acid side chain). This technology can enable the characterization of reactive hotspots surrounding the moiety of interest. Such information can then lead to the discovery of new sites of biological significance and ultimately the discovery of new druggable sites. Conventional chemoproteomic profiling strategies make use of either direct or in-situ generated electrophiles that target either strongly nucleophilic species, such as cysteine, or higher abundance residues of moderate to weak nucleophilicity such as lysine, serine/threonine, tyrosine, and aspartate/glutamate. Other conventional technologies target weakly nucleophilic, low abundance methionine residues in lysates.

However, electrophilic approaches directed toward other functionally important residues, such as tryptophan, remain a persistent challenge. The challenge of chemoproteomic profiling of tryptophan stems from, e.g., a combination of tryptophan's very low natural abundance and the modest nucleophilicity of its indole side chain. Conventional technologies for chemoproteomics and labeling rely on the use of sulfenyl chlorides, metallocarbenoids, nitroxides, transition-metal-catalyzed processes, oxidative strategies, and photoredox catalysis. Such methods, however, require significant adaptation for use as chemical probes for tryptophan residues in situ.

2-nitrobenzenesulfenyl chloride isotopologues have been utilized for tryptophan modification in rat serum, and a sequence of malondialdehyde-indole condensation followed by enrichment on hydrazide-derivatized beads has resulted in the identification of nine proteins from yeast lysates. However, these conventional strategies require either pre-denaturation of proteins and labeling performance under acidic conditions or the use of unselective chemistries, precluding the use of these methods in more complex environments.

In contrast to these conventional technologies, embodiments described herein do not require pre-denaturation of proteins, labeling under acidic conditions, or the use of unselective chemistries typical of conventional technologies for modifying biological molecules.

The use of headings is for purposes of convenience only and does not limit the scope of the present disclosure. Embodiments described herein can be combined with other embodiments.

As used herein, a “composition” can include component(s) of the composition, reaction product(s) of two or more components of the composition, a remainder balance of remaining starting component(s), or combinations thereof. Compositions of the present disclosure can be prepared by any suitable mixing process.

### Compositions

Embodiments described herein relate to compositions comprising, consisting essentially of, or consisting of a pyridinium salt, an ion thereof, or combinations thereof. The compositions can be utilized for, e.g., chemically modifying a tryptophan residue or moiety of a biological molecule, a biologically-derived molecule, or a synthetic molecule. The compositions can be used for chemoproteomics such as chemoproteomic profiling. In addition, the compositions can be utilized to identify and interrogate protein-small molecule interactions, drug discovery, modifying a target protein, validation of a molecule's mechanism of action. Other applications are contemplated.

Compositions described herein comprise, consist essentially of, or consist of a pyridinium salt represented by formula (I), an ion thereof, or combinations thereof:

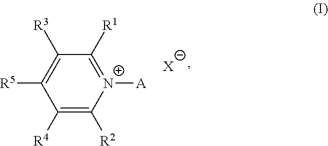

wherein:
each of R¹, R², R³, R⁴, and R⁵ is independently hydrogen, unsubstituted hydrocarbyl (such as C1 to C40 unsubstituted hydrocarbyl), substituted hydrocarbyl (such as C1 to C40 substituted hydrocarbyl), or a functional group comprising at least one element of Group 13-17 of the periodic table of the elements;
A is an unsubstituted hydrocarbyl (such as a C1 to C40 unsubstituted hydrocarbyl), a substituted hydrocarbyl (such as a C1 to C40 substituted hydrocarbyl), or a functional group comprising at least one element of Group 13-17 of the periodic table of the elements; and
X⁻ is a monoatomic or polyatomic anion.

In formula (I), one or more of R¹, R², R³, R⁴, or R⁵ can have, independently, any suitable number of carbon atoms such as from 1 to 40 carbon atoms, such as from 1 to 20 carbon atoms, such as from 1 to 12 carbon atoms, such as from 1 to 10 carbon atoms, such as from 1 to 8 carbon atoms, such as from 1 to 5 carbon atoms, 1 to 4 carbon atoms, or from 3 to 8 carbon atoms. In some embodiments, the number of carbon atoms in one or more of R¹, R², R³, R⁴, or R⁵ of formula (I) can be, independently, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or ranges thereof, though other numbers of carbon atoms are contemplated. Each of the foregoing numbers can be preceded by the word “about,” “at least about,” “less than about,” or “more than about,” and any of the foregoing numbers can be used singly to describe an open-ended range or in combination to describe a close-ended range. One or more of R¹, R², R³, R⁴, or R⁵ of formula (I) can be, independently, linear or branched, saturated or unsaturated, cyclic or acyclic, monocyclic or polycyclic, aromatic or not aromatic. Regarding saturation, one or more of R¹, R², R³, R⁴, or R⁵ of formula (I) can be, independently, fully saturated, partially unsaturated, or fully unsaturated.

An “unsubstituted hydrocarbyl” refers to a group that consists of hydrogen and carbon atoms only. Non-limiting examples of unsubstituted hydrocarbyl include an alkyl group having from 1 to 40 carbon atoms (such as from 1 to 20 carbon atoms) such as methyl, ethyl, n-propyl, isopropyl, n-butyl, iso-butyl, sec-butyl, and tert-butyl, pentyl, hexyl, heptyl, octyl, ethyl-2-hexyl, isooctyl, nonyl, n-decyl, isodecyl, or isomers thereof; a cycloaliphatic group having from 3 to 40 carbon atoms, such as from 3 to 20 carbon atoms such as, for example, cyclopentyl or cyclohexyl; an aryl group having from 5 to 40 carbon atoms, such as from 6 to 20 carbon atoms such as, for example, phenyl, naphthyl, fluorenyl, or xylyl; or any combination thereof. Any of the foregoing numbers can be used singly to describe an open-ended range or in combination to describe a close-ended range.

The term “aryl” or “aryl group” refers to an aromatic ring such as phenyl, naphthyl, xylyl, etc. Likewise, heteroaryl refers to an aryl group where a ring carbon atom (or two or three ring carbon atoms) has been replaced with a heteroatom, such as N, O, or S. As used herein, the term “aromatic” also refers to pseudoaromatic heterocycles which are heterocyclic substituents that have similar properties and structures (nearly planar) to aromatic heterocyclic ligands, but are not by definition aromatic. A heterocyclic ring is a ring having a heteroatom in the ring structure as opposed to a heteroatom substituted ring where a hydrogen on a ring atom is replaced with a heteroatom. For example, tetrahydrofuran is a heterocyclic ring and 4-N,N-dimethylamino-phenyl is a heteroatom substituted ring.

A “substituted hydrocarbyl” refers to an unsubstituted hydrocarbyl in which at least one hydrogen of the unsubstituted hydrocarbyl has been substituted with at least one heteroatom or heteroatom-containing group, such as one or more elements from Group 13-17 of the periodic table of the elements, such as halogen (F, Cl, Br, or I), O, N, Se, Te, P, As, Sb, S, B, Si, Ge, Sn, Pb, and the like, such as C(O)R*, C(C)NR*₂, C(O)OR*, NR*₂, OR*, SeR*, TeR*, PR*₂, AsR*₂, SbR*₂, SR*, SOₓ (where x=2 or 3), BR*₂, SiR*₃, GeR*₃, SnR*₃, PbR*₃, and the like, where R* is, independently, hydrogen or unsubstituted hydrocarbyl, or where at least one heteroatom has been inserted within the unsubstituted hydrocarbyl. The substituted hydrocarbyl can be a heteroaryl group such as a tetrahydrofuran.

As described above, one or more of R¹, R², R³, or R⁴ of formula (I) can be, independently, aromatic. In some embodiments, one or more of R¹, R², R³, or R⁴ of formula (I) can be, independently, an unsubstituted aryl (such as such as unsubstituted phenyl, unsubstituted naphthyl, unsubstituted fluorenyl), a substituted aryl (such as alkylphenyl, alkoxyphenyl, halogenated phenyl, alkoxynaphthyl, alkylnaphthyl, a halogenated naphthyl), an unsubstituted heteroaryl (such as pyrrole, imidazole, pyrazole, thiophene, furan), a substituted heteroaryl, among others. When one or more of R¹, R², R³, or R⁴ of formula (I) is an unsubstituted aryl, a substituted aryl, an unsubstituted heteroaryl, or a substituted heteroaryl, each of the unsubstituted aryl, the substituted aryl, the unsubstituted heteroaryl, and the substituted heteroaryl can, independently, have from 4 to 40 carbon atoms, such as from 5 to 20 carbon atoms, such as from 6 to 12 carbon atoms, such as from 6 to 10 carbon atoms.

In some embodiments, one or more of R¹, R², R³, or R⁴ of formula (I) can be, independently, formula (IIa) or (IIb) as described below.

As described above, one or more of R¹, R², R³, R⁴, or R⁵ of formula (I) can be a functional group comprising at least one element of Group 13-17 of the periodic table of the elements. Such functional groups can include halogen (F, Cl, Br, or I), O, N, Se, Te, P, As, Sb, S, B, Si, Ge, Sn, Pb, and the like, such as C(O)R*, C(C)NR*₂, C(O)OR*, NR*₂, OR*, SeR*, TeR*, PR*₂, AsR*₂, SbR*₂, SR*, SOₓ (where x=2 or 3), BR*₂, SiR*₃, GeR*₃, SnR*₃, PbR*₃, and the like, where R* is, independently, hydrogen or unsubstituted hydrocarbyl, or where at least one heteroatom has been inserted within the unsubstituted hydrocarbyl.

In some embodiments, one or more of R¹, R², R³, R⁴, or R⁵ of formula (I) can be or include, independently, an alkoxy group. An “alkoxy” refers to —O— alkyl group containing from 1 to 20 carbon atoms, such as from 1 to 10 carbon atoms. The alkoxy may be straight-chain or branched-chain. Non-limiting examples include methoxy, ethoxy, propoxy, butoxy, isobutoxy, tert-butoxy, pentoxy, and hexoxy. “C₁ alkoxy” refers to methoxy, “C₂ alkoxy” refers to ethoxy, “C₃ alkoxy” refers to propoxy and “C₄ alkoxy” refers to butoxy. Further, as used herein, “OMe” refers to methoxy and “OEt” refers to ethoxy.

In at least one embodiment, one or more of R¹, R², R³, or R⁴ of formula (I) can be, independently, hydrogen, a C1 to C10 alkyl (such as C1 to C6 alkyl, such as C to C4 alkyl, such as methyl, ethyl, propyl, or butyl), or a C1 to C10 alkoxy (such as C1 to C6 alkoxy, such as C1 to C4 alkoxy, such as methoxy, ethoxy, propoxy, or butoxy).

In some embodiments, R⁵ of formula (I) can be an unsubstituted aryl (such as such as unsubstituted phenyl, unsubstituted naphthyl, unsubstituted fluorenyl), a substituted aryl (such as alkylphenyl, alkoxyphenyl, halogenated phenyl, alkoxynaphthyl, alkylnaphthyl, a halogenated naphthyl), an unsubstituted heteroaryl (such as pyrrole, imidazole, pyrazole, thiophene, furan), a substituted heteroaryl, among others. When R⁵ of formula (I) is an unsubstituted aryl, a substituted aryl, an unsubstituted heteroaryl, or a substituted heteroaryl, each of the unsubstituted aryl, the substituted aryl, the unsubstituted heteroaryl, and the substituted heteroaryl can, independently, have from 4 to 40 carbon atoms, such as from 5 to 20 carbon atoms, such as from 6 to 12 carbon atoms, such as from 6 to 10 carbon atoms.

R⁵ of formula (I) can be represented by formula (IIa):

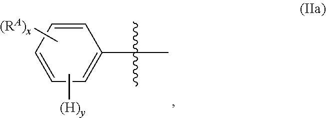

wherein, in formula (IIa):
the wavy bond represents the connection to the pyridine ring;
H is a hydrogen atom on the aromatic ring;
R^{A} is an unsubstituted hydrocarbyl having from 1 to 20 carbon atoms, a substituted hydrocarbyl having from 1 to 20 carbon atoms, or a functional group comprising at least one element of Group 13-17 of the periodic table of the elements;
x is from 0 to 5, such as 0, 1, 2, 3, 4, or 5, and when x is more than 1, each R^{A} group is the same or different;
y is from 0 to 5, such as 0, 1, 2, 3, 4, or 5; and
x+y is 5.

Illustrative, but non-limiting, examples of each R^{A} group of formula (IIa) can include one or more of those groups described above for R¹ of formula (I). For example, each R^{A} group c of formula (IIa) an be, independently, an unsubstituted hydrocarbyl having from 1 to 4 carbon atoms or an alkoxy group having from 1 to 4 carbon atoms, such as methyl, ethyl, methoxy, or ethoxy, among others.

In some embodiments, formula (IIa) is:

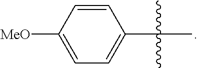 [SMILES: *c1ccc(OC)cc1]

R⁵ of formula (I) can be represented by formula (IIb):

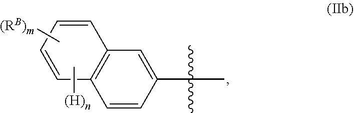

wherein, in formula (IIb):
the wavy bond represents the connection to the pyridine ring;
H is a hydrogen atom on the naphthyl ring;
R^{B} is an unsubstituted hydrocarbyl having from 1 to 20 carbon atoms, a substituted hydrocarbyl having from 1 to 20 carbon atoms, or a functional group comprising at least one element of Group 13-17 of the periodic table of the elements;
m is from 0 to 7, such as 0, 1, 2, 3, 4, 5, 6, or 7, and when m is more than 1, each R^{B} group is the same or different;
n is from 0 to 7, such as 0, 1, 2, 3, 4, 5, 6, or 7; and
m+n is 7.

Illustrative, but non-limiting, examples of each R^{B} group of formula (IIb) can include one or more of those groups described above for R¹ of formula (I). For example, each R^{B} group of formula (IIb) can be, independently, an unsubstituted hydrocarbyl having from 1 to 4 carbon atoms or an alkoxy group having from 1 to 4 carbon atoms, such as methyl, ethyl, methoxy, or ethoxy, among others.

In some embodiments, formula (IIb) is:

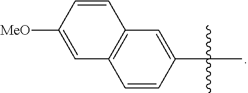 [SMILES: *c1ccc2cc(OC)ccc2c1]

Referring back to formula (I), and in some embodiments, the A group of the pyridinium salt is transferred to a tryptophan residue or moiety of a tryptophan-containing molecule. Accordingly, and in some embodiments, the A group of formula (I) can be used to install functional handles useful for, e.g., chemoproteomics and general bioconjugation strategies onto larger molecules such as proteins. Other applications are contemplated.

As described above, the A group can an unsubstituted hydrocarbyl (such as a C1 to C40 unsubstituted hydrocarbyl), a substituted hydrocarbyl (such as a C1 to C40 substituted hydrocarbyl), or a functional group comprising at least one element of Group 13-17 of the periodic table of the elements

When the A group of formula (I) is a functional group comprising at least one element from Group 13-17, the A group can be halogen (F, Cl, Br, or I), O, N, Se, Te, P, As, Sb, S, B, Si, Ge, Sn, Pb, and the like, such as NR*₂, C(O)R*, C(C)NR*₂, C(O)OR*, OR*, SeR*, TeR*, PR*₂, AsR*₂, SbR*₂, SR*, SOₓ (where x=2 or 3), BR*₂, SiR*₃, GeR*₃, SnR*₃, PbR*₃, and the like, where R* can be, independently, hydrogen or unsubstituted hydrocarbyl, or where at least one heteroatom has been inserted within the unsubstituted hydrocarbyl.

The A group of formula (I) can have any suitable number of carbon atoms such as from 1 to 40 carbon atoms, such as from 1 to 20 carbon atoms, such as from 1 to 12 carbon atoms, such as from 1 to 10 carbon atoms, such as from 1 to 8 carbon atoms, such as from 1 to 5 carbon atoms, 1 to 4 carbon atoms, or from 3 to 8 carbon atoms. In some embodiments, the number of carbon atoms of the A group of formula (I) can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or ranges thereof, though other numbers of carbon atoms are contemplated. Each of the foregoing numbers can be preceded by the word “about,” “at least about,” “less than about,” or “more than about,” and any of the foregoing numbers can be used singly to describe an open-ended range or in combination to describe a close-ended range. The A group of formula (I) can be linear or branched, saturated or unsaturated, cyclic or acyclic, monocyclic or polycyclic, aromatic or not aromatic. Regarding saturation, the A group of formula (I) can be fully saturated, partially unsaturated, or fully unsaturated.

In some examples, the A group of formula (I) can be a carbamate group of formula (IIIa), an amide group of formula (IIIb), or a urea group of formula (IIIc), among other possibilities:

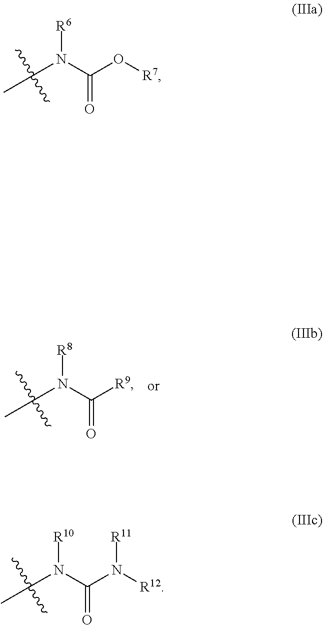

In formulas (IIIa)-(IIIc), the wavy represents the connection to the nitrogen atom of the pyridine ring. In formulas (IIIa)-(IIIc), each of R⁶, R⁷, R⁸, R⁹, R¹⁰, R¹¹, and R¹² is, independently, hydrogen, unsubstituted hydrocarbyl, substituted hydrocarbyl, or a functional group comprising at least one element from Group 13-17. When one or more of R⁶, R⁷, R⁸, R⁹, R¹⁰, R¹¹, or R¹² is a functional group comprising at least one element from Group 13-17, one or more of R⁶, R⁷, R⁸, R⁹, R¹⁰, R¹¹, or R¹² can be, independently, halogen (F, Cl, Br, or I), O, N, Se, Te, P, As, Sb, S, B, Si, Ge, Sn, Pb, and the like, such as NR*₂, C(O)R*, C(C)NR*₂, C(O)OR*, OR*, SeR*, TeR*, PR*₂, AsR*₂, SbR*₂, SR*, SOₓ (where x=2 or 3), BR*₂, SiR*₃, GeR*₃, SnR*₃, PbR*₃, and the like, where R* can be, independently, hydrogen or unsubstituted hydrocarbyl, or where at least one heteroatom has been inserted within the unsubstituted hydrocarbyl.

One or more of R⁶, R⁷, R⁸, R⁹, R¹⁰, R¹¹, or R¹² of formulas (IIIa)-(IIIc) can have, independently, any suitable number of carbon atoms such as from 1 to 40 carbon atoms, such as from 1 to 20 carbon atoms, such as from 1 to 12 carbon atoms, such as from 1 to 10 carbon atoms, such as from 1 to 8 carbon atoms, such as from 1 to 5 carbon atoms, 1 to 4 carbon atoms, or from 3 to 8 carbon atoms. In some embodiments, the number of carbon atoms can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or ranges thereof, though other numbers of carbon atoms are contemplated. Each of the foregoing numbers can be preceded by the word “about,” “at least about,” “less than about,” or “more than about,” and any of the foregoing numbers can be used singly to describe an open-ended range or in combination to describe a close-ended range. One or more of R⁶, R⁷, R⁸, R⁹, R¹⁰, R¹¹, or R¹² can be, independently, linear or branched, saturated or unsaturated, cyclic or acyclic, monocyclic or polycyclic, aromatic or not aromatic. Regarding saturation, one or more of R⁶, R⁷, R⁸, R⁹, R¹⁰, R¹¹, or R¹² can be, independently, fully saturated, partially unsaturated, or fully unsaturated.

In at least one embodiment, one or more of R⁶, R⁷, R⁸, R⁹, R¹⁰, R¹¹, or R¹² of formulas (IIIa)-(IIIc) can be, independently, an alkyl group having from 1 to 40 carbon atoms (such as from 1 to 20 carbon atoms) such as methyl, ethyl, n-propyl, isopropyl, n-butyl, iso-butyl, sec-butyl, and tert-butyl, pentyl, hexyl, heptyl, octyl, ethyl-2-hexyl, isooctyl, nonyl, n-decyl, isodecyl, or isomers thereof; a cycloaliphatic group having from 3 to 40 carbon atoms, such as from 3 to 20 carbon atoms such as, for example, cyclopentyl or cyclohexyl; an aryl group having from 5 to 40 carbon atoms, such as from 6 to 20 carbon atoms such as, for example, phenyl, naphthyl, fluorenyl; or any combination thereof. Any of the foregoing numbers can be used singly to describe an open-ended range or in combination to describe a close-ended range.

In some embodiments, one or more of R⁶, R⁷, R⁸, R⁹, R¹⁰, R¹¹, or R¹² of formulas (IIIa)-(IIIc) can be, independently, an unsubstituted aryl (such as such as unsubstituted phenyl, unsubstituted naphthyl, unsubstituted fluorenyl), a substituted aryl (such as alkylphenyl, alkoxyphenyl, halogenated phenyl, alkoxynaphthyl, alkylnaphthyl, a halogenated naphthyl), an unsubstituted heteroaryl (such as pyrrole, imidazole, pyrazole, thiophene, furan), a substituted heteroaryl, among others. When one or more of R⁶, R⁷, R⁸, R⁹, R¹⁰, R¹¹, or R¹² of formulas (IIIa)-(IIIc) is an unsubstituted aryl, a substituted aryl, an unsubstituted heteroaryl, or a substituted heteroaryl, each of the unsubstituted aryl, the substituted aryl, the unsubstituted heteroaryl, and the substituted heteroaryl can, independently, have from 4 to 40 carbon atoms, such as from 5 to 20 carbon atoms, such as from 6 to 12 carbon atoms, such as from 6 to 10 carbon atoms. In some embodiments, one or more of R⁶, R⁷, R⁸, R⁹, R¹⁰, R¹¹, or R¹² of formulas (IIIa)-(IIIc) can be, independently, formula (IIa) or (IIb) as described above.

As described above, the anion (X⁻) of formula (I) is monoatomic or polyatomic. X⁻ of formula (I) can be any suitable anion. Illustrative, but non-limiting, examples of a monoatomic anion X⁻ can include a halide (F⁻, Cl⁻, Br⁻, and I⁻), an oxide, or combinations thereof. When the anion X⁻ is a polyatomic anion, X⁻ can be an inorganic anion, an organic anion, or combinations thereof. Illustrative, but non-limiting, examples of a polyatomic anion X⁻ can include tetrafluoroborate (BF₄⁻), trifluoroacetate (TFA, CF₃CO₂⁻), hexafluorophosphate (PF₆⁻), tetrachloroborate (BCl₄⁻), tetrabromoborate (BBr₄⁻), tetraiodoborate (BI₄⁻), nitrate (NO₃⁻), nitrite (NO₂⁻), perchlorate (ClO₄⁻), iodate (IO₃⁻), chlorate (ClO₃⁻), bromate (BrO₃⁻), chlorite (ClO₂⁻), hypochlorite (OCl⁻), hypobromite (OBr⁻), cyanide (CN⁻), cyanate (OCN⁻), thiocyanate (SCN⁻), permanganate (KMnO₄⁻), hydrogensulfate (HSO₄⁻), hydrogensulfite (HSO₃⁻), sulfonate (R**SO₃⁻), dihydrogen phosphate (H₂PO₄⁻), hydroxide (OH⁻), acetate (CH₃CO₂⁻), formate (HCO₂⁻), hydrogen carbonate (HCO₃⁻), toluenesulfonyl (tosyl, H₃CC₆H₄SO₂⁻), trifluoromethansulfonate (triflate, F₃CSO₃⁻), mesylate (CH₃SO₃⁻), benzoate (BzO, C₇H₅O₂⁻), lactate (C₃H₅O₃⁻), a phosphate ((R**PO₄⁻) such as dibutyl phosphate, (CH₃(CH₂)₃)₂HPO₄⁻)), tetraphenylborate ((C₆H₅)₄B⁻), or combinations thereof. Such anions can be a portion of a larger anion. For example, the sulfonate can have an R** group where the R** is any suitable organic group such as those R groups described herein. Other ions are contemplated.

In some embodiments, X⁻ comprises or is selected from the group consisting of F⁻, Cl⁻, Br⁻, I⁻, BF₄⁻, CF₃CO₂⁻, PF₆⁻, H₃CC₆H₄SO₂⁻ (tosyl), SCN⁻, BCl₄⁻, BBr₄⁻, BI₄⁻, F₃CSO₃⁻, CH₃CO₂⁻, CH₃SO₃⁻, C₇H₅O₂⁻, C₃H₅O₃⁻, (CH₃(CH₂)₃)₂HPO₄⁻, (C₆H₅)₄B⁻, and combinations thereof, such as BF₄⁻, CF₃CO₂⁻, and combinations thereof.

In a solution or suspension, the pyridinium salt(s) may exist as one or more ions. For example, one or more anions (for example, BF₄⁻, CF₃CO₂⁻, Cl⁻, Br, et cetera) and one or more cations (for example, pyridinium cation) may exist in the solution or suspension.

In some embodiments, the pyridinium salt of formula (I) can be represented by formula (IVa), formula (IVb), formula (IVc), formula (IVd), formula (IVe), or formula (IVf):

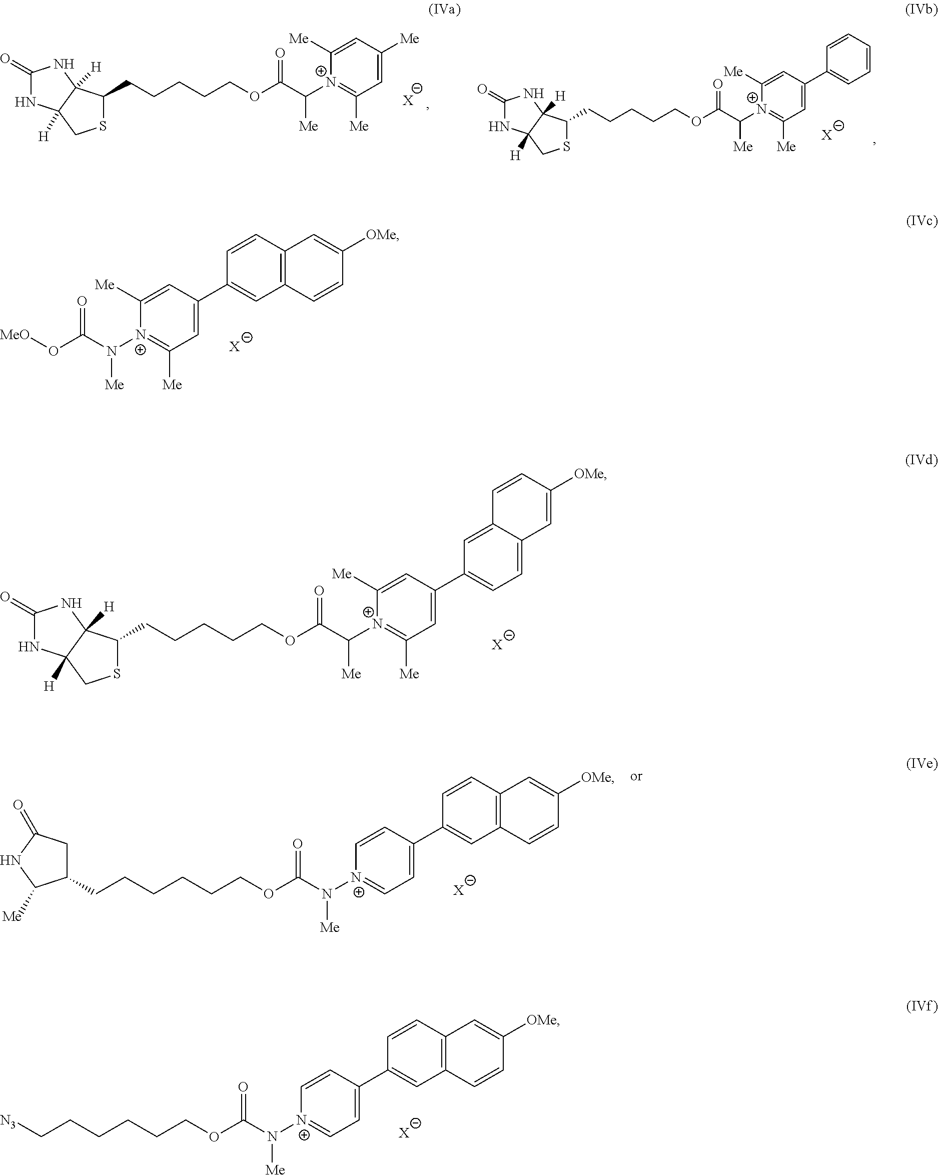

wherein, in formulas (IVa)-(IVf), X⁻ can be any suitable anion, such as those anions described for formula (I).

In some embodiments, the pyridinium salt of formula (I) is represented by the pyridinium salt of formula (V):

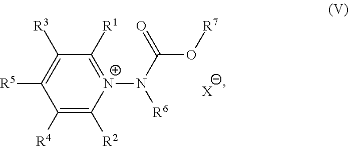

wherein each of X⁻, R¹, R², R³, R⁴, R⁵, R⁶, and R⁷ of formula (V) are described above with respect to formula (I) and formula (IIIa).

In some embodiments of formula (V), R⁷ of formula (V) is not —(CH₂)₆N₃ or

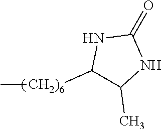

when:
(a) R⁶ of formula (V) is methyl (CH₃),
(b) X⁻ of formula (V) is BF₄⁻ or CF₃CO₂⁻,
(c) each of R¹, R², R³, and R⁴ of formula (V) are hydrogen, and
(d) R⁵ of formula (V) is

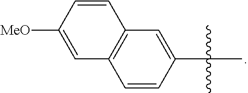 [SMILES: *c1ccc2cc(OC)ccc2c1]

Accordingly, and in some embodiments of formula (V), R⁷ is not 4-hexyl-5-methylimidazolidin-2-one and/or is not 1-azidohexane. In such embodiments of formula (V), the pyridinium salt is free of (or does not include) both the pyridinium salt of formula (IVe) wherein X⁻ is BF₄⁻ and the pyridinium salt of formula (IVf) wherein X⁻ is BF₄.

In some embodiments, R⁷ of formula (V) can be a substituted C1 to C40 hydrocarbyl comprising a sulfur atom, for example, the pyridinium salts of formula (IVa), formula (IVb), and formula (IVc), among other pyridinium salts.

Pyridinium salts described herein may exist as rotamers. The mixture of rotamers may have a molar ratio of about 1:1 to about 1.5:1, such as from about 1.1:1 to about 1.4:1, such as from about 1.2:1 to about 1.3:1. In at least one embodiment, a molar ratio of the rotamers can be 1:1, 1.1:1, 1.2:1, 1.3:1, 1.4:1, or 1.5:1, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range.

Pyridinium salts described herein can be made by any suitable method, such as those further described below.

### Methods of Using

Embodiments of the present disclosure also relate to methods of using pyridinium salts or compositions thereof. As described above, the pyridinium salts can be utilized to covalently modify, for example, an indole ring of a tryptophan in the presence of light such as visible light and/or UV light.

The tryptophan that is modified can be present as a peptide (a single amino acid), present as a portion of compound, present in a biological molecule (such as a polypeptide, a protein, or a proteome), present in a biologically-derived molecule, present in a synthetic molecule, among others. Polypeptides are two or more amino acids joined together by peptide bonds, and proteins contain one or more polypeptides. As used herein, these term “polypeptide” and “protein” are used interchangeably such that reference to one includes reference to both. For example, “polypeptide” includes reference to both “polypeptide” and “protein.” The peptide or polypeptide containing a tryptophan moiety (or residue) can be in a purified state, an unpurified state, a lysate, a proteome, in situ, in vitro, among other environments. The tryptophan-containing polypeptide, protein, or proteome can be present in live cells. The tryptophan can be surface exposed.

Although embodiments are described with respect to modifying a tryptophan of a biological molecule, embodiments described herein are applicable to biologically-derived molecules (e.g., at least partially synthesized) and synthetic molecules such as synthetic constructs, synthetic polypeptides, among others.

Embodiments described herein enable the tryptophan moiety/residue of the biological molecule (or other molecule) to be modified at the 2-position of the indole ring:

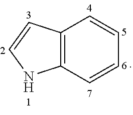

For biological molecules such as polypeptides and proteins, the indole ring of the tryptophan is bonded to the polypeptide or protein at the 3-position of the indole ring.

Any suitable biological molecule containing at least one tryptophan can be utilized with embodiments described herein. The biological molecule (such as a polypeptide) can be of any suitable size or weight. In some non-limiting examples, a weight of the biological molecule can be about 0.3 kilodaltons (kDa) or more or about 100 kDa or less, such as from about 1 kDa to about 75 kDa, such as from about 5 kDa to about 50 kDa, such as from about 10 kDa to about 25 kDa. In some embodiments, a weight (kDa) of the biological molecule that can be used for embodiments described herein can be 0.3, 0.5, 0.7, 0.9, 1, 1.2, 1.4, 1.6, 1.8, 2, 2.5, 3, 3.5, 4, 4.2, 4.5, 5, 6, 7, 8, 9, 10, 11, 11.6, 11.7, 12, 13, 14, 15, 16, 17, 18, 19, 20, 21, 22, 23, 24, 25, 26, 27, 28, 29, 30, 31, 32, 35, 40, 45, 50, 55, 60, 65, 70, 75, 80, 85, 90, 95, or 100, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range.

Any suitable biological molecule containing at least one tryptophan can be utilized with embodiments described herein. Illustrative, but non-limiting examples of biological molecules (for example, polypeptides) that can be used include octreotide acetate (about 1 kDa), leuprolide (about 1.2 kDa), exenatide (about 4.2 kDa), thioredoxin (about 11.6 kDa), β₂-microglobulin (about 11.7 kDa), lysozyme (14.3 kDa), chymotrypsinogen (about 25 kDa), and bovine carbonic anhydrase II (about 29 kDa), among other suitable biological molecules.

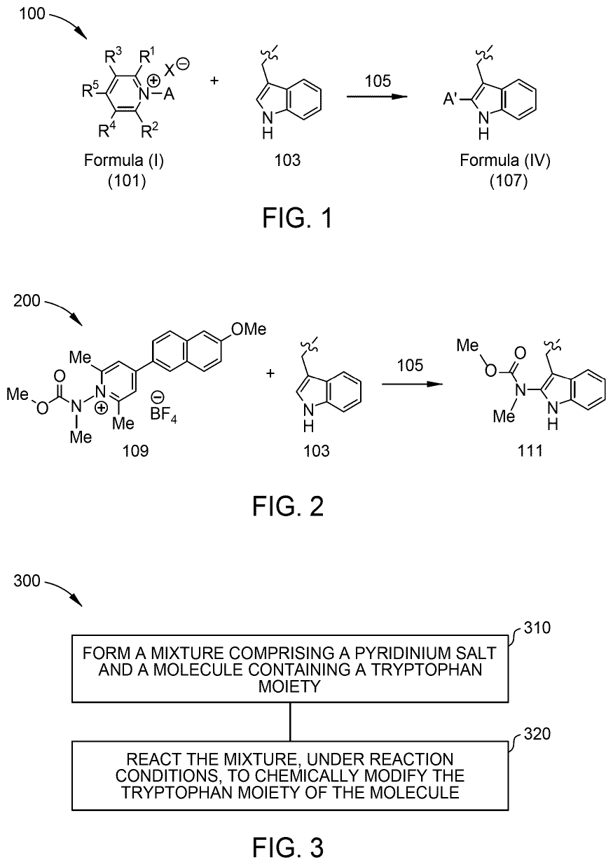

In some embodiments, methods described herein can be used to covalently modify tryptophan. FIG. 1 shows a non-limiting reaction diagram 100 for covalently modifying tryptophan according to at least one embodiment of the present disclosure. In FIG. 1, compound 101 is the pyridinium salt of formula (I) and 103 is a tryptophan residue/moiety, where the wavy bond represents a connection to, for example, a biological molecule (e.g., polypeptide, protein, or proteome). Reacting a mixture comprising a pyridinium salt (compound 101) with a tryptophan residue 103 of a biological molecule, under reaction conditions 105, forms a biological molecule containing a modified tryptophan 107 of formula (VI). The modified tryptophan is represented by formula (VI):

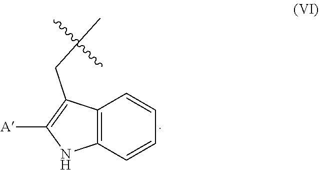

In formula (VI), the wavy bond represents a connection to the biological molecule (e.g., polypeptide, protein, or proteome), biologically-derived molecule, or synthetic molecule. In some embodiments, A′ of formula (VI) is A of formula (I). The reaction is a covalent modification (or chemical modification) of the carbon that is alpha to the nitrogen of the indole ring. After the modification, the biological molecule containing the modified tryptophan (for example, the polypeptide containing a modified tryptophan 107) can be called a conjugate.

An illustrative, but non-limiting, reaction diagram 200 of a tryptophan modification is shown in FIG. 2. In the non-limiting example shown in FIG. 2, reaction of a mixture comprising pyridinium salt 109 and tryptophan residue 103, under reaction conditions 105, forms a carbamate labeled tryptophan residue 111.

FIG. 3 shows selected operations of a method 300 of modifying tryptophan according to at least one embodiment of the present disclosure. As described above, the tryptophan can be present, for example, by itself (a single amino acid), or as a portion of a compound, or as a portion of a biological molecule (for example, a polypeptide, a protein, or a proteome). More than one tryptophan can be present in the compound or biological molecule. For clarity, the description may be illustrated with reference to a polypeptide, though embodiments apply to tryptophan present in any suitable form (for example, as a single amino acid, a suitable biological molecule, or a suitable biologically derived molecule).

Method 300 begins with forming a mixture comprising a biological molecule containing a tryptophan unit (for example, a polypeptide bearing a tryptophan moiety or residue 103) and a composition comprising a pyridinium salt (for example, a pyridinium salt of formula (I)), ion thereof, or combinations thereof at operation 310. More than one pyridinium salt can be utilized for operation 310 if desired.

The pyridinium salt used for the reaction can exist as a mixture of rotamers or a single rotamer. The mixture of rotamers may have a molar ratio of about 1:1 to about 1.5:1, such as from about 1.1:1 to about 1.4:1, such as from about 1.2:1 to about 1.3:1. In at least one embodiment, a molar ratio of the rotamers can be 1:1, 1.1:1, 1.2:1, 1.3:1, 1.4:1, or 1.5:1, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range.

The mixture comprising the pyridinium salt and the biological molecule containing the tryptophan can include one or more additional components. The one or more additional components can include an antioxidant, an additive, a solvent, or combinations thereof.

The antioxidant can be any suitable antioxidant, such as those produced by cells. Illustrative, but non-limiting, examples of antioxidants include glutathione, ubiquinol, uric acid, bilirubin, coenzyme Q10, lipoic acid, a flavonoid, a phenol, a polyphenol, a phytoestrogen, or combinations thereof, among others.

Suitable solvents can include aqueous solvents such as water, saline, a biologically appropriate liquid, combinations thereof, among others. The solvent can be buffered.

Suitable additives that can be used include compounds useful for buffering the solvent to have a pH value from about 6 to about 8, such as from about 6.5 to about 7.5, such as from about 6.8 to about 7.2. Any of the foregoing numbers can be used singly to describe an open-ended range or in combination to describe a close-ended range. Suitable additives that can be used to make the buffer can include ammonium acetate (NH₄OAc), disodium phosphate (Na₂HPO₄), monopotassium phosphate (KH₂PO₄), tris hydrochloride (Tris-HCl), 2-(N-morpholino)ethanesulfonic acid hydrate (MES), 2,2-bis(hydroxymethyl)-2,2′,2″-nitrotriethanol (Bis-Tris), N-(2-acetamido)iminodiacetic acid (ADA), N-(2-acetamido)-2-aminoethanesulfonic acid (ACES), 1,4-piperazinediethanesulfonic acid (PIPES), ß-hydroxy-4-morpholinepropanesulfonic acid (MOPSO), 1,3-bis[tris(hydroxymethyl)methylamino]propane (Bis-Tris propane), N,N-bis(2-hydroxyethyl)-2-aminoethanesulfonic acid (BES), 3-(N-morpholino)propanesulfonic acid (MOPS), 2-[(2-hydroxy-1,1-bis(hydroxymethyl)ethyl)amino]ethanesulfonic acid (TES), 4-(2-Hydroxyethyl)piperazine-1-ethanesulfonic acid (HEPES), 3-(bis(2-hydroxyethyl)amino)-2-hydroxypropane-1-sulfonic acid (DIPSO), 4-(N-Morpholino)butanesulfonic acid (MOBS), 2-hydroxy-3-[tris(hydroxymethyl)methylamino]-1-propanesulfonic acid (TAPSO), 2-amino-2-(hydroxymethyl)-1,3-propanediol (Tris or Trizma™), 2-hydroxy-3-(4-(2-hydroxyethyl)piperazin-1-yl)propane-1-sulfonic acid (HEPPSO), piperazine-1,4-bis(2-hydroxypropanesulfonic acid) dihydrate (POPSO), 4-(2-hydroxyethyl)-1-piperazinepropanesulfonic acid (EPPS), N-[tris(hydroxymethyl)methyl]glycine (Tricine), diglycine (Gly-Gly), N,N-bis(2-hydroxyethyl)glycine (Bicine), N-(2-hydroxyethyl)piperazine-N′-(4-butanesulfonic acid) (HEPBS), N-[tris(hydroxymethyl)methyl]-3-aminopropanesulfonic acid (TAPS), among others.

Additional additives that can be used include detergents (such as Tween-20), a chelating agent (such as ethylenediaminetetraacetic acid (EDTA)), and/or salts such as sodium chloride (NaCl), potassium chloride (KCl), magnesium chloride (MgCl₂), among others.

Operation 310 can optionally include utilizing a non-reactive gas, such as nitrogen, argon, or combinations thereof. For example, a mixture comprising the biological molecule and the pyridinium salt can be used with these or other non-reactive gases to degas various components or otherwise remove oxygen from the mixture. Operation 310 can also include stirring, mixing, agitation, or combinations thereof. In some embodiments, the mixture can be stirred, mixed, or agitated for any suitable period, such as about 10 min or more, such as from about 30 min to about 5 h, such as from about 1 h to about 4 h, such as from about 2 h to about 3 h, though other periods are contemplated. Any of the foregoing numbers can be used singly to describe an open-ended range or in combination to describe a close-ended range.

As described above, an aqueous buffer can be utilized with the biological molecule and the pyridinium salt. The aqueous buffer can have a pH value of about 6 or more or about 8 or less, such as from about 6.5 to about 7.5, such as from about 6.8 to about 7.2, such as about 6.9, 7, or 7.1. In some embodiments, a pH value of the aqueous buffer can be 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, or 8, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range. In some examples, an aqueous ammonium acetate buffer with a pH of about 6.9 is utilized.

The molar ratio of the various components in the mixture can vary depending on, for example, the level of conversion desired, the number of tryptophan moieties present in the molecule to be modified, among other factors.

In some embodiments, a molar ratio of pyridinium salt to biological molecule in the mixture can be from about 50:1 to about 1:5, such as from about 30:1 to about 1:3, such as from about 25:1 to about 1:2, such as from about 20:1 to about 1:1, such as from about 15:1 to about 2:1, such as from about 10:1 to about 3:1. In at least one embodiment, a molar ratio of pyridinium salt to biological molecule in the mixture can be from about 20:1 to about 3.75:1. In some embodiments, a molar ratio of pyridinium salt to biological molecule in the mixture can be 50:1, 45:1, 40:1, 35:1, 30:1, 25:1, 20:1, 15:1, 12:1, 10:1, 9:1, 8:1, 7:1, 6:1, 5:1, 4:1, 3.75:1, 3:1, 2:1, 1:1, 1:2, 1:3, 1:4, or 1:5, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range.

A molar ratio of antioxidant to pyridinium salt can be any suitable molar ratio, such as from about 10:1 to about 1:5, such as from about 5:1 to about 1:1, such as from about 4:1 to about 1.5:1, such as from about 3:1 to about 2:1. In at least one embodiment, a molar ratio of antioxidant to pyridinium salt can be 10:1, 9:1, 8:1, 7:1, 6:1, 5:1, 4:1, 3:1, 2.5:1, 2:1, 1.5:1, 1:1, 1:2, 1:3, 1:4, or 1:5, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range.

A molar ratio of pyridinium salt to additive used to make the buffer (e.g., ammonium acetate) can be any suitable molar ratio, such as from about 1:300 to about 1:1, such as from about 1:250 to about 5:1, such as from about 1:200 to about 1:10, such as from about 1:150 to about 1:25, such as from about 1:100 to about 1:50. In at least one embodiment, a molar ratio of pyridinium salt to additive used to make the buffer can be from about 1:200 to about 1:13. In some embodiments, a molar ratio of pyridinium salt to additive used to make the buffer can be from about 1:300, 1:275, 1:250, 1:225, 1:200, 1:175, 1:150, 1:125, 1:100, 1:75, 1:50, 1:25, 1:13, 1:10, 1:5, 1:1, or 5:1, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range.

Prior to irradiation with light in operation 320 (discussed below), the mixture can be incubated during operation 310. For example, cell cultures can be incubated with pyridinium salts at about 30° C. to about 40° C. for about 20 minutes to about 4 hours prior to operation 320.

Here, live HEK293T cell cultures were incubated with 1-100 μM concentrations of pyridinium salt 2d for about 60 min at about 37° C. followed by photoirradiation with about 440 nm light for about 20 min at about 4° C.

The mixture comprising the pyridinium salt and the biological molecule (and optionally one or more additional components, such as antioxidant, solvent, etc.) can then be reacted, under reaction conditions (for example, reaction conditions 105), to chemically and/or covalently modify the tryptophan moiety of the biological molecule (for example, the polypeptide containing a modified tryptophan 107) at operation 320. The chemically/covalently-modified tryptophan moiety formed from method 300 represents, for example, a tryptophan-labeled molecule, a tryptophan-tagged molecule, among others.

Operation 320 includes irradiating the mixture with an electromagnetic energy source that emits ultraviolet (UV) and/or visible (VIS) light. The electromagnetic energy source used can be any suitable source. Non-limiting examples of the electromagnetic energy source can include a laser, a light emitting diode (LED), a UV lamp, a plasma-based UV source, a fluorescent lamp, a light bulb, or combinations thereof. The electromagnetic energy source can emit narrow bands or wide bands of electromagnetic energy. The electromagnetic energy source can be combined with an optical filter and/or other optical elements so as to target specific wavelengths or specific ranges of wavelengths. In at least one embodiment, the electromagnetic energy source can be doped to enhance light emission at one or more particular wavelengths.

A wavelength or wavelength range that can be used include for operation 320 include those wavelengths or wavelength ranges of UV, VIS, or combinations thereof. In at least one embodiment, the wavelength or wavelength range utilized can be from about 300 nm to about 700 nm, such as from about 350 nm to about 650 nm, such as from about 400 nm to about 500 nm. In at least one embodiment, the wavelength or wavelength range utilized can be from about 300 nm to about 500 nm, such as from about 300 nm to about 440 nm. In some embodiments, the wavelength or wavelength range (nm) can be 300, 302, 305, 310, 315, 320, 325, 330, 335, 340, 345, 350, 355, 360, 365, 370, 375, 380, 385, 390, 395, 400, 405, 410, 415, 420, 425, 427, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, or 700, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range.

The reaction conditions of operation 320 can include a reaction temperature and a reaction pressure. The reaction temperature of operation 320 can be from about 1° C. or more and/or about 70° C. or less, such as from about 5° C. to about 65° C., such as from about 10° C. to about 50° C., such as from about 15° C. to about 45° C., such as from about 20° C. to about 40° C., such as from about 25° C. to about 35° C. In some embodiments, the reaction temperature (° C.) of operation 320 can be 1, 2, 3, 4, 5, 10, 15, 20, 25, 30, 35, 37, 40, 45, 50, 55, 60, or 65, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range.

The reaction pressure of operation 320 can be any suitable pressure, such as from about 0.7 atm to about 1.3 atm, such as from about 0.8 atm to about 1.2 atm, such as from about 0.9 atm to about 1.1 atm, such as from about 0.95 atm to about 1.05 atm. In some embodiments, the reaction pressure (atm) of operation 320 can be 0.7, 0.75, 0.8, 0.85, 0.9, 0.95, 1, 1.05, 1.1, 1.15, 1.2, 1.25, or 1.3, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range.

The reaction conditions of operation 320 can include stirring, mixing, agitating, or combinations thereof by using suitable devices such as a mechanical stirrer (for example, an overhead stirrer), magnetic stirrer (for example, placing a magnetic stir bar in the vessel above a magnetic stirrer), or other suitable devices.

The reaction conditions of operation 320 can also include a reaction time. The reaction time for operation 320 can be any suitable period, such as about 1 min or more, such as from about 1 min to about 10 h, such as from about 5 min to about 5 h, such as from about 10 min to about 4 h, such as from about 20 min to about 3 h, such as from about 30 min to about 2 h. In some embodiments, the reaction time (minutes) of operation 320 can be 1, 5, 10, 20, 30, 60, 90, 120, 150, 180, 210, 240, 270, 300, 330, 360, 390, 420, 450, 480, 510, 540, 570, or 600, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range.

Operation 320 can optionally include utilizing a non-reactive gas, such as nitrogen, argon, or combinations thereof before, during, and/or after irradiation with electromagnetic energy (for example, light). For example, a mixture comprising the biological molecule and the pyridinium salt can be used with these or other non-reactive gases to degas various components or otherwise remove oxygen from the mixture while irradiated with light.

In some embodiments, operations 310 and 320 can be combined into a single operation. For example, and in some embodiments, a method of modifying tryptophan can include reacting a mixture comprising a pyridinium salt and a biological molecule (and optionally one or more additional components), under reaction conditions, to form a biological molecule containing a modified tryptophan.

In some embodiments, the biological molecule can include one or more tryptophan moieties/residues. For example, the biological molecule can have one, two, three, four, or more tryptophan moieties/residues. When the biological molecule has more than one tryptophan moiety or residue, the resulting product from method 300 can have more than one modified tryptophan in suitable ratios. That is, the resulting product can be mono-labeled, di-labeled, tri-labeled, tetra-labeled, etc., where “labeled” refers to the one or more modified tryptophans of the biological molecule. The amount of labeling can be controlled by, for example, the molar ratio of pyridinium salt to biological molecule, among other factors.

In some embodiments, the biological molecule can include an amino acid moiety or residue besides tryptophan that is reactive. In such embodiments, the methods described herein are selective for tryptophan such that a ratio of the modified tryptophan to the other amino acid that is modified can be from about 2:1 or more, such as about 2:1 to about 100:1, such as from about 5:1 to about 90:1, such as from about 10:1 to about 75:1, such as from about 20:1 to about 50:1. In some embodiments, a ratio of the modified tryptophan to the other amino acid that is modified can be 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, 60:1, 70:1, 80:1, 90:1, or 100:1, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range. The selectivity of modifying the tryptophan over the other amino acid that is not tryptophan can be controlled by, for example, the molar ratio of pyridinium salt to biological molecule, among other factors.

In some embodiments, a biological molecule containing the modified tryptophan can be formed by the following non-limiting procedure. To a flask or vial (or other vessel) is added the desired biological molecule and pyridinium salt. If desired, an antioxidant (such as glutathione) and an additive (such as ammonium acetate) can be added. The resulting mixture can be diluted with an amount of water and then sparged with nitrogen for about 30 minutes prior to irradiation. The actively sparging solution can then be placed in a reactor equipped with an LED lamp and then irradiated for a desired amount of time while maintaining active sparging.

As described herein, the pyridinium salts can be used to transfer useful functional groups to biological molecules such as affinity tags, purification tags, or click handles. As an example of the purification tag, a biotinylated-functionalized pyridinium salt can be utilized to transfer the biotin functional group to a tryptophan moiety. As an example of the click handle, an azide-functionalized pyridinium salt can be utilized to transfer the azide functional group to a tryptophan moiety.

### Methods of Making

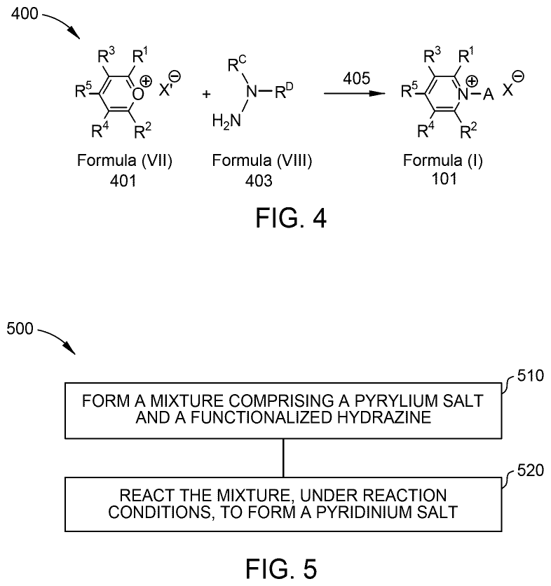

Embodiments of the present disclosure also relate to methods of making pyridinium salts. FIG. 4 shows a non-limiting reaction diagram 400 for forming a pyridinium salt according to at least one embodiment of the present disclosure. In FIG. 4, compound 401 is the pyrylium salt of formula (VII) and compound 403 is a functionalized hydrazine. Reacting a mixture comprising the pyrylium salt (compound 401) and functionalized hydrazine (compound 403), under reaction conditions 405, forms a pyridinium salt of formula (I) (compound 101).

FIG. 5 shows selected operations of a method 500 of making a pyridinium salt of formula (I) according to at least one embodiment of the present disclosure. The method 500 begins with forming a mixture comprising a pyrylium salt (for example, a pyrylium salt of formula (VII)) and a functionalized hydrazine (for example, a functionalized hydrazine of formula (VIII)) at operation 510.

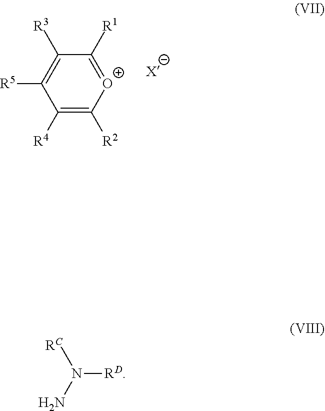

In formula (VII), each of R¹, R², R³, R⁴, and R⁵ is independently hydrogen, unsubstituted hydrocarbyl (such as C1 to C40 unsubstituted hydrocarbyl), substituted hydrocarbyl (such as C1 to C40 substituted hydrocarbyl), or a functional group comprising at least one element of Group 13-17 of the periodic table of the elements, such as those such as those groups described above with respect to R¹, R², R³, R⁴, or R⁵ of formula (I). X′⁻ of formula (VII) is a monoatomic or polyatomic anion such as those described above with respect to X⁻ of formula (I). Illustrative, but non-limiting, examples of a monoatomic anion X′⁻ of formula (VII) can include a halide (F⁻, Cl⁻, Br⁻, and I⁻), an oxide, or combinations thereof. When the anion X⁻ of formula (VII) is a polyatomic anion, X⁻ can be an inorganic anion, an organic anion, or combinations thereof. Illustrative, but non-limiting, examples of a polyatomic anion X⁻ can include tetrafluoroborate (BF₄⁻), trifluoroacetate (TFA, CF₃CO₂⁻), hexafluorophosphate (PF₆⁻), tetrachloroborate (BCl₄⁻), tetrabromoborate (BBr₄⁻), tetraiodoborate (BI₄⁻), nitrate (NO₃⁻), nitrite (NO₂⁻), perchlorate (ClO₄⁻), iodate (IO₃⁻), chlorate (ClO₃⁻), bromate (BrO₃⁻), chlorite (ClO₂⁻), hypochlorite (OCl⁻), hypobromite (OBr⁻) , cyanide (CN⁻), cyanate (OCN⁻), thiocyanate (SCN⁻), permanganate (KMnO₄⁻), hydrogensulfate (HSO₄⁻), hydrogensulfite (HSO₃⁻), sulfonate (R**SO₃⁻), dihydrogen phosphate (H₂PO₄⁻), hydroxide (OH), acetate (CH₃CO₂⁻), formate (HCO₂⁻), hydrogen carbonate (HCO₃⁻), toluenesulfonyl (tosyl, H₃CC₆H₄SO₂⁻), trifluoromethesulfonate (triflate, F₃CSO₃⁻), mesylate (CH₃SO₃⁻), benzoate (BzO, C₇H₅O₂⁻), lactate (C₃H₅O₃⁻), a phosphate ((R**PO₄⁻) such as dibutyl phosphate, (CH₃(CH₂)₃)₂HPO₄⁻)), tetraphenylborate ((C₆H₅)₄B⁻), or combinations thereof. Such anions can be a portion of a larger anion. For example, the sulfonate can have an R** group where the R** is any suitable organic group such as those R groups described herein. Other ions are contemplated.

In some embodiments, X⁻ comprises or is selected from the group consisting of F⁻, Cl⁻, Br⁻, I⁻, BF₄⁻, CF₃CO₂⁻, PF₆⁻, H₃CC₆H₄SO₂⁻, SCN⁻, BCl₄⁻, BBr₄⁻, BI₄⁻, NO₃⁻, F₃CSO₃⁻, CH₃CO₂⁻, CH₃SO₃⁻, C₇H₅O₂⁻, C₃H₅O₃⁻, (CH₃(CH₂)₃)₂HPO₄⁻, (C₆H₅)₄B⁻, and combinations thereof, such as BF₄⁻, CF₃CO₂⁻, and combinations thereof.

N(R^{C})(R^{D}) of the functionalized hydrazine of formula (VIII) becomes the A group of formula (I) while the other nitrogen atom of the functionalized hydrazine becomes the nitrogen atom of the pyridine ring of formula (I).

In formula (VIII), each of R^{C} and R^{D} is, independently, hydrogen, unsubstituted hydrocarbyl, substituted hydrocarbyl, or a functional group comprising at least one element from Group 13-17, such as those groups described above with respect to R¹, R², R³, R⁴, or R⁵ of formula (I). When one or more of R^{C} or R^{D} is a functional group comprising at least one element from Group 13-17, one or more of R^{C} or R^{D} can be, independently, halogen (F, Cl, Br, or I), O, N, Se, Te, P, As, Sb, S, B, Si, Ge, Sn, Pb, and the like, such as NR*₂, C(O)R*, C(C)NR*₂, C(O)OR*, OR*, SeR*, TeR*, PR*₂, AsR*₂, SbR*₂, SR*, SOₓ (where x=2 or 3), BR*₂, SiR*₃, GeR*₃, SnR*₃, PbR*₃, and the like, where R* can be, independently, hydrogen or unsubstituted hydrocarbyl, or where at least one heteroatom has been inserted within the unsubstituted hydrocarbyl.

One or more of R^{C} or R^{D} of formula (VIII) can have, independently, any suitable number of carbon atoms such as from 1 to 40 carbon atoms, such as from 1 to 20 carbon atoms, such as from 1 to 12 carbon atoms, such as from 1 to 10 carbon atoms, such as from 1 to 8 carbon atoms, such as from 1 to 5 carbon atoms, 1 to 4 carbon atoms, or from 3 to 8 carbon atoms. In some embodiments, the number of carbon atoms can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or ranges thereof, though other numbers of carbon atoms are contemplated. Each of the foregoing numbers can be preceded by the word “about,” “at least about,” “less than about,” or “more than about,” and any of the foregoing numbers can be used singly to describe an open-ended range or in combination to describe a close-ended range. One or more of R^{C} or R^{D} can be, independently, linear or branched, saturated or unsaturated, cyclic or acyclic, monocyclic or polycyclic, aromatic or not aromatic. Regarding saturation, one or more of R^{C} or R^{D} of formula (VIII) can be, independently, fully saturated, partially unsaturated, or fully unsaturated.

In at least one embodiment, one or more of R^{C} or R^{D} of formula (VIII) can be, independently, an alkyl group having from 1 to 40 carbon atoms (such as from 1 to 20 carbon atoms) such as methyl, ethyl, n-propyl, isopropyl, n-butyl, iso-butyl, sec-butyl, and tert-butyl, pentyl, hexyl, heptyl, octyl, ethyl-2-hexyl, isooctyl, nonyl, n-decyl, isodecyl, or isomers thereof; a cycloaliphatic group having from 3 to 40 carbon atoms, such as from 3 to 20 carbon atoms such as, for example, cyclopentyl or cyclohexyl; an aryl group having from 5 to 40 carbon atoms, such as from 6 to 20 carbon atoms such as, for example, phenyl, naphthyl, fluorenyl; or any combination thereof. Any of the foregoing numbers can be used singly to describe an open-ended range or in combination to describe a close-ended range.

In some embodiments, one or more of R^{C} or R^{D} of formula (VIII) can be, independently, an unsubstituted aryl (such as such as unsubstituted phenyl, unsubstituted naphthyl, unsubstituted fluorenyl), a substituted aryl (such as alkylphenyl, alkoxyphenyl, halogenated phenyl, alkoxynaphthyl, alkylnaphthyl, a halogenated naphthyl), an unsubstituted heteroaryl (such as pyrrole, imidazole, pyrazole, thiophene, furan), a substituted heteroaryl, among others. When one or more of R^{C} or R^{D} of formula (VIII) is an unsubstituted aryl, a substituted aryl, an unsubstituted heteroaryl, or a substituted heteroaryl, each of the unsubstituted aryl, the substituted aryl, the unsubstituted heteroaryl, and the substituted heteroaryl can, independently, have from 4 to 40 carbon atoms, such as from 5 to 20 carbon atoms, such as from 6 to 12 carbon atoms, such as from 6 to 10 carbon atoms.

In at least one embodiment, the functionalized hydrazine is represented by formula (VIIIa), formula (VIIIb), or formula (VIIIc):

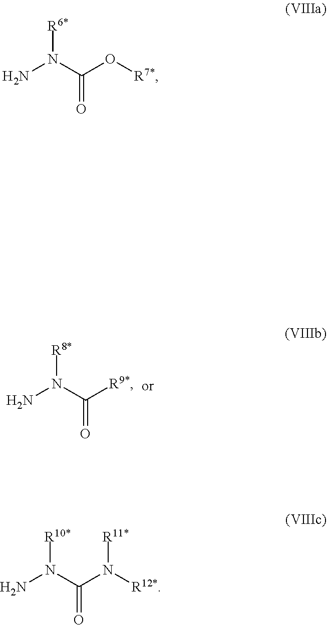

In formulas (VIIIa)-(VIIIc), each of R⁶*, R⁷*, R⁸*, R⁹*, R¹⁰*, R¹¹*, and R¹²* is, independently, hydrogen, unsubstituted hydrocarbyl, substituted hydrocarbyl, or a functional group comprising at least one element from Group 13-17, such as those groups described above for R⁶, R⁷, R⁸, R⁹, R¹⁰, R¹¹, and R¹² with respect to formulas (IIIa)-(IIIc). When one or more of R⁶*, R⁷*, R⁸*, R⁹*, R¹⁰*, R¹¹*, or R¹²* of formulas (VIIIa)-(VIIIc) is a functional group comprising at least one element from Group 13-17, one or more of R⁶*, R⁷*, R⁸*, R⁹*, R¹⁰* R¹¹*, or R¹²* can be, independently, halogen (F, Cl, Br, or I), O, N, Se, Te, P, As, Sb, S, B, Si, Ge, Sn, Pb, and the like, such as NR*₂, C(O)R*, C(C)NR*₂, C(O)OR*, OR*, SeR*, TeR*, PR*₂, AsR*₂, SbR*₂, SR*, SOₓ (where x=2 or 3), BR*₂, SiR*₃, GeR*₃, SnR*₃, PbR*₃, and the like, where R* can be, independently, hydrogen or unsubstituted hydrocarbyl, or where at least one heteroatom has been inserted within the unsubstituted hydrocarbyl.

One or more of R⁶*, R⁷*, R⁸*, R⁹*, R¹⁰*, R¹¹*, or R¹²* of formulas (VIIIa)-(VIIIc) can have, independently, any suitable number of carbon atoms such as from 1 to 40 carbon atoms, such as from 1 to 20 carbon atoms, such as from 1 to 12 carbon atoms, such as from 1 to 10 carbon atoms, such as from 1 to 8 carbon atoms, such as from 1 to 5 carbon atoms, 1 to 4 carbon atoms, or from 3 to 8 carbon atoms. In some embodiments, the number of carbon atoms can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or ranges thereof, though other numbers of carbon atoms are contemplated. Each of the foregoing numbers can be preceded by the word “about,” “at least about,” “less than about,” or “more than about,” and any of the foregoing numbers can be used singly to describe an open-ended range or in combination to describe a close-ended range. One or more of R⁶*, R⁷*, R⁸*, R⁹*, R¹⁰*, R¹¹*, or R¹²* of formulas (VIIIa)-(VIIIc) can be, independently, linear or branched, saturated or unsaturated, cyclic or acyclic, monocyclic or polycyclic, aromatic or not aromatic. Regarding saturation, one or more of R⁶*, R⁷*, R⁸*, R⁹*, R¹⁰*, R¹¹*, or R¹²* of formulas (VIIIa)-(VIIIc) can be, independently, fully saturated, partially unsaturated, or fully unsaturated.

In at least one embodiment, one or more of R⁶*, R⁷*, R⁸*, R⁹*, R¹⁰*, R¹¹*, or R¹²* of formulas (VIIIa)-(VIIIc) can be, independently, an alkyl group having from 1 to 40 carbon atoms (such as from 1 to 20 carbon atoms) such as methyl, ethyl, n-propyl, isopropyl, n-butyl, iso-butyl, sec-butyl, and tert-butyl, pentyl, hexyl, heptyl, octyl, ethyl-2-hexyl, isooctyl, nonyl, n-decyl, isodecyl, or isomers thereof; a cycloaliphatic group having from 3 to 40 carbon atoms, such as from 3 to 20 carbon atoms such as, for example, cyclopentyl or cyclohexyl; an aryl group having from 5 to 40 carbon atoms, such as from 6 to 20 carbon atoms such as, for example, phenyl, naphthyl, fluorenyl; or any combination thereof. Any of the foregoing numbers can be used singly to describe an open-ended range or in combination to describe a close-ended range.

In some embodiments, one or more of R⁶*, R⁷*, R⁸*, R⁹*, R¹⁰*, R¹¹*, or R¹²* of formulas (VIIIa)-(VIIIc) can be, independently, an unsubstituted aryl (such as such as unsubstituted phenyl, unsubstituted naphthyl, unsubstituted fluorenyl), a substituted aryl (such as alkylphenyl, alkoxyphenyl, halogenated phenyl, alkoxynaphthyl, alkylnaphthyl, a halogenated naphthyl), an unsubstituted heteroaryl (such as pyrrole, imidazole, pyrazole, thiophene, furan), a substituted heteroaryl, among others. When one or more of R⁶*, R⁷*, R⁸*, R⁹*, R¹⁰*, R¹¹*, or R¹²* of formulas (VIIIa)-(VIIIc) is an unsubstituted aryl, a substituted aryl, an unsubstituted heteroaryl, or a substituted heteroaryl, each of the unsubstituted aryl, the substituted aryl, the unsubstituted heteroaryl, and the substituted heteroaryl can, independently, have from 4 to 40 carbon atoms, such as from 5 to 20 carbon atoms, such as from 6 to 12 carbon atoms, such as from 6 to 10 carbon atoms.

The mixture comprising the comprising the pyrylium salt and the functionalized hydrazine can further optionally include one or more additional components such as a solvent. Non-limiting examples of solvents can include hydrocarbons such as n-pentane, n-hexane, n-heptane, cyclopentane, and cyclohexane; aromatic hydrocarbons such as benzene, toluene, and xylene; alcohols such as methanol, ethanol, 1-propanol, isopropanol, 1-butanol, isobutanol, n-butanol, t-butanol, 1-pentanol, and ethylene glycol; ketones such as acetone, methyl ethyl ketone, and methyl isobutyl ketone; ethers such as diethyl ether, methyl tert-butyl ether (MTBE), petroleum ether, and tetrahydrofuran; chlorinated solvents such as dichloromethane, chloroform, carbon tetrachloride, ethylene dichloride, and chlorobenzene; esters such as ethyl acetate, mixed heptyl acetate esters, mixed hexyl acetate esters, and mixed octyl acetate esters. Other non-limiting examples of solvents can include acetonitrile, dimethyl formamide, dimethyl acetamide, and dimethyl sulfoxide, among others. A single solvent or a mixture of two or more solvents can be used.

Operation 510 can optionally include an injection time. The injection time is the period over which the functionalized hydrazine of formula (VIII) is added to the pyrylium salt of formula (VII). The functionalized hydrazine can be added to the pyrylium salt as a solution in a solvent. For example, the functionalized hydrazine can be diluted in one or more solvents prior to adding to the pyrylium salt. The pyrylium salt can also be diluted in one or more solvents prior to adding the functionalized hydrazine. The injection time of operation 510 can be from about 1 minutes to about 5 hours, such as from about 5 minutes to about 3 hours, such as from about 10 minutes to about 1.5 hours, such as from about 20 minutes to about 1 hour, though other periods are contemplated. Any of the foregoing numbers can be used singly to describe an open-ended range or in combination to describe a close-ended range.

Operation 510 can optionally include utilizing a non-reactive gas, such as nitrogen, argon, or combinations thereof. For example, a mixture comprising the pyrylium salt and the functionalized hydrazine can be used with these or other non-reactive gases to degas various components or otherwise remove oxygen from the mixture. Operation 510 can also include stirring, mixing, agitation, or combinations thereof. In some embodiments, the mixture can be stirred, mixed, or agitated for any suitable period, such as about 10 min or more, such as from about 30 min to about 5 h, such as from about 1 h to about 4 h, such as from about 2 h to about 3 h, though other periods are contemplated. Any of the foregoing numbers can be used singly to describe an open-ended range or in combination to describe a close-ended range.

In some embodiments, a molar ratio of the functionalized hydrazine to the pyrylium salt can be from about 1:1 to about 5:1, such as from about 1.1:1 to about 3:1, such as from about 1.3:1 to about 2.5:1, such as from about 1.5:1 to about 2:1. In at least one embodiment, a molar ratio of the functionalized hydrazine to the pyrylium salt can be 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, or 5:1, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range.

Method 500 further includes reacting the mixture comprising the pyrylium salt and the functionalized hydrazine (and optionally one or more additional components, such as a solvent) can then be reacted, under reaction conditions to form the pyridinium salt of formula (I) at operation 520. The pyridinium salt formed may exist as rotamers. The mixture of rotamers can be used together in, for example, reacting with a tryptophan residue/moiety. If desired, the rotamers can be separated prior to use. The mixture of rotamers may have a molar ratio of about 1:1 to about 1.5:1, such as from about 1.1:1 to about 1.4:1, such as from about 1.2:1 to about 1.3:1. In at least one embodiment, a molar ratio of the rotamers can be 1:1, 1.1:1, 1.2:1, 1.3:1, 1.4:1, or 1.5:1, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range.

Reaction conditions of operation 520 can include a reaction temperature and a reaction pressure. The reaction temperature of operation 520 can be any suitable reaction temperature such as from about 20° C. to about 130° C., such as from about 30° C. to about 120° C., such as from about 40° C. to about 110° C., such as from about 50° C. to about 100° C., such as from about 60° C. to about 90° C., such as from about 70° C. to about 80° C. In some embodiments, the reaction temperature (° C.) of operation 520 can be 20, 25, 30, 35, 40, 45, 50, 55, 60, 65, 70, 75, 80, 85, 90, 95, 100, 105, 110, 115, 120, 125, or 130, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range.

The reaction pressure of operation 520 can be any suitable pressure such as from about 0.7 atm to about 1.3 atm, such as from about 0.8 atm to about 1.2 atm, such as from about 0.9 atm to about 1.1 atm, such as from about 0.95 atm to about 1.05 atm. In some embodiments, the reaction pressure (atm) of operation 520 can be 0.7, 0.75, 0.8, 0.85, 0.9, 0.95, 1, 1.05, 1.1, 1.15, 1.2, 1.25, or 1.3, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range.

The reaction conditions of operation 520 can also include a reaction time. The reaction time for operation 520 can be any suitable period, such as about 30 min or more, such as from about 30 min to about 48 h, such as from about 2 h to about 36 h, such as from about 3 h to about 24 h, such as from about 5 h to about 18 h, such as from about 8 h to about 12 h. In some embodiments, the reaction time (hours, h) of operation 520 can be 0.2, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48, or ranges thereof, though other values are contemplated. Each of the foregoing numbers can be preceded by the word “about,” “at least about,” “less than about,” or “more than about,” and any of the foregoing numbers can be used singly to describe an open-ended range or in combination to describe a close-ended range.

The reaction conditions of operation 520 can include stirring, mixing, agitating, or combinations thereof by using suitable devices such as a mechanical stirrer (for example, an overhead stirrer), magnetic stirrer (for example, placing a magnetic stir bar in the vessel above a magnetic stirrer), or other suitable devices. The reaction conditions of operation 520 can optionally include utilizing a non-reactive gas, such as nitrogen, argon, or combinations thereof. For example, a non-reactive gas can be used to degas various components or otherwise remove oxygen from the mixture during operation 520.

If desired, the method 500 can optionally include an anion exchange where the anion is exchanged from, for example, a tetrafluoroborate to a trifluoroacetate, among other anion exchanges. The anion exchange can be performed by suitable methods. For example, an anion exchange resin (for example, an Amberlyst resin, such as Amberlyst A-26 (OH⁻) form) can be used. For anion exchange, the anion exchange resin is converted to the X⁻ form, where X⁻ is the ion that will replace the original anion of the pyridinium salt.

Here, and as a non-limiting example, a solution of the pyridinium salt in a selected solvent can be passed through an anion exchange resin (X⁻ form), and optionally washed with the selected solvent. Any suitable solvent can be used for anion exchange such as alcohols (for example, methanol or ethanol), chlorinated solvents (such as dichloromethane, chloroform, or carbon tetrachloride), acetonitrile, or water. Mixtures of solvents in suitable proportions can be utilized for the anion exchange.

In some embodiments, at least a portion of operations 510 and 520 can be combined into a single operation. For example, and in some embodiments, a method of forming a pyridinium salt can include reacting and/or irradiating a mixture comprising a pyrylium salt and a functionalized hydrazine (and optionally one or more additional components), under reaction conditions, to form the pyridinium salt.

In some embodiments, a pyridinium salt can be formed by the following non-limiting procedure. To a flask or vial (or other vessel) is added a pyrylium salt and ethanol, and then evacuated and refilled with N₂. The mixture can be stirred and a functionalized hydrazine can be added dropwise (such as about 20 minutes), and the resulting solution can be stirred at a selected temperature (such as about 60° C.) for a desired amount of time (such as about 3 to about 24 hours).

Embodiments of the present disclosure generally relate to pyridinium salts, compositions thereof, and to methods of use. Overall, pyridinium salts described herein can enable covalent tryptophan modification in molecules such as polypeptides and proteins using light such as visible light and/or UV light. The transferring group (the A group of formula (I)) can be used to install functional handles useful for, e.g., chemoproteomics and general bioconjugation strategies on to polypeptides. In addition, pyridinium salts described herein are useful for chemoproteomic profiling, enabling enrichment of the tryptophan-ome from both lysates and live cell culture.

The following examples are put forth so as to provide those of ordinary skill in the art with a complete disclosure and description of how to make and use embodiments of the present disclosure, and are not intended to limit the scope of embodiments of the present disclosure. Efforts have been made to ensure accuracy with respect to numbers used but some experimental errors and deviations should be accounted for.

### Examples

The following non-limiting examples illustrate, for example, a method for covalent tryptophan modification. Although N-carbamoyl pyridinium salts are described in the examples, other pyridinium salts (as described herein) can be utilized. The covalent modification generally uses light. The pyridinium salts (probes) were assayed against eight non-limiting peptides and proteins. In some non-limiting examples, it was found that micromolar (μM) concentrations of the probe and/or short irradiation times (10-60 min) with light led to efficient reactivity toward tryptophan residues (such as surface-exposed tryptophan residues). As shown in the non-limiting examples, the carbamate transferring group can be used to transfer useful functional groups to proteins including affinity tags and click handles. As further described in the examples, functionalized pyridinium salts (e.g., biotinylated and azide-functionalized pyridinium salts) were used for tryptophan profiling in HEK293T lysates and in situ in HEK293T cells using irradiation. In a non-limiting example, peptide-level enrichment from live cell labeling experiments identified 290 tryptophan modifications, with 82% selectivity for tryptophan modification over other x-amino acids. The results illustrate, for example, that embodiments described herein can be utilized to react with, identify, and/or quantify reactive tryptophan residues of live cells.

### A. Materials and Test Methods

Nuclear magnetic resonance (NMR) spectra were acquired at ambient temperature unless otherwise stated using either a Bruker DRX-400 or an Avance III 600 spectrometer. Chemical shifts (δ) are reported in ppm and coupling constants (J) are reported in Hz. Data are reported in the following format: Chemical shift (multiplicity, coupling constants, number of protons). The following convention is used to report multiplicity: s=singlet, d=doublet, t=triplet, q=quartet, qn=quintet, sext=sextet, sept=septet, m=multiplet, br=broad. ¹⁹F NMR spectra were recorded using fluorobenzene (C₆H₅F; δF=−115.3 ppm in acetonitrile-d3) as an internal standard. Pyridinium salts can exist as rotamers. Where possible, NMR signals are assigned integration values equal to the number of protons associated with the given functional group of the given rotamer. Signals where rotameric peaks are overlapping are assigned hydrogens based directly on their integration values. Infrared (IR) spectra were collected on a Perkin Elmer Spectrum Two FT-IR equipped with an ATR probe. Absorptions are reported in wavenumbers (cm-1). Analytical thin layer chromatography (TLC) was performed using Merck pre-coated glass backed silica gel plates (TLC Silica gel 60 F254). TLC plates were visualized using either UV-light (254 nm), potassium permanganate or ceric ammonium molybdate staining solutions. Flash column chromatography was performed under positive pressure of compressed air using SiliCycle SiliaFlash® P60 silica gel (230-400 mesh) unless otherwise stated. High-resolution mass spectra were obtained using a SCIEX 5800 MALDI TOF-TOF. MS/MS spectra of purified protein modification reactions were obtained using a ThermoFisher Vanquish LTQ XL unless specified otherwise. MS/MS spectra were manually processed using either Mmass or Lorikeet. Mass spectra were deconvolved using ThermoFisher Biopharma software and plotted using Graphpad Prism. Absorption spectra were acquired on a Jasco V-670 spectrophotometer. Emission spectra were acquired on a Cary Eclipse fluorescence spectrophotometer. Lifetime data was obtained with a Horiba EasyLife X lifetime instrument. Protein modification reactions were monitored and evaluated by Liquid Chromatography-Mass Spectrometry (LC/MS) using a Thermo-Fisher Vanquish Flex LC coupled to a Thermo-Fisher LTQ XL ion trap mass spectrometer equipped with a heated electrospray ionization (ESI) probe. Conversions were estimated using total ion count (TIC).

Reactions which did not involve modification of proteins were performed in heat-dried glassware using appropriate Schlenk techniques.

The term “equiv.” refers to equivalent. The term “R.T.” refers to room temperature. The term “kDa” refers to kilodaltons. “Trp” refers to tryptophan.

2,4,6-trimethyl-1-(methyl(((5-((3aS,4S,6aR)-2-oxohexahydro-1H-thieno[3,4-d]imidazol-4-yl)pentyl)oxy)carbonyl)amino)pyridin-1-ium tetrafluoroborate (1a), 2,6-dimethyl-4-phenylpyrylium tetrafluoroborate (S1), and methyl 1-methylhydrazine-1-carboxylate (M1MHC) were synthesized by known methods.

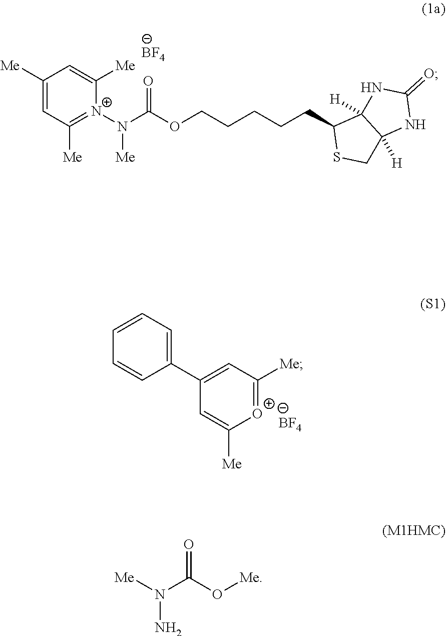

Octreotide acetate was purchased from Apex Bio. Leuprolide acetate was purchased from SelleckChem. Exenatide acetate Bio-X was purchased from Carbosynth. β₂-microglobulin (Human Urine≥98% (SDS-PAGE)) was purchased from Lee Biosolutions. Lysozyme (from chicken egg white, >40,000 units/mg of protein), α-Chymotrypsinogen A from bovine pancreas (essentially salt free, lyophilized powder), Carbonic Anhydrase Isozyme II from bovine erythrocytes (lyophilized powder, >3,000 units/mg), and Thioredoxin from Escherichia coli (recombinant, expressed in E. coli, essentially salt free) were purchased from Sigma Aldrich.

All peptides and proteins were used without additional purification. All other reagents were purchased from commercial sources and used as received.

### A.1. General Liquid Chromatography-Mass Spectrometry (LC/MS) Methods

Method A: solvents: A: 0.1% formic acid in H₂O B: 0.1% formic acid in CH₃CN, method: 20-70% B over 5 min, 70-95% B over 1 min, hold 95% B for 2 min, 95-20% B over 0.5 min, then hold 20% B 1.5 min, column: Aeris™ 3.6 μm WIDEPORE XB-C18 20, LC Column 100×21 mm, m/z range 5-20% B over 0.5 min, then hold 20% B 1.5 min, column: Aeris 3.6 μm WIDEPORE XB-C18 20, LC Column 100×21 mm, m/z range: 500-2000, flow rate: 0.2 mL/min.

Method B: solvents: A: 0.1% formic acid in H₂O B: 0.1% formic acid in CH₃CN, method: 20-70% B over 5 min, 70-90% B over 1 min, 90-20% B over 1 min, then hold 20% B 3 min, column: Aeris™ 3.6 μm WIDEPORE XB-C18 20, LC Column 100×21 mm, m/z range: 800-2000, flow rate: 0.2 mL/min.

Method C: solvents: A: 0.1% formic acid in H₂O B: 0.1% formic acid in CH₃CN, method: 10-50% B over 8 min, 70-90% B over 1 min, hold 90% B for 1 min, 90-10% B over 1 min, then hold 10% B 1 min, column: Kinetex® 1.3 μm C18 100 Å, LC Column 50×2.1 mm, m/z range: 500-2000, flow rate: 0.3 mL/min.

Method D: solvents: A: 0.1% formic acid in H₂O B: 0.1% formic acid in CH₃CN, method: hold 10% B for 0.5 min, 10-40% B over 10.5 min, 40-90% B over 1 min, hold 90% B for 1.5 min, 90-10% B over 0.5 min, then hold 10% B 1 min, column: Kinetex® 1.3 μm C18 100 Å, LC Column 50×2.1 mm, m/z range: 500-2000, flow rate: 0.3 mL/min.

### A.2. General High-Performance Liquid Chromatography (HPLC) Methods:

Method A: solvents: A: 0.1% TFA in H2O, B: 0.1% TFA in CH₃CN, method: hold 5% B 1 min, 5-35% B over 28 min, 35-90% B over 1 min, hold 90% B 2 min, 90-5% B over 1 min, hold 5% B 2 min, UV-Vis: 290, 280, 220 nm, column: Kinetex® 5 μm C18 100 Å, LC Column 150×21.2 mm, flow rate: 10 mL/min.

Method B: solvents: A: 0.1% TFA in H2O, B: 0.1% TFA in CH₃CN, method: hold 10% B 1 min, 10-70% B over 16 min, 70-95% B over 1 min, hold 95% B 3 min, 95-10% B over 1 min, hold 10% B 3 min, UV-Vis: 390, 365, 254 nm, column: Kinetex® 5 μm C18 100 Å, LC Column 150×21.2 mm, flow rate: 10 mL/min.

### B. Small Molecule Synthesis Procedures

### Synthesis of 4-(6-methoxynaphthalen-2-yl)-2,6-dimethylpyrylium tetrafluoroborate (S2)

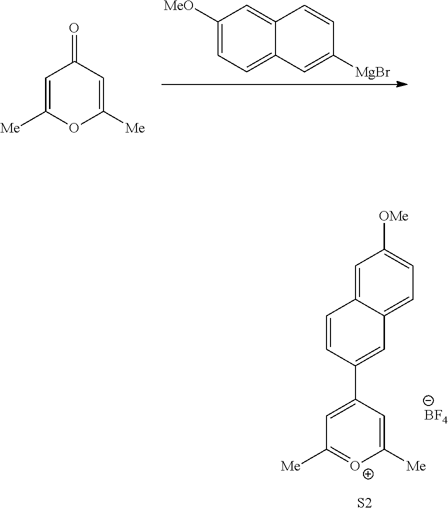

A round-bottom flask equipped with a side arm inlet adapter and stir bar was heat dried under vacuum, allowed to cool to room temperature, and refilled with nitrogen (N₂). The flask was charged with 2,6-dimethyl-gamma-pyrone (0.740 g, 5.96 mmol) and then was evacuated and refilled with N₂. To the flask was added tetrahydrofuran (THF) by syringe (2.9 mL), and the resulting mixture was cooled in an ice batch and stirred. To the stirring, chilled suspension was added 6-methoxy-2-naphthyl magnesium bromide (12.5 mL of a 0.5 M solution in THF, 6.25 mmol) dropwise over 15 minutes. The resulting solution was stirred at 0° C. for 5 minutes and then allowed to warm to room temperature. The solution was stirred vigorously for 5 hours. The mixture was then cooled to 0° C. again before being quenched carefully by dropwise addition of tetrafluoroboric acid (HBF₄, 2.0 mL of 50-55% w/w in ether, 16 mmol). The resulting mixture was allowed to stir for 20 minutes while warming to room temperature. To the mixture was added 10 mL diethyl ether (Et₂O) with stirring, and the resulting mixture was filtered. The filter cake was then triturated in ethanol using sonication, filtered, and washed with ethanol. The resulting solid was finally washed with excess diethyl ether and dried to yield the pyrylium salt (4-(6-methoxynaphthalen-2-yl)-2,6-dimethylpyrylium tetrafluoroborate (S2)) as a reddish-orange solid (1.094 g, 52% yield). ¹H NMR (400 MHZ, CD₃CN) δ: 8.74 (s, 1H), 8.25 (s, 2H), 8.10-8.01 (m, 3H), 7.46-7.41 (m, 1H), 7.39-7.32 (m, 1H), 4.01 (s, 3H), 2.89 (s, 6H). ¹³C NMR (100 MHz, CD₃OD) δ: 177.2, 165.9, 161.9, 139.0, 132.3, 131.8, 128.7, 128.6, 126.3, 124.2, 120.6, 116.7, 105.8, 54.9, 20.0. ¹⁹F NMR (376 MHz, CD₃CN) δ: −152.1—−152.2 (m). Fourier Transform Infrared (FT-IR): 2972.5, 1644.3, 1611.5, 1534.3, 1490.5, 1375.6, 1331.5, 1196.8, 1052.1, 1035.5, 1018.8, 947.0, 874.9, 861.9, 807.8, 518.1, 469.5. High resolution-mass spectrometry (HR-MS) (m/z) Found (M⁺) 265.1019, Calculated C18H1702+265.1223. Melting Point (MP): 201-205° C.

### Synthesis of (3aS,4S,6aR)-4-(5-hydroxypentyl)tetrahydro-1H-thieno[3,4-d)imidazol-2(3H)-one (S3)

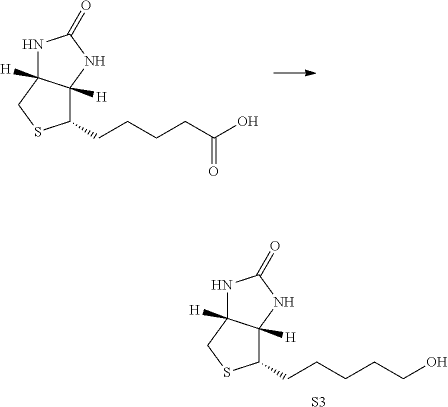

A flame dried, nitrogen backfilled, multi-necked flask fitted with a condenser and a stir bar was charged with lithium aluminum hydride (LiAlH4, 3.50 g, 92.2 mmol) and dry THF (60 mL) was added under nitrogen at room temperature. A suspension of D-biotin (2.42 g, 9.95 mmol) in dry THF (120 mL) was added slowly by syringe (16 gauge needle). Once added the mixture was refluxed for 24 hours then cooled to room temperature then chilled in an ice bath. Water (60 mL) was added by slow addition (0.27 mL/min) by syringe pump at 0° C. An additional 40 mL of water was added dropwise to the chilled mixture followed by 1 M sulfuric acid (H2SO₄, 100 mL). This mixture was stirred for 2 hours at room temperature then extracted with ethyl acetate (5×120 mL). The combined organic phase is then dried over magnesium sulfate (MgSO4), concentrated then adsorbed onto silica (60 mL) using methanol and eluted using 1:9 methanol:ethyl acetate to obtain (3aS,4S,6aR)-4-(5-hydroxypentyl)tetrahydro-1H-thieno[3,4-d)imidazol-2(3H)-one (S3) as a white solid (1.40 g, 60%). ¹H NMR (400 MHZ, CD₃OD) δ: 4.49 (m, 1H), 4.31 (dd, J=8, 4.5 Hz, 1H), 3.56 (t, J=6.6 Hz, 2H), 3.22 (m, 1H), 2.93 (dd, J=12.8, 4.9 Hz, 1H), 2.71 (d, J=12.8, 1H), 1.68-1.81 (m, 1H), 1.35-1.66 (m, 7H). ¹³C NMR (150 MHz, CD₃OD) δ: 164.7, 62.0, 61.5, 60.2, 55.7, 39.6, 32.0, 28.7, 28.4, 25.5. FT-IR: 3333.7, 3216.3, 2928.9, 1674.1, 1463.5, 1072.6. HR-MS (m/z) Found (M+): 231.0979, Calculated C₁₀H₁₉N₂O₂S⁺231.1167. MP: 174-175° C.

### Synthesis of (4R,5S)-4-(6-hydroxyhexyl)-5-methylimidazolidin-2-one (S4)

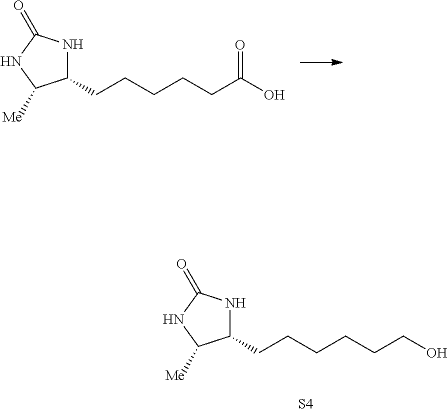 [SMILES: C[C@@H]1NC(=O)N[C@@H]1CCCCCC(=O)O ; C[C@@H]1NC(=O)N[C@@H]1CCCCCCO ; "S4"]

To a flame-dried, nitrogen backfilled, multi-necked flask equipped with a stir bar and reflux condenser was added LiAlH₄ (636 mg, 16.7 mmol). The flask was then evacuated and backfilled with nitrogen followed by the addition of dry THF (10 mL). The mixture was then chilled in an ice bath. A suspension of D-desthiobiotin (400 mg, 1.86 mmol) in dry THF (20 mL) was added dropwise by syringe while chilled. The reaction mixture was brought to room temperature then stirred vigorously while refluxing for 22 hours under N₂. After refluxing the reaction mixture was allowed to cool to room temperature and then chilled in an ice bath. To the stirring suspension was added water (H2O, 20 mL) dropwise by syringe over a 2h period under N₂ by syringe pump. The reaction mixture was then acidified with 1 M hydrochloric acid (HCl, 20 mL). This mixture was extracted with ethyl acetate (6×30 mL) and the combined organic was dried over potassium carbonate (K₂CO₃), filtered then concentrated in vacuo. The crude material was purified by silica gel column chromatography eluting with 10% methanol/dichloromethane (MeOH/DCM) v/v treated with 33% methylamine/ethanol (1 mL per 500 mL eluent). The desired alcohol ((4R,5S)-4-(6-hydroxyhexyl)-5-methylimidazolidin-2-one (S4)) was obtained as a white crystalline solid (279 mg, 75%). ¹H NMR (400 MHZ, CD₃OD) δ: 3.82 (p, J=6.5 Hz, 1H), 3.70 (q, J=6.8 Hz, 1H), 3.5 (t, J=6.6 Hz, 2H), 1.6-1.2 (m, 10H), 1.1 (d, J=6.4 Hz, 3H). ¹³C NMR (150 MHZ, CD₃OD) δ: 164.8, 61.5, 56.1, 51.3, 32.1, 29.4, 29.2, 26, 25.4, 14.2. FT-IR: 3241.9, 2928.0, 1673.3, 1465.9, 1355.4, 1059.7, 727.1, 673.8, 567.4, 522.5. HR-MS (m/z) Found (M⁺): 201.1261, Calculated C₁₀H₂₁N₂O₂⁺201.1603. MP: 91-93° C.

### Synthesis of 5-((3aS,4S,6aR)-2-oxohexahydro-1H-thieno[3,4-d]imidazole-4-yl)pentyl 1-methylhydrazine-1-carboxylate (S5)

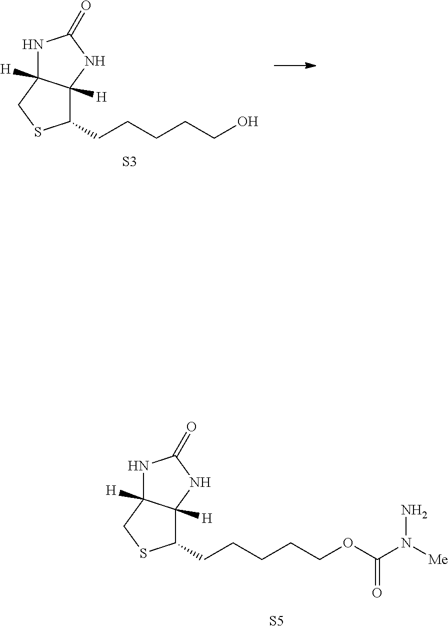

To a flame dried round-bottom flask, backfilled with nitrogen and equipped with a side-arm inlet adaptor and a stir bar is added (3aS,4S,6aR)-4-(5-hydroxypentyl)tetrahydro-1H-thieno[3,4-d)imidazol-2(3H)-one (S3) (777 mg, 3.38 mmol), 1,1-carbonyldiimidazole (1,1-CDI, 827 mg, 5.10 mmol), and dry acetonitrile (CH₃CN, 7 mL) and then stirred vigorously at room temperature for 4 hours. To the reaction mixture was added a second portion of dry CH₃CN (4.5 mL) followed by methylhydrazine (0.760 mL, 13.6 mmol), and the resulting mixture was stirred vigorously for 6 hours. To the stirring mixture was added diethyl ether (10 mL), and the mixture was then sonicated briefly and then filtered. The collected precipitate was dissolved in methanol (50 mL) with mild heating followed by the addition of diethyl ether (150 mL). The resulting solution was then placed in the freezer (−20° C.) for 2 hours to induce precipitation. The resulting precipitate was filtered to yield the desired product (5-((3aS,4S,6aR)-2-oxohexahydro-1H-thieno[3,4-d]imidazole-4-yl)pentyl 1-methylhydrazine-1-carboxylate (S5)) as a white solid (706 mg, 69%). ¹H NMR (400 MHZ, CD₃OD) δ: 4.5 (m , 1H), 4.31 (dd, J=7.9, 4.4 Hz, 1H), 4.1 (t, J=6.4 Hz, 2H), 3.22 (m, J-8.5,5.2,4.8 Hz, 1H), 2.93 (dd, J=12.9,5.1 Hz, 1H), 2.71 (d, J=12.8, 1H), 1.38-1.80 (m, 8H). ¹³C NMR (150 MHZ, CD₃OD) δ: 164.7, 157.9, 65.7, 62.0, 60.2, 55.7, 39.6, 37.2, 28.5, 28.5, 28.3, 25.5. FT-IR: 3224.0, 2939.6, 1699.2, 1682.6, 1464.4, 1186.9, 593. HR-MS (m/z) Found (M+): 303.1345, Calculated C₁₂H₂₃N₄O₃S⁺303.1491. MP: 142-146° C.

### Synthesis of 6-((4R,5S)-5-methyl-2-oxoimidazolidin-4-yl)hexyl 1-methylhydrazine-1-carboxylate (S6)

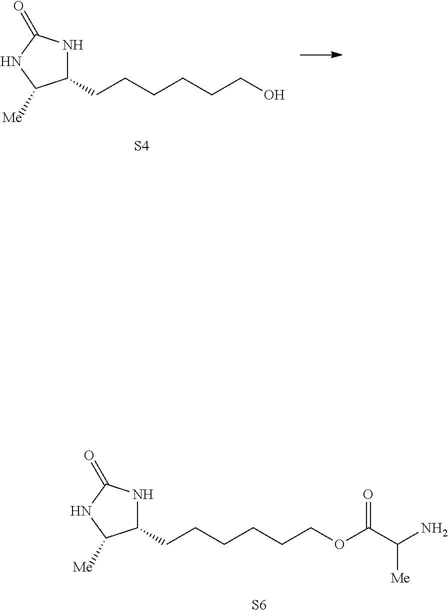 [SMILES: C[C@@H]1NC(=O)N[C@@H]1CCCCCCO ; "S4" ; CC(N)C(=O)OCCCCCC[C@H]1NC(=O)N[C@H]1C ; "S6"]

To a flame dried round-bottom flask, equipped with a stir bar and a side-arm inlet adapter, was added (4R,5S)-4-(6-hydroxyhexyl)-5-methylimidazolidin-2-one (S4) (256 mg, 1.28 mmol) and 1,1-CDI (311 mg, 1.92 mmol). The flask was then then evacuated and refilled with N₂. Dry acetonitrile (4.3 mL) was then added by syringe, and the reaction was stirred for 4 hours at room temperature. To the reaction mixture was then added methylhydrazine (300 μL, 5.11 mmol) was added by syringe and stirred for 16 hours at room temperature. The solvent was then removed using a rotary evaporator. The crude residue was partially purified by silica gel column chromatography using silica gel pH neutralized by adding 33% methylamine/ethanol (1 mL per every 500 mL eluent) then eluting with a gradient of methanol:dichloromethane v/v (1%-10% MeOH). The product obtained from column chromatography was then dissolved in ethyl acetate (50 mL) and washed sequentially with saturated sodium bicarbonate (1×5 mL), water (2×5 mL), and brine (1×5 mL). The organic layer was then dried over K₂CO₃, filtered, and concentrated using a rotary evaporator to yield an inseparable mixture of the desired product (6-((4R,5S)-5-methyl-2-oxoimidazolidin-4-yl)hexyl 1-methylhydrazine-1-carboxylate (S6)) and imidazole as a pale-yellow oil (185 mg, 73% purity by mass). ¹H NMR (400 MHZ, CD₃OD) δ: 4.09 (t, J=7 Hz, 2H), 3.82 (p, J=6.4 Hz, 1H), 3.70 (q, J=7 Hz, 1H), 3.08 (s, 3H), 1.74-1.61 (m, 2H), 1.55-1.27 (m, 8H), 1.11 (d, 3H). ¹³C NMR (150 MHz, CD₃OD) δ: 164.8, 157.9, 65.8, 56.1, 51.3, 37.2, 29.4, 29, 28.6, 25.9, 25.4, 14.2. FT-IR: 3252.9, 2933.7, 2400, 1679.4, 1428.7, 1351.8, 1166.7, 765.7, 523.9. HR-MS (m/z) Found (M+): 273.1633, Calculated C₁₂H₂₅N₄O₃⁺273.1927.

### Synthesis of 6-azido-1-methylhydrazine-1-carboxylate (S8)

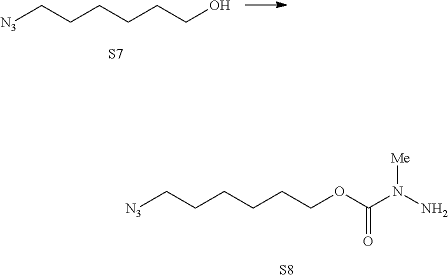

To a flame dried round-bottom flask, equipped with a stir bar and side arm inlet adapter, was added 1,1-CDI (196 mg, 1.21 mmol, 1.3 equiv.). The flask was then evacuated and refilled with N₂. To the flask was added acetonitrile (2.5 mL) and the resulting mixture was then stirred until a homogeneous solution was formed. To the stirring solution was added 6-azidohexan-1-ol (S7) (127 mg, 0.887 mmol, 1.0 equiv.) dropwise by syringe and the reaction was stirred for 3 h at 65° C. The reaction mixture was cooled down to room temperature. To the reaction mixture was then added dropwise methylhydrazine (200 μL, 3.79 mmol, 4.2 equiv.) by syringe and continued stirring for 3h at 65° C. The reaction mixture was cooled down to room temperature and water was added to quench the reaction. The resulting mixture was then extracted with dichloromethane (CH₂Cl₂) (3×5 mL). The combined organic layers were washed sequentially with saturated NaHCO₃(aqueous) (2×5 mL) and H₂O (3×5 mL), dried over MgSO4, filtered, and concentrated using a rotary evaporator to isolate (6-azido-1-methylhydrazine-1-carboxylate (S8)) (147 mg, 96% purity by mass, 77%) as a pale yellow liquid. ¹H NMR (400 MHZ, CD₃OD) δ: 4.19 (s, 2H), 4.05 (t, J=6.54 Hz, 2H), 3.32 (t, J=3.88 Hz, 2H), 3.03 (s, 3H), 1.70-1.56 (m, 4H), 1.46-1.36 (m, 4H). ¹³C NMR (150 MHz, CD₃OD) 8:157.5, 65.3, 55.1, 37.8, 28.7, 28.4, 26.1, 25.2. FT-IR: 3336.6, 2939.5, 2853.4, 2252.9, 2094.6, 1694.3, 1629.7, 1435.9, 1397.9, 1348.9, 1257.4, 1165.6, 1066.0, 988.1, 918.9, 767.1, 732.5, 638.5, 559.6. HR-MS (m/z) Found (M+): 215.1439, Calculated C₈H₁₇N₅O₂⁺215.1382.

### C. General Procedure A: Synthesis of Pyridinium Salts

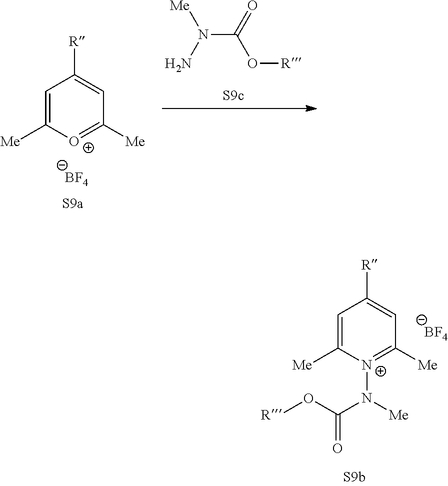

Pyridinium salts S9b were made according to the following general procedure. A round-bottom flask equipped with a stir bar and a side arm inlet adapter was heat dried under vacuum, allowed to cool to room temperature, and refilled with N₂. The flask was charged with the pyrylium salt S9a (1 equiv., 3.6 mmol) and was then evacuated and refilled with N₂. To the flask was added ethanol (EtOH) by syringe, and the resulting mixture stirred vigorously. To the stirring solution was added the functionalized hydrazine S9c (about 1.0-3.0 equiv., optionally in ethanol) dropwise over 20 minutes, and the resulting solution was stirred vigorously at 60° C. for 3-24 hours. The resultant mixture was then concentrated using a rotary evaporator. The resulting residue was purified to provide pyridinium salts S9b. Pyridinium salt purity is validated against an internal standard (1,1,2,2-tetrachloroethane). The resulting pyridinium salts may exist as rotamers.

### Synthesis of 2,6-dimethyl-1-(methyl(((5-((3aS,4S,6aR)-2-oxohexahydro-1H-thieno[3,4-d]imidazole-4-yl)pentyl)oxy)carbonyl)amino)-4-phenylpyridin-1-ium tetrafluoroborate (1b)

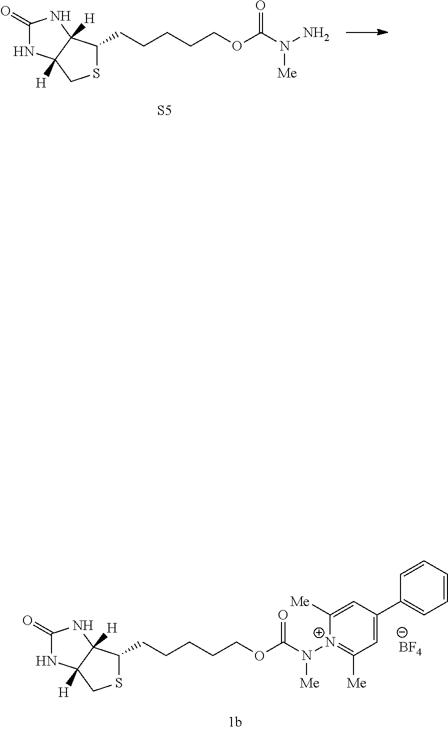

Pyridinium salt 1b was synthesized by adapting general procedure A as follows: To a flame dried round-bottom flask equipped with a stir bar was added 5-((3aS,4S,6aR)-2-oxohexahydro-1H-thieno[3,4-d]imidazole-4-yl)pentyl 1-methylhydrazine-1-carboxylate (S5) (250 mg, 0.826 mmol), 2,6-dimethyl-4-phenylpyrylium tetrafluoroborate (S1) (215 mg, 0.790 mmol), and ethanol (EtOH, 4 mL) then stirred vigorously for 4 hours at room temperature (R.T.) after which diethyl ether (4 mL) was added followed by sonication then decantation of the supernatant. The resulting solid was placed under vacuum then dissolved in 15% isopropanol in dichloromethane (50 mL). This solution was then washed three times with 5 mL portions of a saturated sodium tetrafluoroborate solution, dried over potassium tetrafluoroborate, then concentrated using a rotary evaporator. The resulting solid was then sonicated in ethyl acetate (2 mL), followed by decanting off the supernatant and dried under vacuum. This procedure was repeated twice and yielded the desired pyridinium salt (2,6-dimethyl-1-(methyl(((5-((3aS,4S,6aR)-2-oxohexahydro-1H-thieno[3,4-d]imidazole-4-yl)pentyl)oxy)carbonyl)amino)-4-phenylpyridin-1-ium tetrafluoroborate (1b)) as a pale-yellow powder (120 mg, 26%).

2,6-dimethyl-1-(methyl(((5-((3aS,4S,6aR)-2-oxohexahydro-1H-thieno[3,4-d]imidazole-4-yl)pentyl)oxy)carbonyl)amino)-4-phenylpyridin-1-ium tetrafluoroborate (1b) exists as mixture of rotamers (1.1:1) at 298 K. ¹H NMR (400 MHZ, CD₃OD) δ: 8.37 (s, 2H), 8.34 (s, 2H), 8.06 (m, 2H), 7.67 (m, 3H), 4.49-4.56 (m, 1H), 4.31-4.46 (m, 2H), 4.19-4.29 (m, 2H), 3.57-3.67 (s, 3H), 3.21-3.31 (m, 1H), 3.06-3.13 (m, 0.5H), 2.92-3.01 (m, 1H), 2.83 (s, 6H), 2.74 (m, 1H), 2.62 (s, 0.5H), 1.16-1.91 (m, 11H). ¹³C NMR (150 MHZ, CD₃OD) δ: 159.6, 159.4, 159.4, 154.6, 153.1, 134.9, 134.8, 133.9, 133.8, 131.0, 130.9, 129.6, 129.5, 126, 126, 69.8, 69.5, 63.4, 61.6, 61.5, 57.1, 56.9, 41.0, 40.9, 38, 36.9, 29.9, 29.7, 29.7, 29.6, 29.6, 29.4, 26.8, 19.2, 19.2, 19.1. ¹⁹F NMR (376 MHZ, CD₃CN) δ: −154.6—−154.7 (m). FT-IR: 3403.2, 3234.2, 2927.3, 1694.7, 1629.7, 1445, 1330.6, 1052.4, 1034.2, 761.1. HR-MS (m/z) Found (M-BF₄): 469.2232, Calculated C₂₅H₃₃N₄O₃S⁺469.2273. MP: 110-114° C.

### Synthesis of 1-((methoxycarbonyl)(methyl)amino)-4-(6-methoxynapthalen-2-yl)-2,6-dimethylpyridin-1-ium tetrafluoroborate (2a)

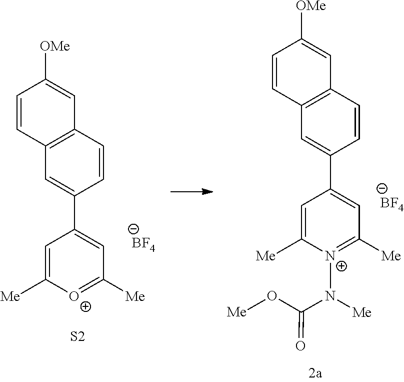

Pyridinium salt 2a was synthesized by adapting general procedure A as follows: To a flame dried round-bottom flask equipped with a stir bar was added pyrylium salt (4-(6-methoxynaphthalen-2-yl)-2,6-dimethylpyrylium tetrafluoroborate (S2)) (0.4937 g, 1.402 mmol), methyl 1-methylhydrazine-1-carboxylate (MIMHC, 0.3245 g, 3.117 mmol), and ethanol (EtOH, 5.0 mL). The reaction was allowed to stir vigorously for 3.5 hours at 60-70° C. The suspension was decanted and the resulting precipitate was then triturated in ethyl acetate, filtered, and dried under vacuum to yield pyridinium salt (1-((methoxycarbonyl)(methyl)amino)-4-(6-methoxynapthalen-2-yl)-2,6-dimethylpyridin-1-ium tetrafluoroborate (2a)) as a yellow powder (0.3411 g, 56% yield).

1-((methoxycarbonyl)(methyl)amino)-4-(6-methoxynapthalen-2-yl)-2,6-dimethylpyridin-1-ium tetrafluoroborate (2a) is a 1.2:1 mixture of rotamers at 300K. ¹H NMR (400 MHZ, CD₃OD) δ: 8.50 (s, 1H), 8.23 (s, 2H), 8.04-7.90 (m, 3H), 7.37 (s, 1H), 7.32-7.25 (m, 1H), 3.95 (s, 3H), 3.91 (s, 1.7H), 3.74 (s, 1.4H), 3.51 (s, 1.6H), 3.48 (s, 1.4H), 2.72 (s, 6H). ¹³C NMR (150 MHz, CD₃OD) δ: 160.2 (2C), 157.8, 157.6, 157.2, 153.7, 152.3, 137.0, 136.9, 131.1 (2C), 128.7, 128.6, 128.5, 128.4 (2C), 127.9 (2C), 124.4, 124.2 (2C), 120.2, 106.0, 55.4, 54.8 (2C), 37.3, 36.5, 18.5, 18.4. ¹⁹F NMR (376 MHz, CD₃CN) δ: −152.1—−152.2 (m). FT-IR: 2967.9, 1732.4, 1611.4, 1563.0, 1493.2, 1456.8, 1327.1, 1266.1, 1196.8, 1048.0, 1018.96, 865.6, 853.5, 519.27, 471.3. HR-MS (m/z) Found (M+): 351.1598, Calculated C₂₁H₂₃N₂O₃⁺351.1703. MP: 206-211° C.

### Synthesis of 4-(6-methoxynaphthalen-2-yl)-2,6-dimethyl-1-(methyl(((5-((3aR,4R,6aS)-2-oxohexahydro-1H-thieno[3,4-d]imidazol-4-yl)pentyl)oxy)carbonyl)amino)pyridin-1-ium tetrafluoroborate (2b)

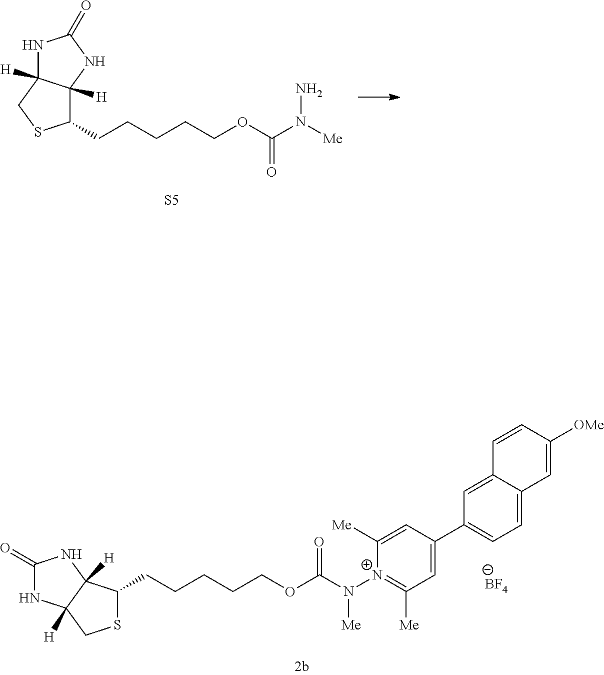

Pyridinium salt 2b was synthesized by adapting general procedure A as follows: To a flame dried round-bottom flask equipped with a stir bar was added 4-(6-methoxynaphthalen-2-yl)-2,6-dimethylpyrylium tetrafluoroborate (S2) (121 mg, 0.340 mmol, 1.0 equiv.) and 5-((3aS,4S,6aR)-2-oxohexahydro-1H-thieno[3,4-d]imidazole-4-yl)pentyl 1-methylhydrazine-1-carboxylate (S5) (134 mg, 0.440 mmol, 1.3 equiv.) in ethanol (5.0 mL). The reaction mixture was allowed to stir vigorously for 3 hours at 65° C. The reaction mixture was then allowed to cool down to room temperature and the solvent was removed using a rotary evaporator. The resulting yellow residue was then dissolved in dichloromethane and washed with saturated sodium tetrafluoroborate (NaBF₄, aqueous) (3×5 mL). The resulting solution was dried over NaBF₄ followed by filtering and concentrating using a rotary evaporator. The resulting yellow solid residues was sonicated and triturated with diethyl ether, filtered and dried under reduced pressure to yield pyridinium salt (4-(6-methoxynaphthalen-2-yl)-2,6-dimethyl-1-(methyl(((5-((3aR,4R,6aS)-2-oxohexahydro-1H-thieno[3,4-d]imidazol-4-yl)pentyl)oxy)carbonyl)amino)pyridin-1-ium tetrafluoroborate (2b)) (145 mg, 72%) as a yellow solid.

4-(6-methoxynaphthalen-2-yl)-2,6-dimethyl-1-(methyl(((5-((3aR,4R,6aS)-2-oxohexahydro-1H-thieno[3,4-d]imidazol-4-yl)pentyl)oxy)carbonyl)amino)pyridin-1-ium tetrafluoroborate (2b) exists as a 1.2:1 mixture of rotamers at 298 K. ¹H NMR (400 MHZ, CD₃OD) δ: 8.55 (d, J=9.26 Hz, 1H), 8.28 (d, J=9.58 Hz, 2H), 8.01 (m, 3H), 7.41 (s, 1H), 7.33 (t, J=4.31 Hz, 1H), 5.09 (s (br), 2H), 4.51-4.40 (m, 0.5H), 4.39-4.24 (m, 1H), 4.21 (t, J=6.17 Hz, 1H), 4.14-4.06 (m 0.5H), 3.99 (s, 3H), 3.54 (s, 3H), 3.26-3.16 (m, 0.5H), 3.05-2.97 (m, 0.5H), 2.97-2.86 (m, 1H), 2.76 (s, 6H), 2.68 (d, J=12.76 Hz, 0.5H), 2.54 (d, J=12.76 Hz, 0.5H), 1.86-1.68 (m, 1H), 1.67-1.45 (m, 4H), 1.42 (t, J=10.72 Hz, 1H), 1.35-1.08 (m, 2H), 0.96-0.83 (m, 1H). ¹³C NMR (150 MHz, CD₃OD) δ: 163.0, 160.3, 160.3, 157.8, 157.6, 157.6, 157.3, 157.2, 153.2, 151.8, 137.0, 137.0, 131.1, 131.0, 129.8, 129.7, 128.6, 128.6, 128.4, 128.0, 127.9, 124.5, 124.3, 124.2, 120.3, 106.1, 68.4, 68.2, 61.5, 61.5, 59.8, 59.7, 55.5, 55.4, 55.3, 40.1, 40.0, 37.3, 36.2, 31.3, 28.5, 28.4, 28.2, 27.9, 25.5, 25.4, 18.6, 18.5 13.4. ¹⁹F NMR (376 MHz, CD₃CN) δ: −152.6—−152.7 (m). FT-IR: 1727.3, 1688.5, 1613.7, 1560.6, 1491.0, 1454.5, 1439.4, 1326.3, 1263.6, 1227.3, 1191.9, 1169.7, 1051.5, 1022.0, 866.7, 854.6, 806.1, 757.6, 678.8, 630.3, 518.2, 469.7. HR-MS (m/z) Found (M-BF₄): 549.2528, Calculated C₃₀H₃₇N₄O₄S⁺549.2530. MP: 227-230° C.

### Synthesis of 4-(6-methoxynaphthalen-2-yl)-2,6-dimethyl-1-(methyl(((6-((4R,5S)-5-methyl-2-oxoimidazolidin-4-yl)hexyl)oxy)carbonyl)amino)pyridin-1-ium trifluoroacetate (2c)

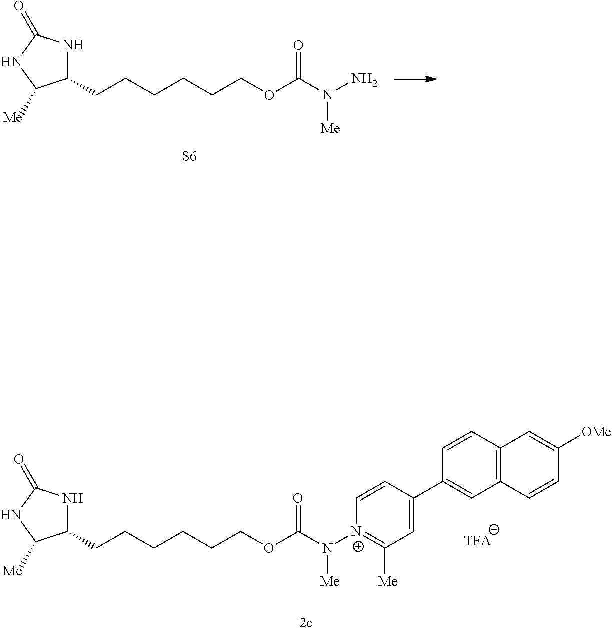 [SMILES: C[C@@H]1NC(=O)N[C@@H]1CCCCCCOC(=O)N(C)N ; "S6" ; COc1ccc2cc(-c3cc[n+](N(C)C(=O)OCCCCCC[C@H]4NC(=O)N[C@H]4C)c(C)c3)ccc2c1 ; "TFA"]

Pyridinium salt 2c was synthesized by adapting general procedure A as follows: To a flame dried round-bottom flask equipped with a stir bar was added 4-(6-methoxynaphthalen-2-yl)-2,6-dimethylpyrylium tetrafluoroborate (S2) (80 mg, 0.23 mmol), 6-((4R,5S)-5-methyl-2-oxoimidazolidin-4-yl)hexyl 1-methylhydrazine-1-carboxylate S6 (110 mg, 0.404 mmol), and ethanol (1.2 mL) and stirred for 24 hours at 60° C. The reaction mixture was then concentrated using a rotary evaporator. The resulting residue was dissolved in dichloromethane and washed with 10% fluoroboric acid (HBF₄, aqueous) (2×10 mL), saturated NaBF₄ (aqueous) (2×10 mL), dried over NaBF₄, filtered, and concentrated using a rotary evaporator. The resulting green solid was then sonicated in hexanes followed by decanting of the supernatant. This procedure was repeated 5 times and the resulting solid was dried under vacuum to obtain the tetrafluoroborate salt of 2c as a green solid (119 mg, 76%). The tetrafluoroborate salt of 2c was further purified by HPLC using general method B to yield the trifluoroacetate (X=TFA, CF₃CO₂⁻) salt of 2c. While the tetrafluoroborate salt (X=BF₄) of 2c was isolated in sufficient purity (>95%), the trifluoroacetate salt of 2c can possess superior water solubility compared to the tetrafluoroborate salt under the conditions tested.

4-(6-methoxynaphthalen-2-yl)-2,6-dimethyl-1-(methyl(((6-((4R,5S)-5-methyl-2-oxoimidazolidin-4-yl)hexyl)oxy)carbonyl)amino)pyridin-1-ium trifluoroacetate (2c) exists as a 1.1:1 mixture of rotamers at 298K. ¹H NMR (400 MHZ, CD₃OD) δ: 8.56-8.48 (s, 1H), 8.30-8.22 (s, 2H), 8.05-7.91 (m, 3H), 7.41-7.35 (s, 1H), 7.33-7.25 (s, 1H), 7.25-7.18 (s, 1H), 6.84 (s (br), 1H), 1H), 5.10 (s, br, 1H), 1H), 4.31 (t, J=6.3 Hz, 1H), 4.17 (t, J-6.4 Hz, 1H), 3.96 (s, 3H), 3.77 (m, 0.5H), 3.71-3.4 (m, 4.5H), 2.82-2.66 (m, 6H), 1.83-1.70 (m, 1H), 1.56-0.82 (m, 13H). ¹³C NMR (150 MHz, CD₃OD) δ: 165.4, 164.1, 164.0, 161.3, 161.3, 160.6 (q, J=60 Hz, 1C), 158.8, 158.7, 158.3, 158.3, 154.1, 152.7, 138.0, 138, 132.1, 132, 130.7, 130.7, 129.6, 129.5, 129.4, 129.1, 128.9, 125.5, 125.4, 125.3, 125.1, 125.1, 121.4, 121.3, 107.0, 69.5, 69.2, 56.7, 56.6, 56.4, 52.0, 51.9, 38.2, 37.2, 30.4, 30.3, 29.8, 29.5, 29.3, 28.9, 27, 26.3, 26.2, 19.5, 19.4, 15.8, 15.7. ¹⁹F NMR (376 MHZ, CD₃CN) δ: −76 8. FT-IR: 2929.03, 1698.4, 1613.8, 1326.9, 1193.0, 1023.2, 852.8, 519.8, 472.6. HR-MS (m/z) Found (M-CF₃CO₂): 519.3027, Calculated C₃₀H₃₉N₄O₄⁺519.2971. MP: 98-100° C.

### Synthesis of 1-((((6-azidohexyl)oxy)carbonyl)(methyl)amino)-4-(6-methoxynaphthalen-2-yl)-2,6-dimethylpyridin-1-ium tetrafluoroborate (2d)

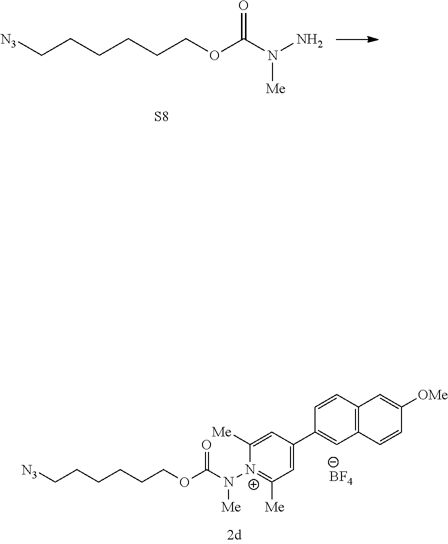

Pyridinium salt 2d was synthesized by adapting general procedure A as follows: To a flame dried round-bottom flask equipped with a stir bar was added 4-(6-methoxynaphthalen-2-yl)-2,6-dimethylpyrylium tetrafluoroborate (S2) (51 mg, 0.14 mmol, 1.0 equiv.), 6-azido-1-methylhydrazine-1-carboxylate (S8) (40 mg in a solution in 0.5 mL EtOH, 0.19 mmol, 1.3 equiv.), and ethanol (1.0 mL) and stirred for 3h at 65° C. The resulting mixture was then cooled down to room temperature and concentrated using a rotary evaporator. The yellow solid residue was then washed with diethyl ether. The solid residue was then dissolved in acetonitrile and filtered to remove any undissolved materials. The filtrate was concentrated using a rotary evaporator and dried under reduced pressure to yield pyridinium salt (1-((((6-azidohexyl)oxy)carbonyl)(methyl)amino)-4-(6-methoxynaphthalen-2-yl)-2,6-dimethylpyridin-1-ium tetrafluoroborate (2d))(62 mg, 78%) as yellow solid.

1-((((6-azidohexyl)oxy)carbonyl)(methyl)amino)-4-(6-methoxynaphthalen-2-yl)-2,6-dimethylpyridin-1-ium tetrafluoroborate (2d) exists as a 1.1:1 mixture of rotamers at 298 K. ¹H NMR (400 MHZ, CD₃OD) δ: 8.54 (d, J=3.80 Hz, 1H), 8.28 (d, J-3.64 Hz, 2H), 7.98-8.06 (m, 3H), 7.42 (s, 1H), 7.33 (d, J=9.40 Hz, 1H), 4.34 (t, J=6.60 Hz, 1H), 4.21 (t, J=6.34 Hz, 1H), 4.0 (s, 3H), 3.53 (s, 3H), 3.36 (t, J=6.80 Hz, 1H), 3.17 (t, J-6.80 Hz, 1H), 2.76 (s, 6H), 1.87-1.74 (m, 1H), 1.71-1.59 (m, 1H), 1.58-1.35 (m, 4H), 1.32-1.08 (m, 2H). ¹³C NMR (150 MHz, CD₃OD) δ: 160.3, 160.3, 157.8, 157.7, 157.3, 153.2, 151.7, 137.1, 137.0, 131.1, 131.0, 129.8, 129.7, 128.6, 128.6, 128.5, 128.4, 128.0, 127.9, 124.5, 124.4, 124.3, 124.2, 120.3, 120.3, 106.1, 68.4, 68.1, 55.4, 51.1, 50.8, 37.3, 36.2, 28.4, 28.2, 28.2, 27.9, 26.0, 25.7, 25.0, 24.9, 18.5, 18.4. ¹⁹F NMR (376 MHZ, CD₃CN) δ: −152.3—−152.4 (m). FT-IR: 2938.0, 2094.3, 1728.2, 1611.9, 1493.1, 1376.4, 1326.1, 1264.1, 1229.6, 1191.0, 1019.7, 854.8, 806.7, 758 8. HR-MS (m/z) Found (M-BF₄) 462.2444 m/z, Calculated C₂₆H₃₂N₅O₃⁺462.2500. MP: 63-68° C.

### D. Mechanistic Analysis

### D.1. Temporal Control Experiments

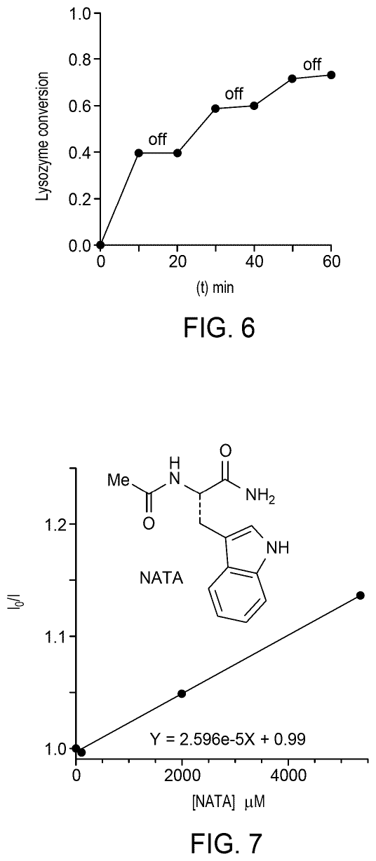

Labeling of lysozyme was performed as described above using the following irradiation time points: 0-10 min, lamp on; 10-20 min, lamp off; 20-30 min, lamp on; 30-40 min, lamp off; 40-50 min, lamp on; 50-60 min, lamp off. A 15 μL aliquot was taken every 10 minutes. The reaction was run with a 1 mL total volume so that aliquot removal would not significantly alter the reaction volume. The reaction was performed in duplicate and the average conversions are shown in FIG. 6.

The temporal control experiments indicate that the use of light as described herein can be used to achieve labeling of tryptophan residues in biological molecules and suppress chain-transfer-driven mechanisms.

D.2. Stern-Volmer Analysis of Pyridinium Salt 2a with N-Acetyl-L-Tryptophan

FIG. 7 is a Stern-Volmer plot of florescence quenching of pyridinium salt 2a with a small molecule tryptophan analog N-acetyltryptophanamide (NATA) according to at least one embodiment of the present disclosure. The Stern-Volmer plot was generated by excitation of a 100 μM solution of pyridinium salt 2a in 20 mM NH4OAc pH 6.9 in the presence of increasing concentrations of NATA. Analyses were performed at 375 nm excitation. The calculated K_{q}, assuming 11 (0.2548 ns) quenching, was determined to be about 1.019E5 M⁻¹s⁻¹. The calculated K_{q}, assuming τ₂ (3.399 ns) quenching, was determined to be about 7.638E3 M⁻¹s⁻¹. The Stern-Volmer plot (FIG. 7) of fluorescence quenching of pyridinium salt 2a with small molecule tryptophan analog NATA shows a measurable linear correlation, suggesting dynamic quenching of the excited state of pyridinium salt 2a with tryptophan.

### D.3. Absorption and Emission Spectra of Pyridinum Salt 2a

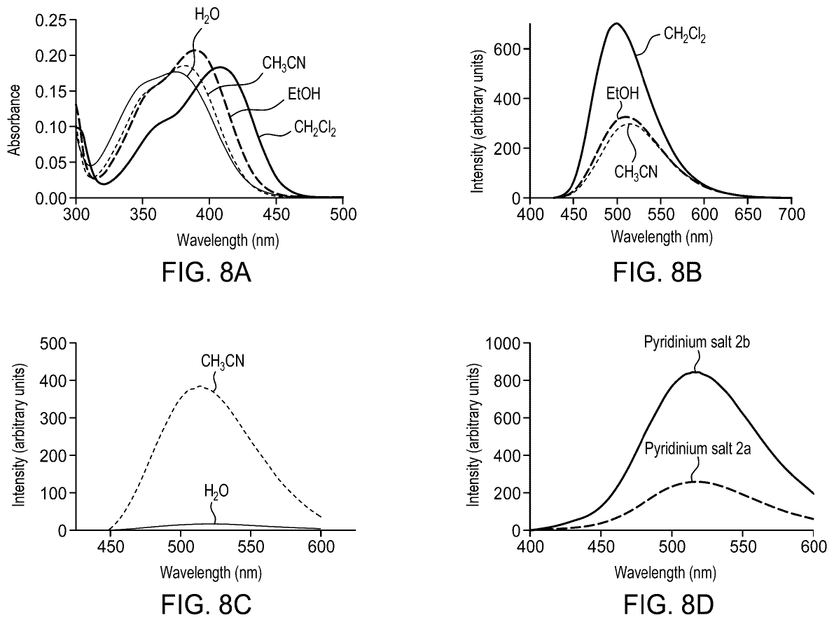

FIG. 8A shows absorption spectra of pyridinium salt 2a in water (H2O), acetonitrile (CH₃CN), ethanol (EtOH), and dichloromethane (CH₂Cl₂) according to at least one embodiment of the present disclosure. All spectra were recorded at a 10 μM concentration of pyridinium salt 2a in the desired solvent.

FIG. 8B shows emission spectra of pyridinium salt 2a in selected organic solvents. The emission spectra of pyridinium salt 2a in organic solvents were generated by excitation of a 10 μM solution of pyridinium salt 2a made with its respective solvent. Analyses were performed at 376 nm excitation with an emission slit width of 2.5 nm and an excitation slit width of 5 nm. The emission wavelength was gathered from 430 nm to 700 nm.

FIG. 8C shows emission spectra of pyridinium salt 2a in CH₃CN or H₂O according to at least one embodiment of the present disclosure. The emission spectra of pyridinium salt 2a were generated by excitation of a 1 μM solution of pyridinium salt 2a made with its respective solvent. Analyses were performed at 375 nm excitation with an emission slit width of 5 nm and an excitation slit width of 5 nm. The emission wavelength was gathered from 450 nm to 600 nm.

FIG. 8D shows emission spectra of pyridinium salt 2a versus pyridinium salt 2b according to at least one embodiment of the present disclosure. The emission spectra were generated by excitation of a 10 μM solution of either pyridinium salt 2a or pyridinium salt 2b in H2O. Analyses were performed at 375 nm excitation with an emission slit width of 5 nm and an excitation slit width of 5 nm. The emission wavelength was gathered from 400 nm to 600 nm.

### D.4. Lifetime Data for Pyridinium Salts 2a and 2b (Probes 2a and 2b)

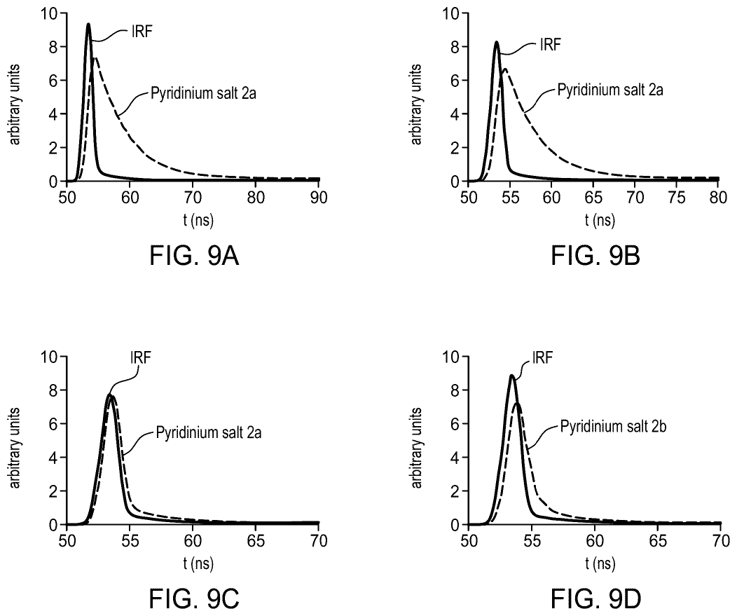

FIGS. 9A-9D show fluorescence decay spectra of pyridinium salts 2a and 2b in selected solvents according to at least one embodiment of the present disclosure. Specifically, FIG. 9A is a fluorescence decay spectrum of pyridinium salt 2a in 1,2-dichloroethane, FIG. 9B is a fluorescence decay spectrum of pyridinium salt 2a in CH₃CN, FIG. 9C is a fluorescence decay spectrum of pyridinium salt 2a in H2O, and FIG. 9D is a fluorescence decay spectrum of pyridinium salt 2b in H2O. The fluorescent decay spectra were generated with a 1-10 μM solution of pyridinium salt (10 μM for H₂O and 1 μM for other solvents) using a GG420 long pass filter and a 375 nm excitation wavelength. The instrument response factor was measured using a highly diluted solution of LUDOX AS-30 colloidal silica in water and a ND1.0 filter. The concentration of LUDOX and the emission slit width were adjusted until the intensity of the instrument response function (IRF) was similar to the fluorescent decay intensity. The procedure given in the EasyLife X User's Manual was followed.

Fluorescence lifetime measurements in 1,2-dichloroethane (FIG. 9A) and acetonitrile (FIG. 9B) both show one-phase exponential decays that correlate to τ₁=4.1 nanoseconds (ns) and τ₁=3.3 ns, respectively, suggesting a general decrease in lifetime as solvent polarity increases. However, two lifetimes are observed in water (τ₁=0.3 ns and τ₂=3.4 ns) as shown in FIG. 9C, with the second lifetime being of very weak intensity. Concurrently, the fluorescence intensity of pyridinium salt 2a decreases with increasing solvent polarity. These data suggest that (1) the excited state of pyridinium salt 2a is generally suppressed in aqueous systems compared to organic solvents and (2) pyridinium salt 2a* cannot defuse beyond 30 Å. The fluorescence lifetime experiments also show that pyridinium salt 2b has a shorter τ₂ (τ₁=0.5 ns and τ₂=2.8 ns) than pyridinium salt 2a, while a comparison of emission spectra reveal that pyridinium salt 2b has a higher fluorescence quantum yield (Φ_{F1}) than pyridinium salt 2a (relative Φ_{F1} of pyridinium salt 2b: pyridinium salt 2a=3.3) (FIG. 9D), suggesting a correlation between fluorescence quantum yield and kinetics.

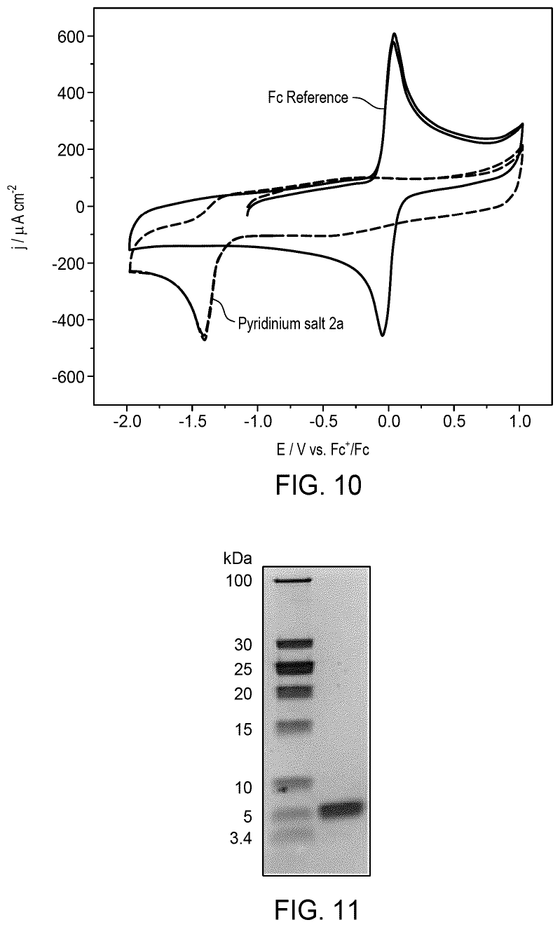

FIG. 10 is a cyclic voltammogram of pyridinium salt 2a in CH₃CN according to at least one embodiment of the present disclosure. Ferrocene (Fc) is also shown as a reference. The cyclic voltammogram was obtained at a sweep rate of 100 mV/s, after purging with N₂ for at least 3 min and maintaining a blanket of the gas over the cell throughout the measurement. A 3 mm diameter glassy C working electrode, platinum (Pt) coil counter, and a silver (Ag) wire quasireference electrode were used.

Overall, the temporal control experiments, Stern-Volmer analysis, absorption and emission spectra, lifetime data, and cyclic voltammetry data may suggest that the observed selectivity for tryptophan modification by pyridinium salt 2a may be driven by a synergistic combination of a short-lived excited state that lacks the persistence to enable kinetically slower processes and protein-pyridinium salt 2a pre-complexation via hydrophobic effects, as well as a kinetic preference for tryptophan over other redox-active residues.

### E. Purified Protein Modification Procedures & Non-Limiting Results

General Procedure B: Photochemical Irradiation of Peptides and Proteins: To a 2 mL Pyrex LC/MS vial (Thermo model #03-391-39) were sequentially added solutions containing the desired biomolecule, glutathione (GSH), pyridinium salt, and ammonium acetate (NH₄OAc). The resulting solution was diluted to a final volume of 500 μL and then sparged using nitrogen for a minimum of 30 minutes prior to irradiation. The actively sparging solution was then placed in an EvoluChem™ PhotoRedOx Box photoreactor that was equipped with a Kessil, PR160 427 nm LED Lamp and was then irradiated (100% lamp intensity) for the designated time at room temperature while maintaining active sparging. The resultant reaction mixture was then analyzed directly by LC/MS. Analysis of labeling outcomes using LC/MS was performed either by extracted ion chromatogram intensities (method A) or by integrating the area under the peaks associated with peptide content (method B). These values were totaled, and estimated percentages were assigned according to relative intensities of peaks or ion counts.

Labeling of Octreotide with Pyridinium Salt 2a (Octreotide-2a Conjugate). Labeling of Octreotide acetate (1 kDa) with the pyridinium salt 2a was performed by following general procedure B using the following solutions: Octreotide acetate (25 μL, 0.2 mM, 10 μM final concentration), pyridinium salt 2a (50 μL, 1.0 mM, 100 μM final concentration), ammonium acetate (NH4OAc, 50 μL, 200 mM, pH=6.9, 20 mM final concentration), glutathione (15 μL, 10 mM, 300 M final concentration), and water (360 μL) with a 100% intensity irradiation time of 30 minutes. The resulting reaction mixture was directly analyzed by LC/MS using method A to have proceeded with 88% conversion with mono labeling. Octreotide-2a conjugate (retention time (Tᵣ)=2.59-2.82 min).

A replicate reaction with Octreotide acetate (25 μL, 0.2 mM, 10 μM final concentration), pyridinium salt 2a (50 μL, 1.0 mM, 100 μM final concentration), glutathione (15 μL, 10 mM, 300 μM final concentration), NH₄OAc (50 μL, 200 mM, pH 6.9, 20 mM final concentration), and water (360 μL) proceeded with 93% conversion with mono labeling.

Labeling of Leuprolide with Pyridinium Salt 2a (Leuprolide-2a Conjugate). Labeling of Leuprolide (1.2 kDa) with the pyridinium salt 2a was performed by following general procedure B using the following solutions: leuprolide (13 μL, 387 μM, 10 μM final concentration), pyridinium salt 2a (34 μL, 1.483 mM, 100 μM final concentration), NH4+OAc (50 μL, 200 mM, pH=6.9, 20 mM final concentration), glutathione (50 μL, 3 mM, 300 μM final concentration), and water (353 μL) with a 100% intensity irradiation time of 30 minutes. The reaction mixture was analyzed directly by LC/MS using method B and was estimated to have proceeded with 91% conversion, a >20:1 mono:di labeled ratio, and 4% of +1 label+glutathionylation. Leuprolide-2a conjugate (retention time (Tᵣ)=5.48-8.31 min).

A replicate reaction with Leuprolide (25 μL, 199 μM, 10 μM final concentration), pyridinium salt 2a (34 μL, 1.483 mM, 100 μM final concentration), NH₄OAc (50 μL, 200 mM, pH=6.9, 20 mM final concentration), glutathione (50 μL, 3 mM, 300 μM final concentration), and water (341 μL) proceeded in 91% conversion, a >20:1 mono:di labeled ratio, and 4% of +1 label+glutathionylation.

Preparative Scale Labeling of Leuprolide with Pyridinium Salt 2a (Leuprolide-2a Conjugate). Preparative scale labeling of Leuprolide (1.2 kDa) with the pyridinium salt 2a was performed using the following procedure: to a 10 mL pear-shaped flask equipped with a septum were sequentially added Leuprolide (424 μL, 4720 μM, 400 μM final concentration), pyridinium salt 2a (3354 μL, 2.236 mM, 1.5 mM final concentration), NH4OAc (500 μL, 200 mM, pH=6.9, 20 mM final concentration), glutathione (400 μL, 50 mM, 4 mM final concentration), and water (322 μL) to give a final volume of 5 mL. The homogeneous solution was then degassed for one hour using nitrogen and lowered into the EvoluChem™ PhotoRedOx Box. The solution was irradiated for 2 hours. The reaction mixture was then purified directly by HPLC using method A and analyzed using quantitative NMR in 20 mM deuterated ammonium acetate to obtain a 40% isolated yield. ¹H-NMR of the Leuprolide-2a conjugate at 60° C. in 20 mM ammonium acetate-D7 buffered deuterium oxide (D20) with a dimethylformamide (DMF) internal standard confirmed the conjugate.

Labeling of Exenatide with Pyridinium Salt 2a (Exenatide-2a Conjugate). Labeling of Exenatide (4.2 kDa) was performed by following general procedure B using the following solutions: Exenatide (24.6 μL, 0.204 mM solution in H2O, final concentration 10 μM) was labeled with the pyridinium salt 2a (100 μL, 1.0 mM solution in H2O, final concentration 200 μM), NH₄OAc (50 μL, 200 mM in H2O, final concentration 20 mM, pH 6.9), glutathione (40 μL, 10 mM solution in H2O, final concentration 800 μM), and diluted with water (285.4 μL) in 60 min irradiation period under 100% intensity irradiation. The resulting reaction mixture was directly analyzed by LC/MS using method A and was estimated to have proceeded in 70% conversion. Exenatide-2a conjugate (retention time (Tᵣ)=3.24-3.56 min).

A replicate reaction with exenatide (24.6 μL, 0.204 mM solution in H2O, final concentration 10 μM) was labeled with pyridinium salt 2a (100 μL, 1.0 mM solution in H2O, final concentration 200 μM), NH₄OAc (50 μL, 200 mM in H2O, final concentration 20 mM, pH 6.9), glutathione (40 μL, 10 mM solution in H2O, final concentration 800 μM), and diluted with water (285.4 μL) in 60 min irradiation period under UV B (427 nm, Intensity 100) light processed with 69% conversion.

Labeling of Thioredoxin with Pyridinium Salt 2a (Thioredoxin-2a Conjugate). Labeling of thioredoxin (11.6 kDa) with the pyridinium salt 2a was performed by following general procedure B using the following solutions: thioredoxin (49 μL, 103 μM, 10 μM final concentration), pyridinium salt 2a (88 μL, 1.141 mM, 200 μM final concentration), NH₄OAc (50 μL, 200 mM, pH 6.9, 20 mM final concentration), glutathione (50 μL, 3 mM, 300 μM final concentration), and water (263 μL) with a 100% intensity irradiation time of 60 minutes. The reaction mixture was directly analyzed by LC/MS using method A and was estimated to have proceeded with 93% conversion and a 14.5:1 mono:di labeled ratio and 3% glutathionylation of all protein content. Thioredoxin-2a conjugate (retention time (Tᵣ)=4.36-5.35 min).

A replicate reaction with thioredoxin (18 μL, 280 μM, 10 UM final concentration), pyridinium salt 2a (84 μL, 1.187 mM, 200 μM final concentration), NH₄OAc (50 μL, 200 mM, pH 6.9, 20 mM final concentration), glutathione (50 μL, 3 mM, 300 μM final concentration), and water (298 μL) proceeded in 87% conversion with a >20:1: mono:di labeled ratio and trace glutathionylation of all protein content. No mass change was observed following treatment of the reaction mixture with excess N-methylmaleimide, suggesting that the disulfide bridge in the protein was oxidized during the reaction.

Labeling of β₂-Microglobulin with Pyridinium Salt 2a (B2-Microglobulin-2a Conjugate). Labeling of β₂-Microglobulin (11.7 kDa) with the pyridinium salt 2a was performed by following general procedure B using the following solutions: B2-Microglobulin (28 μL, 181 μM, 10 μM final concentration), pyridinium salt 2a (67 μL, 1.483 mM, 200 μM final concentration), NH₄OAc (50 μL, 200 mM, pH 6.9, 20 mM final concentration), glutathione (167 μL, 3 mM, 1 mM final concentration) and water (188 μL) with a 100% intensity irradiation time of 30 minutes. The reaction mixture was directly analyzed by LC/MS using method A and was estimated to have proceeded with 88% conversion with a >20:1 mono:di labeled ratio and 7% glutathionylation of all unlabeled and labeled protein content. β₂-Microglobulin-2a conjugate (retention time (Tᵣ)=3.26-4.70 min).

A replicate reaction with B2-Microglobulin (28 μL, 181 μM, 10 μM final concentration), pyridinium salt 2a (67 μL, 1.483 mM, 200 μM final concentration), NH₄OAc (50 μL, 200 mM, pH 6.9, 20 mM final concentration), glutathione (167 μL, 3 mM, 1 mM final concentration), and water (188 μL) proceeded in 88% conversion with a >20:1 mono:di labeled ratio and 7% glutathionylation of all unlabeled and labeled protein content.

Labeling of Chymotrypsinogen with pyridinium salt 2a (Chymotrypsinogen-2a Conjugate). Labeling of chymotrypsinogen (25 kDa) with the pyridinium salt 2a was performed by following general procedure B using the following solutions: chymotrypsinogen (22 μL, 230 μM, 10 μM final concentration), pyridinium salt 2a (66 μL, 1.521 mM, 200 μM final concentration), NH₄OAc (50 μL, 200 mM, pH 6.9, 20 mM final concentration), glutathione (50 μL, 3 mM, 300 μM final concentration), and water (312 μL) with a 100% intensity irradiation time of 60 minutes. The reaction mixture was directly analyzed by LC/MS using method A and was estimated to have proceeded with 90% conversion with a 20.7:8.3:1 mono:di:tri labeled ratio and 4% glutathionylation of all unlabeled and labeled protein content. Chymotrypsinogen-2a conjugate (retention time (Tᵣ)=4.07-5.38 min).

A replicate reaction with chymotrypsinogen (22 μL, 230 μM, 10 μM final concentration), pyridinium salt 2a (66 μL, 1.521 mM, 200 μM final concentration), NH₄OAc (50 μL, 200 mM, pH 6.9, 20 mM final concentration), glutathione (50 μL, 3 mM, 300 μM final concentration), and water (312 μL) proceeded in 89% conversion with a 15:6.5:1: mono:di labeled ratio and trace glutathionylation of all unlabeled and labeled protein content.

Labeling of Bovine Carbonic Anhydrase II with Pyridinium Salt 2a (Bovine Carbonic Anhydrase II-2a Conjugate). Labeling of bovine carbonic anhydrase II (29 kDa) with the pyridinium salt 2a was performed by following general procedure B using the following solutions: carbonic anhydrase II (58 μL, 86 μM, 10 μM final concentration), 2a (89 μL, 1.118 mM, 200 μM final concentration), NH₄OAc (50 μL, 200 mM, pH 6.9, 20 mM final concentration), glutathione (50 μL, 1 mM, 300 μM final concentration), and water (253 μL) with a 100% intensity irradiation time of 60 minutes. The reaction mixture was directly analyzed by LC/MS using method A and was estimated to have proceeded with 34% conversion and a 4.5:1 mono:di labeled ratio. No glutathionylation was observed.

A replicate reaction with carbonic anhydrase II (50 μL, 100 μM, 10 μM final concentration), pyridinium salt 2a (66 μL, 1.521 mM, 200 μM final concentration), NH₄OAc (50 μL, 200 mM, pH 6.9, 20 mM final concentration), glutathione (50 μL, 3 mM, 300 μM final concentration) and water (284 μL) proceeded in 33% conversion with a 4.5:1: mono:di labeled ratio. No glutathionylation was observed. LC/MS was performed on the bovine carbonic anhydrase II-2a conjugate (retention time (Tᵣ)=4.15-5.69 min).

Labeling of Lysozyme with Pyridinium Salt 2a (Lysozyme-2a Conjugate). Labeling of lysozyme (14.3 kDa) with the pyridinium salt 2a was performed by following general procedure B using the following solutions: lysozyme (27 μL, 182 μM, 10 μM final concentration), pyridinium salt 2a (200 μL, 500 μM, 200 μM final concentration), NH4+OAc (100 μL, 100 mM, pH=6.9, 20 mM final concentration), glutathione (15 μL, 10 mM, 300 μM final concentration), and water (158 μL) with a 100% intensity irradiation time of 60 minutes. The reaction mixture was analyzed directly by LC/MS using method A and was estimated to have proceeded in >95% conversion and a 10.9:1 mono:di labeled ratio and 10% glutathionylation of all unlabeled and labeled protein content was observed.

A replicate reaction with lysozyme (19 μL, 262 μM, 10 μM final concentration), pyridinium salt 2a (66 μL, 1.521 mM, 200 μM final concentration), glutathione (50 μL, 3 mM, 300 μM final concentration), NH₄OAc (50 μL, 200 mM, pH 6.9, 20 mM final concentration), and water (315 μL) proceeded with 94% conversion, and a 6.6:1 mono:di labeled ratio and 10% glutathionylation of all unlabeled and labeled protein content was observed. LC/MS was performed on the Lysozyme-2a conjugate (retention time (Tᵣ)=2.83-5.53 min).

Labeling of Lysozyme with Pyridinium Salt 2b (Lysozyme-2b Conjugate). Labeling of lysozyme (14.3 kDa) with the pyridinium salt 2b was performed by following general procedure B using the following solutions: lysozyme (19 μL, 262 μM, 10 μM final concentration), 2b (181 μL, 554 μM, 200 μM final concentration), NH₄OAc (50 μL, 200 mM, pH=6.9, 20 mM final concentration), glutathione (50 μL, 3 mM, 300 μM final concentration), and water (200 μL) with a 100% intensity irradiation time of 10 minutes. The reaction mixture was analyzed directly by LC/MS using method A and was estimated to have proceeded with 83% conversion. Partial oxidation at biotin obfuscated estimation of both oxidation and glutathionylation. Lysozyme-2b conjugate (retention time (Tᵣ)=2.80-5.17 min).

A replicate reaction with lysozyme (19 μL, 262 μM, 10 μM final concentration), pyridinium salt 2b (181 μL, 554 μM, 200 μM final concentration), glutathione (50 μL, 3 mM, 300 μM final concentration), NH4+OAc (50 μL, 200 mM, pH 6.9, 20 mM final concentration), and water (200 μL) proceeded in 80% conversion.

Labeling of Lysozyme with Pyridinium Salt 2c (Lysozyme-2c Conjugate). Labeling of lysozyme (14.3 kDa) with the pyridinium salt 2c was performed by following general procedure B using the following solutions: lysozyme (19 μL, 262 μM, 10 μM final concentration), pyridinium salt 2c(367 μL, 680 μM, 500 μM final concentration), NH₄OAc (50 μL, 200 mM, pH=6.9, 20 mM final concentration), glutathione (50 μL, 3 mM, 300 μM final concentration), and water (14 μL) with a 100% intensity irradiation time of 10 minutes. The reaction mixture was analyzed directly by LC/MS using method A and was estimated to have proceeded with 85% conversion and a 3.7:1 mono:di labeled ratio and 10% glutathionylation of all protein content was observed. Lysozyme-2c conjugate (retention time (Tᵣ)=3.11-5.07 min).

A replicate reaction with lysozyme (19 μL, 262 μM, 10 μM final concentration), pyridinium salt2c (367 μL, 680 μM, 500 μM final concentration), NH₄OAc (50 μL, 200 mM, pH=6.9, 20 mM final concentration), glutathione (50 μL, 3 mM, 300 μM final concentration), and water (14 μL) proceeded with 86% conversion with a 3.5:1 mono:di labeled ratio and 10% glutathionylation of all protein content was observed.

Labeling of Lysozyme with Pyridinium Salt 2d (Lysozyme-2d Conjugate). Labeling of lysozyme (14.3 kDa) with the pyridinium salt 2d was performed by following general procedure B using the following solutions: Lysozyme (16.8 μL, 0.297 mM solution in H2O, final concentration 10 M), pyridinium salt 2d (91.8 μL, 1.09 mM solution in CH₃CN/H2O (2:3), final concentration 200 μM), NH₄OAc (50 μL, 200 mM in H₂O, final concentration 20 mM, pH 6.9), glutathione (15 μL, 10 mMsolution in H2O, final concentration 300 μM), and water (326.4 μL) with a 100% intensity irradiation time of 20 minutes. The resulting reaction mixture was directly analyzed by LC/MS using method A and was estimated 85% conversion with >20:1 mono:di labeling and 3% glutathionylation of all unlabeled and labeled protein content. Lysozyme-2d conjugate (retention time (Tᵣ)=2.27-4.68 min).

A replicate reaction with lysozyme (16.8 μL, 0.297 mM solution in H2O, final concentration 10 μM) was labeled with pyridinium salt 2d (91.8 μL, 1.09 mM solution in CH₃CN/H2O (2:3), final concentration 200 μM), NH₄OAc (50 μL, 200 mM in H2O, final concentration 20 mM, pH 6.9), glutathione (15 μL, 10 mM solution in H2O, final concentration 300 μM) and diluted with water (326.4 μL) proceeded with 84% conversion with a >20:1 mono:di labeling ratio and 2% glutathionylation of all unlabeled and labeled protein content.

MS-MS Analysis of Labeled Protein Conjugates. Labeled proteins were digested by Protocol A or Protocol B.

General Trypsin Digest Protocol A: The labeled protein solution was concentrated using an Amicon 10 kDa filter. This concentrate was diluted with urea (240 μL, 10 M, 6 M final concentration), tris-buffer (100 μL, 50 mM, pH 7.9, 12.5 mM final concentration), and tris(2-carboxyethyl)phosphine (TCEP, 20 μL, 100 mM, 5 mM final concentration). This solution was incubated for 10 minutes at 50° C. N-methylmaleimide (40 μL, 150 mM, 14.2 mM final concentration) was added directly and again incubated 30 minutes at room temperature. The reduced, Cys-capped labeled protein was then concentrated using an Amicon 10 kDa filter (final volume 40 μL). The concentrate was then diluted with urea (40 μL, 10 M, 2 M final concentration), tris base buffer (135 μL, 50 mM, pH 7.9, 33.8 mM final concentration), calcium chloride (CaCl₂), 5 μL, 50 mM, 1.25 mM final concentration), and trypsin protease (1 μL, 0.5 μg/μL, 0.002 μg/μL final concentration) and incubated at 37° C. for 20 hours. Formic acid (2.5 μL) was then transferred in by a syringe and briefly agitated prior to analysis by LC/MS and MS/MS using method C.

General Trypsin Digest Protocol B: For non-cystine bridged labeled proteins, a standard trypsin digest was modified as follows. The labeled protein solution was concentrated using an Amicon 10 kDa filter. This concentrate was diluted with urea (240 μL, 10 M, 6 M final concentration), tris base buffer (150 μL, 50 mM, pH 8.4, 18.75 mM final concentration), CaCl₂) (10 μL, 50 mM, 1.25 mM final concentration), and trypsin protease (1 μL, 0.5 μg/μL, 0.002 μg/μL final concentration) and incubated at 37° C. for 24 hours. The digested mixture was analyzed directly by LC/MS and MS/MS using method C. Octreotide-2a Conjugate. Tandem mass spectra were taken by stirring the reaction mixture with excess of TCEP for breaking the S—S bond in octreotide for at least 30 minutes after photochemical reaction. The reduced product was analyzed by LC/MS-MS by selecting m/z=1108.4 for collision-induced dissociation (Collision energy=35 V). Mass spectrum peaks (b4+), (b5+), (b6+), (b7+), (y6+), and (y4+) confirmed the tagged/modified tryptophan product and are shown in Table D1. “AA #” refers to the amino acid number.

TABLE D1

| AA # | b(+) | | AA # | y(+) |
|---|---|---|---|---|
| 1 | 148.1 | | 8 | — |
| 2 | 251.1 | | 7 | 961.4 |
| 3 | 398.2 | | 6 | 858.7 (y6+) |
| 4 | 671.5 | (b4+) | 5 | 711.3 |
| 5 | 799.5 | (b5+) | 4 | 438.2 (y4+) |
| 6 | 900.6 | (b6+) | 3 | 310.1 |
| 7 | 1003.7 | (b7+) | 2 | 209.1 |

| | | | |
|---|---|---|---|
| 8 | — | 1 | 106.1 |

Leuprolide-2a Conjugate. Tandem MS data was obtained by LC/MS, by selecting m/z=1296.6 for collision-induced dissociation (Collision energy=35 V). Mass spectrum peaks (b2+), (b3+), (b4+), (b5+), (b6+), (b7+), (b8+), (b8++), (y2+), (y3+), (y4+), (y5+), (y6+), (y7+), and (y7++) confirmed the tagged/modified tryptophan product and are shown in Table D2. “AA #” refers to the amino acid number.

TABLE D2

| AA # | b(+) | | b(++) | AA # | y(+) | | y(++) |
|---|---|---|---|---|---|---|---|
| 1 | 112.0 | | 56.5 | 9 | — | | — |
| 2 | 249.1 | (b2+) | 125.1 | 8 | 1185.6 | | 593.3 |
| 3 | 522.2 | (b3+) | 261.6 | 7 | 1048.5 | (y7+) | 524.8 (y7++) |
| 4 | 609.2 | (b4+) | 305.1 | 6 | 775.4 | (y6+) | 388.2 |
| 5 | 772.3 | (b5+) | 386.6 | 5 | 688.4 | (y5+) | 344.7 |
| 6 | 885.4 | (b6+) | 443.2 | 4 | 525.3 | (y4+) | 263.2 |
| 7 | 998.4 | (b7+) | 499.7 | 3 | 412.2 | (y3+) | 206.6 |
| 8 | 1154.6 | (b8+) | 577.8 (b8++) | 2 | 299.2 | (y2+) | 150.1 |

| | | | | | |
|---|---|---|---|---|---|
| 9 | — | — | 1 | 143.1 | 72.0 |

Exenatide-2a Conjugate. Exenatide was digested according to General Protocol B. Briefly, an aliquot of 25 μL (0.2 mM) exenatide solution was diluted with urea (240 μL, 10M) tris base buffer (100 μL, 50 mM, pH 7.8), CaCl₂) (10 μL, 100 mM) and trypsin protease (1 μL, 0.5 μg/μL) and incubated at 37° C. for 15 h. The digested mixture was analyzed directly by LC-MS and MS/MS. Tandem MS data was obtained by LC/MS by selecting m/z=1035.5 for collision-induced dissociation (Collision energy=35 V). Mass spectrum peaks (b4+), (b5+), (b6+), (b6++), (y3+), (y4+), and (y5+), confirmed the tagged/modified tryptophan product and are shown in Table D3. “AA #” refers to the amino acid number.

TABLE D3

| AA # | b(+) | b(++) | AA # | y(+) |
|---|---|---|---|---|
| 1 | 114.1 | 57.5 | 7 | — |
| 2 | 261.2 | 131.1 | 6 | 922.5 |
| 3 | 374.2 | 187.6 | 5 | 775.7 (y5+) |
| 4 | 503.5 (b4+) | 252.1 | 4 | 662.6 (y4+) |
| 5 | 776.6 (b5+) | 388.7 | 3 | 533.6 (y3+) |
| 6 | 889.7 (b6+) | 444.8 (b6++) | 2 | 260.2 |
| 7 | — | — | 1 | 147.1 |

Thioredoxin-2a Conjugate. Tandem MS data was obtained after analysis of the Protocol A digestion by LC/MS by selecting m/z=1146.2 for collision-induced dissociation (Collision energy=35 V). Mass spectrum peaks (b5+), (b7+), (b8+), (b9+), (b10+), (b12+), (b13+), (b14+), (b15+), (y2+), (y3+), (y4+), (y5+), (y6+), (y7+), (y8+), (y9+), (y10+), (y11+), (y12+), and (y13+) confirmed the tagged/modified tryptophan product and are shown in Table D4. “AA #” refers to the amino acid number.

TABLE D4

| AA # | b(+) | | AA # | y(+) | |
|---|---|---|---|---|---|
| 1 | 72.0 | | 18 | — | |
| 2 | 187.1 | | 17 | 2219.1 | |
| 3 | 244.1 | | 16 | 2104.1 | |
| 4 | 315.1 | | 15 | 2047.1 | |
| 5 | 428.2 | (b5+) | 14 | 1976.1 | |
| 6 | 541.3 | | 13 | 1863.0 | (y13+) |
| 7 | 640.4 | (b7+) | 12 | 1749.9 | (y12+) |
| 8 | 755.4 | (b8+) | 11 | 1650.8 | (y11+) |
| 9 | 902.5 | (b9+) | 10 | 1535.8 | (y10+) |
| 10 | 1088.5 | (b10+) | 9 | 1388.7 | (y9+) |
| 11 | 1159.6 | | 8 | 1202.6 | (y8+) |
| 12 | 1288.6 | (b12+) | 7 | 1131.6 | (y7+) |
| 13 | 1561.8 | (b13+) | 6 | 1002.6 | (y6+) |
| 14 | 1775.9 | (b14+) | 5 | 729.4 | (y5+) |
| 15 | 1832.9 | (b15+) | 4 | 515.3 | (y4+) |

| | | | | |
|---|---|---|---|---|
| 16 | 1930.0 | 3 | 458.3 | (y3+) |
| 17 | 2144.1 | 2 | 361.2 | (y2+) |

| | | | |
|---|---|---|---|
| 18 | — | 1 | 147.1 |

Chymotrypsinogen-2a Conjugate (W215). Tandem MS data was obtained after analysis of the Protocol A digestion (modified to exclude the tris buffer) by LC/MS by selecting m/z=1478.8 for collision-induced dissociation (Collision energy=35 V). Mass spectrum peaks (b7+), (b9+), (b10+), (b11+), (b12+), (b13+), (b16+), (b11++), (b22++), (y4+), (y5+), (y6+), (y7+), (y8+), (y9+), (y10+), (y11+), (y12+), (y13+), (y14+), (y15+), (y16+), (y17+), (y9++), (y14++), (y20++), (y21++), and (y24++) the tagged/modified tryptophan (W215) product and are shown in Table D5. “AA #” refers to the amino acid number.

TABLE D5

| AA # | b(+) | | b(++) | AA # | y(+) | | y(++) |
|---|---|---|---|---|---|---|---|
| 1 | 115.1 | | 58.0 | 27 | — | | — |
| 2 | 172.1 | | 86.5 | 26 | 2841.4 | | 1421.2 |
| 3 | 243.1 | | 122.1 | 25 | 2784.3 | | 1392.7 |
| 4 | 429.2 | | 215.1 | 24 | 2713.3 | | 1357.2 (y24++) |
| 5 | 530.2 | | 265.6 | 23 | 2527.2 | | 1264.1 |
| 6 | 643.3 | | 322.2 | 22 | 2426.2 | | 1213.6 |
| 7 | 742.4 | (b7+) | 371.7 | 21 | 2313.1 | | 1157.1 (y21++) |
| 8 | 799.4 | | 400.2 | 20 | 2214.0 | | 1107.5 (y20++) |
| 9 | 912.5 | (b9+) | 456.8 | 19 | 2157.0 | | 1079.0 |
| 10 | 1011.6 | (b10+) | 506.3 | 18 | 2043.9 | | 1022.5 |
| 11 | 1098.6 | (b11+) | 549.8 (b11++) | 17 | 1944.9 | (y17+) | 972.9 |
| 12 | 1371.8 | (b12+) | 686.4 | 16 | 1857.8 | (y16+) | 929.4 |
| 13 | 1428.8 | (b13+) | 714.9 | 15 | 1584.7 | (y15+) | 792.8 |
| 14 | 1515.8 | | 758.4 | 14 | 1527.6 | (y14+) | 764.3 (y14++) |
| 15 | 1602.8 | | 801.9 | 13 | 1440.6 | (y13+) | 720.8 |
| 16 | 1703.9 | (b16+) | 852.4 | 12 | 1353.6 | (y12+) | 677.3 |

| | | | | | | |
|---|---|---|---|---|---|---|
| 17 | 1917.9 | 959.5 | 11 | 1252.5 | (y11+) | 626.8 |
| 18 | 2004.9 | 1003.0 | 10 | 1038.5 | (y10+) | 519.8 |
| 19 | 2106.0 | 1053.5 | 9 | 951.5 | (y9+) | 476.2 (y9++) |
| 20 | 2193.0 | 1097.0 | 8 | 850.4 | (y8+) | 425.7 |
| 21 | 2294.1 | 1147.5 | 7 | 763.4 | (y7+) | 382.2 |
| 22 | 2391.1 | 1196.1 (b22++) | 6 | 662.4 | (y6+) | 331.7 |
| 23 | 2448.1 | 1224.6 | 5 | 565.3 | (y5+) | 283.2 |
| 24 | 2547.2 | 1274.1 | 4 | 508.3 | (y4+) | 254.6 |

| | | | | | |
|---|---|---|---|---|---|
| 25 | 2710.3 | 1355.6 | 3 | 409.2 | 205.1 |
| 26 | 2781.3 | 1391.2 | 2 | 246.2 | 123.6 |
| 27 | — | — | 1 | 175.1 | 88.1 |

Chymotrypsinogen-2a Conjugate (W237). Tandem MS data was obtained after analysis of the Protocol A digestion (modified to exclude the tris buffer) by LC/MS by selecting m/z=858.7 for collision-induced dissociation (Collision energy=35 V). Mass spectrum peaks (b4+), (b5+), (b6+), (b8+), (b9+), (b11+), (b12+), (b13+), (b14+), (b8++), (b12++), (b13++), (b14++), (y3+), (y4+), (y6+), (y8+), (y9+), (y10+), (y11+), (y10++), (y12++), and (y13++) confirmed the tagged/modified tryptophan (W237) product and are shown in Table D6. “AA #” refers to the amino acid number.

TABLE D6

| AA # | b(+) | | b(++) | | AA # | y(+) | | y(++) |
|---|---|---|---|---|---|---|---|---|
| 1 | 100.1 | | 50.5 | | 15 | — | | — |
| 2 | 201.1 | | 101.1 | | 14 | 1615.9 | | 808.4 |
| 3 | 272.2 | | 136.6 | | 13 | 1514.8 | | 757.9 (y13++) |
| 4 | 385.2 | (b4+) | 193.1 | | 12 | 1443.8 | | 722.4 (y12++) |
| 5 | 484.3 | (b5+) | 242.7 | | 11 | 1330.7 | (y11+) | 665.9 |
| 6 | 598.4 | (b6+) | 299.7 | | 10 | 1231.7 | (y10+) | 616.3 (y10++) |
| 7 | 871.5 | | 436.3 | | 9 | 1117.6 | (y9+) | 559.3 |
| 8 | 970.6 | (b8+) | 485.8 | (b8++) | 8 | 844.5 | (y8+) | 422.7 |
| 9 | 1098.6 | (b9+) | 549.8 | | 7 | 745.4 | | 373.2 |
| 10 | 1226.7 | | 613.9 | | 6 | 617.3 | (y6+) | 309.2 |
| 11 | 1327.7 | (b11+) | 664.4 | | 5 | 489.3 | | 245.1 |
| 12 | 1440.8 | (b12+) | 720.9 | (b12++) | 4 | 388.2 | (y4+) | 194.6 |
| 13 | 1511.9 | (b13+) | 756.4 | (b13++) | 3 | 275.1 | (y3+) | 138.1 |

| | | | | | | | |
|---|---|---|---|---|---|---|---|
| 14 | 1582.9 | (b14+) | 792.0 | (b14++) | 2 | 204.1 | 102.6 |

| | | | | | |
|---|---|---|---|---|---|
| 15 | — | — | 1 | 133.1 | 67.0 |

Lysozyme-2a Conjugate. Tandem MS data was obtained after analysis of the Protocol A digestion by LC/MS by selecting m/z=957.6 for collision-induced dissociation (Collision energy=35 V). Mass spectrum peaks (b3+), (b4+), (b5+), (b6+), (b7+), (b13+), (b14+), (b15+), (b16+), (b8++), (b12++), (b13++), (b14++), (y4+), (y5+), (y6+), (y7++), (y8++), (y9++), (y10++), (y11++), (y12++), (y13++), (y15++), (y16++), (y17++), (y18++), (y19++), (y20++), (y21++), and (y22++) confirmed the tagged/modified tryptophan product and are shown in Table D7. “AA #” refers to the amino acid number.

TABLE D7

| AA # | b(+) | | AA # | y(+) | y(++ | |
|---|---|---|---|---|---|---|
| 1 | 115.1 | | 23 | — | — | |
| 2 | 216.1 | | 22 | 2755.2 | 1378.1 | (y22++) |
| 3 | 331.1 | (b3+) | 21 | 2654.1 | 1327.6 | (y21++) |
| 4 | 388.1 | (b4+) | 20 | 2539.1 | 1270.0 | (y20++) |
| 5 | 475.2 | (b5+) | 19 | 2482.1 | 1241.5 | (y19++) |
| 6 | 576.2 | (b6+) | 18 | 2395.0 | 1198.0 | (y18++) |
| 7 | 691.3 | (b7+) | 17 | 2294.0 | 1147.5 | (y17++) |
| 8 | 854.3 | | 16 | 2178.9 | 1090.0 | (y16++) |
| 9 | 911.3 | | 15 | 2015.9 | 1008.4 | (y15++) |
| 10 | 1024.4 | | 14 | 1958.9 | 979.9 | |
| 11 | 1137.5 | | 13 | 1845.8 | 923.4 | (y13++) |
| 12 | 1265.6 | | 12 | 1732.7 | 866.9 | (y12++) |
| 13 | 1378.6 | (b13+) | 11 | 1604.6 | 802.8 | (y11++) |
| 14 | 1492.7 | (b14+) | 10 | 1491.6 | 746.3 | (y10++) |
| 15 | 1579.7 | (b15+) | 9 | 1377.5 | 689.3 | (y9++) |
| 16 | 1735.8 | (b16+) | 8 | 1290.5 | 645.7 | (y8++) |

| | | | | | |
|---|---|---|---|---|---|
| 17 | 2008.9 | 7 | 1134.4 | 567.7 | (y7++) |

| | | | | |
|---|---|---|---|---|
| 18 | 2195.0 | 6 | 861.3 (y6+) | 431.2 |
| 19 | 2409.0 | 5 | 675.2 (y5+) | 338.1 |
| 20 | 2523.0 | 4 | 461.2 (y4+) | 231.1 |
| 21 | 2638.1 | 3 | 347.2 | 174.1 |
| 22 | 2695.1 | 2 | 232.1 | 116.6 |
| 23 | — | 1 | 175.1 | 88.1 |

Carbonic Anhydrase II-2a Conjugate. Tandem MS data was obtained after analysis of the Protocol B digestion by LC/MS by selecting m/z=1070.8 for collision-induced dissociation (Collision energy=35 V). Mass spectrum peaks (b3+), (b4+), (b6+), (b7+), (b14+), (b15+), (b16+), (b8++), (b9++), (b10++), (b12++), (y3+), (y4+), (y5+), (y6+), (y7+), (y8+), (y13+), (y5++), (y9++), (y10++), (y11++), (y12++), (y13++), (y14++), (y15++), (y18++), (y19++), (y20++), (y21++), (y22++), (y23++), (y24++), (y25++), and (y26++) confirmed the tagged/modified tryptophan product and are shown in Table D8. “AA #” refers to the amino acid number.

TABLE D8

| AA # | b(+) | | b(++) | AA # | y(+) | y(+) | |
|---|---|---|---|---|---|---|---|
| 1 | 102.1 | | 51.5 | 27 | | — | |
| 2 | 215.1 | | 108.1 | 26 | 3108.7 | 1554.8 | (y26++) |
| 3 | 329.2 | (b3+) | 165.1 | 25 | 2995.6 | 1498.3 | (y25++) |
| 4 | 476.3 | (b4+) | 238.6 | 24 | 2881.5 | 1441.3 | (y24++) |
| 5 | 590.3 | | 295.7 | 23 | 2734.5 | 1367.7 | (y23++) |
| 6 | 661.3 | (b6+) | 331.2 | 22 | 2620.4 | 1310.7 | (y22++) |
| 7 | 790.4 | (b7+) | 395.7 | 21 | 2549.4 | 1275.2 | (y21++) |

| | | | | | | | |
|---|---|---|---|---|---|---|---|
| 8 | 847.4 | 424.2 | (b8++) | 20 | 2420.3 | 1210.7 | (y20++) |
| 9 | 976.4 | 488.7 | (b9++) | 19 | 2363.3 | 1182.2 | (y19++) |
| 10 | 1073.5 | 537.2 | (b10++) | 18 | 2234.3 | 1117.6 | (y18++) |

| 11 | 1202.5 | 601.8 | | 17 | 2137.2 | 1069.1 |
|---|---|---|---|---|---|---|
| 12 | 1315.6 | 658.3 | (b12++) | 16 | 2008.2 | 1004.6 |

| 13 | 1428.7 | | 714.9 | 15 | 1895.1 | | 948.0 | (y15++) |
|---|---|---|---|---|---|---|---|---|
| 14 | 1559.7 | (b14+) | 780.4 | 14 | 1782.0 | | 891.5 | (y14++) |
| 15 | 1672.8 | (b15+) | 836.9 | 13 | 1651.0 | (y13+) | 826.0 | (y13++) |

| | | | | | | | |
|---|---|---|---|---|---|---|---|
| 16 | 1743.9 | (b16+) | 872.4 | 12 | 1537.9 | 769.4 | (y12++) |

| 17 | 1857.9 | 929.5 | 11 | 1466.8 | | 733.9 | (y11++) |
|---|---|---|---|---|---|---|---|
| 18 | 2131.1 | 1066.0 | 10 | 1352.8 | | 676.9 | (y10++) |
| 19 | 2287.2 | 1144.1 | 9 | 1079.6 | | 540.3 | (y9++) |
| 20 | 2384.2 | 1192.6 | 8 | 923.5 | (y8+) | 462.3 | |
| 21 | 2455.3 | 1228.1 | 7 | 826.5 | (y7+) | 413.7 | |
| 22 | 2583.3 | 1292.2 | 6 | 755.5 | (y6+) | 378.2 | |
| 23 | 2680.4 | 1340.7 | 5 | 627.4 | (y5+) | 314.2 | (y5++) |

| | | | | | | |
|---|---|---|---|---|---|---|
| 24 | 2793.4 | 1397.2 | 4 | 530.3 | (y4+) | 265.7 |
| 25 | 2921.5 | 1461.3 | 3 | 417.3 | (y+) | 209.1 |

| | | | | | |
|---|---|---|---|---|---|
| 26 | 3035.6 | 1518.3 | 2 | 289.2 | 145.1 |
| 27 | — | — | 1 | 175.1 | 88.1 |

β₂-Microglobulin-2a Conjugate. Tandem MS data was obtained after analysis of the Protocol A digestion (modified to use 1% NH₄HCO₃ as digestion buffer instead of Tris buffer) by LC/MS by selecting m/z=1047.6 for collision-induced dissociation (Collision energy=35 V). Mass spectrum peaks (b2+), (b3+), (b11+), (b12+), (b13+), (b14+), (b19++), (b21++), (y2+), (y5+), (y12+), (y15+), (y7++), (y10++), (y11++), (y12++), (y13++), (y14++), (y15++), (y16++), (y17++), (y18++), (y19++), (y20++), and (y21++) confirmed the tagged/modified tryptophan product and are shown in Table D9. “AA #” refers to the amino acid number.

TABLE D9

| AA # | b(+) | b(++) | AA # | y(+) | | y(++) | |
|---|---|---|---|---|---|---|---|
| 1 | 116.0 | 58.5 | 23 | — | | — | |
| 2 | 389.1 | 195.1 | 22 | 3023.3 | | 1512.2 | |
| 3 | 476.2 | 238.6 | 21 | 2750.2 | | 1375.6 | (y21++) |
| 4 | 623.2 | 312.1 | 20 | 2663.2 | | 1332.1 | (y20++) |
| 5 | 786.3 | 393.7 | 19 | 2516.1 | | 1258.6 | (y19++) |
| 6 | 899.4 | 450.2 | 18 | 2353.1 | | 1177.0 | (y18++) |
| 7 | 1012.5 | 506.7 | 17 | 2240 | | 1120.5 | (y17++) |
| 8 | 1175.5 | 588.3 | 16 | 2126.9 | | 1064 | (y16++) |
| 9 | 1338.6 | 669.8 | 15 | 1963.8 | (y15+) | 982.4 | (y15++) |
| 10 | 1439.6 | 720.3 | 14 | 1800.8 | | 900.9 | (y14++) |
| 11 | 1568.7 (b11+) | 784.8 | 13 | 1699.7 | | 850.4 | (y13++) |
| 12 | 1715.8 (b12+) | 858.4 | 12 | 1570.7 | (y12+) | 785.8 | (y12++) |

| 13 | 1816.8 (b13+) | 908.9 | 11 | 1423.6 | 712.4 | (y11++) |
|---|---|---|---|---|---|---|
| 14 | 1913.9 (b14+) | 957.4 | 10 | 1322.6 | 661.9 | (y10++) |
| 15 | 2014.9 | 1008 | 9 | 1225.5 | 613.3 | |
| 16 | 2143.9 | 1072.5 | 8 | 1124.5 | 562.7 | |
| 17 | 2272.0 | 1136.5 | 7 | 995.4 | 498.2 | (y7++) |

| 18 | 2387.0 | 1194.0 | 6 | 867.3 | | 434.2 |
|---|---|---|---|---|---|---|
| 19 | 2516.1 | 1258.6 (b19++) | 5 | 752.4 | (y5+) | 376.7 |
| 20 | 2679.2 | 1340.1 | 4 | 623.3 | | 312.7 |
| 21 | 2750.2 | 1375.6 (b21++) | 3 | 460.2 | | 230.6 |
| 22 | 2964.2 | 1482.6 | 2 | 389.2 | (y2+) | 195.1 |

| | | | | | |
|---|---|---|---|---|---|
| 23 | — | — | 1 | 175.1 | 88.1 |

### F. Chemoproteomic Profiling

Cell culture and in-situ tryptophan probe treatment: Cell lines were cultured at 37° C. with 5% C₀2. Human embryonic kidney 293 cells expressing a mutant version of the SV40 large T antigen (HEKT293T cells) were grown in Dulbecco's Modified Eagle Medium (DMEM, Gibco) supplemented with 10% fetal bovine serum and 1% penicillin/streptomycin. For in-situ labeling, cells were grown to 90% confluence, washed with Dulbecco's phosphate buffered saline (DPBS), treated with 100x stocks of the tryptophan probes (the pyridinium salts) in OptiMem (GibCo) to generate final concentrations (0 μM, 1 μM, 10 μM, and 100 μM) in 1% dimethylsulfoxide (DMSO). Cells were incubated for 1 hour at 37° C. with 5% C₀2 followed by photo-activation at the respective wavelengths for 20 minutes at 4° C. Cells were harvested with a cell scraper, washed 3× with DPBS, and pellets were stored at −80° C. until further use. The following light sources were used for photo-activation:
Pyridinium salt 1a (also called, probe 1a): wavelength=302 nm; light source=Analytikjena, UVP 3UV Lamp, 8W.
Pyridinium salt 1b (also called, probe 1b): wavelength=320 nm; light source=Analytikjena, UVP 3UV Lamp, 8W, equipped with 320 nm long-pass filter.
Pyridinium salt 2b (also called, probe 2b): wavelength=440 nm; light source=Kessil PR-160L-440 nm LED, 45W.
Pyridinium salt 2d (also called, probe 2d): wavelength=440 nm; light source=Kessil PR-160L-440 nm LED, 45W.

In-situ tryptophan probe sample processing for peptide-level characterization: Cell pellets were lysed via sonication (QSonica) at 50% amplitude, 2 seconds ON, 3 seconds OFF, 30 seconds in 40 mM HEPES pH 7.5 (4-(2-hydroxyethyl)-1-piperazineethanesulfonic acid), 1.5 mM magnesium chloride (MgCl₂), 1% Sodium dodecyl sulfate (SDS), 0.8% NP-40 (also known as nonyl phenoxypolyethoxylethanol), and 1x protease inhibitors (Thermo). Protein concentration was determined by BCA (Pierce). 400 μg protein lysate were taken for copper-catalyzed azide-alkyne cycloaddition (CuAAC) reactions with 100 μM of the acid cleavable DAPBS Biotin Alkyne (Click Chemistry Tools), 250 μM copper sulfate (CuSO₄), 500 μM BTTP (C₂₀H₃₄N₁₀O₂) (Click Chemistry Tools), and 2.5 mM ascorbate and incubated for 60 minutes at R.T. Samples were precipitated with methanol:chloroform and re-suspended in 50 mM HEPES pH 8.0 containing 1% RapiGest SF (Waters). The samples were reduced with 10 mM dithiothreitol (DTT) (Pierce) and alkylated with 25 mM iodoacetamide (Pierce). The samples were diluted down to 0.1% RapiGest SF and treated with 4 μg Trypsin/LysC (Promega) at 37° C. for 16 hours. The digested peptides were than labeled with isobaric tandem mass tag (TMT) reagents (Thermo) as specified by the manufacturer. The labeled peptides were then combined, concentrated to remove acetonitrile, and re-suspended in 50 mM HEPES pH 8.0 containing 0.2% SDS. The labeled peptides were then treated with 100 μL Neutravidin resin as a 50% slurry (Peirce) for 16 hours at R.T. The following day, the Neutravidin resin was washed extensively with 50 mM HEPES pH 8.0 containing 0.2% SDS (3×), 50 mM HEPES pH 8.0 (5x), and double-distilled water (ddH₂O) (5×). The bound peptides were then cleaved from the resin by incubating with 100 μL 10% formic acid for 30 minutes (2×).

In lysate tryptophan probe treatment for protein-level characterization: HEK293T cell pellets were suspended in native lysis buffer (50 mM HEPES pH 7.5, 150 mM sodium chloride (NaCl), 1.5 mM MgCl₂, 1 mM DTT, 0.8% NP-40, 5% glycerol, and 1x protease inhibitors (Thermo)) at 2x the pellet volume. The samples were sonicated on ice for 30 seconds at 20% amplitude with a 2 second pulse ON and 3 second pulse OFF. Cell debris was removed by centrifugation at 20,000×g for 20 minutes at 4° C. Protein concentration was determined by bicinchoninic acid (BCA) (Pierce) and diluted down to 5 mg/mL with ChemProt buffer (50 mM HEPES pH 7.5, 150 mM NaCl, 1.5 mM MgCl₂, 1 mM DTT, 0.4% NP-40, and 1x protease inhibitors (Thermo). 5 mg cell lysates were treated with 100x stocks of the tryptophan probes (pyridinium salts 1a, 1b, and 2b) to generate final concentrations (0, 1, 10, and 100 μM) in 1% DMSO. Lysates were incubated for 1 hour at 4° C. followed by photo-activation at respective wavelengths for 20 minutes at 4° C. Excess probe was removed using Sephadex G-25 PD-10 desalting columns (GE Healthcare) pre-equilibrated with 50 mM HEPES pH 7.5, 150 mM NaCl, 1.5 mM MgCl₂, and 0.4% NP-40. The samples were then treated with 100 μL Neutravidin resin as a 50% slurry (Peirce). The samples were incubated overnight on a rotating apparatus at R.T. The following day, the beads were transferred to a MobiCol column (Boca Scientific), washed 3× with phosphate buffered saline (PBS) containing 0.2% SDS, washed 3x with PBS, and the enriched proteins were eluted with 2×NuPAGE lithium dodecyl sulfate (LDS) containing 10 mM DTT (Pierce) and heated for 30 minutes at 55° C. The eluent was collected and subsequently treated with 20 mM iodoacetamide (Pierce) for 30 minutes at R.T. protected from light. Samples were passed through detergent removal columns (Pierce) that were pre-equilibrated with 50 mM HEPES pH 8.0. The samples were treated with 2.5 μg Trypsin/LysC (Promega) at 37° C. for 16 hours. The following day, the sample was collected and prepped for isobaric labeling with TMT reagents (Thermo) as specified by the manufacturer.

Quantitative proteomics by two-dimensional nano liquid chromatography-tandem mass spectrometry (nLC-MS/MS): For protein-level characterization, the TMT-labeled sample were reconstituted in 0.1% formic acid (FA)/2% acetonitrile for offline high-pH reversed phase separation (RP10) using a Dionex UltiMate 3000 high-performance liquid chromatography (HPLC) system with fraction collection using Chromeleon v.6.8 (Thermo) software. Separation was achieved on a Waters Xbridge C18 3.5 μm 2.1×150 mm HPLC column (Waters) using a flow rate of 250 μL min-1 and the following ternary gradient and conditions: mobile phase A=water (HPLC grade), mobile phase B=acetonitrile (HPLC grade), and mobile phase C=200 mM ammonium acetate, pH 10. Mobile phase C is held at 10% throughout gradient. Starting conditions are 89% mobile phase A and 1% mobile phase B, ramping mobile phase B to 80% over 65 min. Fractions were collected in 96-well plates at 100 see per well. Fractions were reduced down to 12 fractions, dried, and reconstituted in 0.1% FA/2% acetonitrile for nLC-MS/MS. All data were acquired using a Orbitrap Fusion Lumos coupled to an Easy-nLC 1200 nanoflow liquid chromatograph operating at 300 nL min⁻¹. Peptides were cleaned up using a 1 cm online custom trap then eluted on a custom 20 cm laser pulled 75 μm column with C18 resin. Peptides were quantified using the synchronous precursor selection (SPS-MS3) or HCD method for TMT quantitation with 120k MS1 resolving power, 50 ms max injection time and 100% AGC. MS2 spectra were selected using the top ten most abundant features with a charge state between 2-6 using CID (collision induced dissociation) in the ion trap. The AGC was set to 100% with an isolation width of 0.7. MS3 was performed on the top 10 most abundant MS2 features between 400-1600 amu with a collision energy of 55%, AGC of 250%, and 50k resolution. For HCD TMT quantitation, the MS2 isolation width was set to 1.2 with a collision energy of 55%, AGC of 250%, and a resolution of 50K.

Mass spectrometry data processing: Peptide and protein identification and quantification were performed using Proteome Discoverer version 2.4 (Thermo). MS data were searched using the Mascot (Matrix Science) search engine against the UniProt reference database (human proteins, 42,233 entries, downloaded 2017) containing common contaminants and reversed sequences. Carbamidomethylated cysteine, oxidation of methionine, deamidation (NQ), and TMT modification on N-termini and lysine were set as dynamic modifications. Trypsin was specified as the proteolytic enzyme with up to one missed cleavage site allowed. Precursor and fragment ion tolerances were set to 10 ppm and 0.8 Da, respectively. Search results were filtered for a minimum of 2 unique peptides, 1% false discovery rate (FDR) peptide and protein identification and quantification, and common contaminants. Protein abundances were normalized and competition ratios were calculated using scripts provided by Proteome Discoverer. Dose-response curves were fitted to a three-parameter log logistic regression using GraphPad Prism. To map modified tryptophan, the peptide-level enriched datasets were searched against the mass corresponding to modified tryptophan harboring the cleaved tag (+282.169191 Da).

In-situ tryptophan probe sample processing for protein-level identification: Cell pellets were lysed via sonication (QSonica) at 50% amplitude, 2 seconds ON, 3 seconds OFF, 30 seconds in 50 mM HEPES pH 7.5, 1.5 mM MgCl₂, 1% SDS, 0.8% NP-40, and 1x protease inhibitors (Thermo). Protein concentration was determined by BCA (Pierce). 400 μg protein lysate was used for subsequent enrichment of probe-modified proteome. For the tryptophan-azide probe (Pyridinium salt 2d) modified proteome, CuAAC reactions were performed using 100 μM of the acid cleavable DADPS Biotin Alkyne (Click Chemistry Tools), 250 μM CuSO₄, 500 μM BTTP (Click Chemistry Tools), and 2.5 mM ascorbate and incubated for 60 minutes at R.T. Samples were precipitated with methanol:chloroform and re-suspended in 1% SDS/50 mM HEPES pH 8.0. The samples were diluted down to 0.2% SDS and added to 100 μL Neutravidin resin as a 50% slurry (Peirce). The samples were incubated overnight on a rotating apparatus. The following day, the beads were transferred to a MobiCol column (Boca Scientific), washed 3× with 50 mM HEPES pH 8.0 containing 0.2% SDS, washed 3× with 50 mM HEPES pH 8.0, and remaining proteins on beads were eluted off beads by treating resin with NuPAGE LDS sample buffer (2×) containing 10 mM DTT and heating at 55° C. for 30 minutes. Samples were then subjected to Western blot analysis as described below.

Western bolt analysis: Standard techniques were used for immunoblotting. Proteins were separated on 4-12% Bis-Tris NuPAGE gels (Invitrogen), transferred onto Trans-Blot Turbo Mini 0.2 μm nitrocellulose membranes (BioRad), and incubated with the indicated antibodies for immunoblotting. Antibodies used in this study were: horeseradish peroxidase (HRP)-linked anti-biotin (Cell Signaling Technology, #7075) Poly [ADP-ribose] polymerase 1 (PARP1) (Cell Signaling Technology, #9542) and nucleophosmin 1 (NPM1) (Cell Signaling Technology, #3542).

Solvent accessibility: The degree of solvent exposure of tryptophans in the dataset was calculated over residues resolved in solved structures by querying the PDBe-KB graph database for these values as calculated by the POPSCOMP method. In cases where multiple structures containing a tryptophan residue were available for a protein, the structure with the construct covering the largest fraction of the native protein was chosen. The unlabeled set comprised all unlabeled tryptophans in any protein that had one or more labeled tryptophans.

NPM1 Construct design: NPM1 residues 240-294 corresponding to the C-terminal DNA binding domain were fused to a combined 6×His-maltose binding protein (MBP) solubility tag on the N-terminus. An HRV 3C protease cleavage sequence (LEVLFQ/GP) was placed between the tags and NPM1 sequence and upon cleavage the remaining GP sequence corresponds to the NPM1 240-241 residues resulting in a natural peptide sequence without additional residues.

HIS-MBP-MP1[240-294] expression and purification: The tagged construct was placed under control of a T7/lac promoter and expressed in NiCo21(DE3) E. coli cells (New England Biolabs). Cells in one liter of Terrific Broth culture media were induced with 1 mM IPTG and incubated for an additional 4-5 hours at 30° C. before recovery. Cell pellets were re-suspended in lysis buffer (50 mM Tris-Cl, pH 7.5, 150 mM NaCl, 1 mM TCEP, 20 mM imidazole and protease inhibitor). Cells were lysed using the LM20 microfluidizer (Microfluidics) and tagged proteins were purified using an AKTA Pure FPLC and columns from Cytiva. Tagged NPM1[240-294] were isolated first using a 5 mL HisTrap FF crude column equilibrated with buffer (50 mM Tris-Cl, pH 7.5, 150 mM NaCl, 1 mM TCEP, 20 mM imidazole). After application the protein was washed with buffer containing 50 mM imidazole and finally eluted in buffer with 300 mM imidazole. Combined fractions containing the tagged NPM1 constructs were buffer exchanged into a lower salt buffer (50 mM Tris-Cl, pH 7.5, 100 mM NaCl, 1 mM TCEP). NPM1 protein was then run over a 5 mL HiTrap Q HP column to remove other protein and DNA impurities. The NPMI construct was found in the sample application fractions with very high yields of protein recovered (>100 mg estimated).

NPM1[240-294] peptide production: The recovered His-MBP-NPM1[240-294] protein was cleaved overnight at 4° C. with a His-tagged HRV 3C protease (Pierce™ HRV 3C Protease Solution Kit). The His-MBP tag and protease were removed by diluting cleaved samples 1:1 into a higher salt buffer (50 mM Tris-Cl, pH 7.5, 500 mM NaCl, 1 mM TCEP, 20 mM Imidazole) before passing over the 5 mL HiTrap FF crude column. The peptides were then further concentrated in a 3 kD MWCO centrifugal filter unit. Small amounts of higher molecular weight bands were found in a SDS-Page gel corresponding to residual tags or uncleaved product so peptides were further polished by passing through a 30 kDa MWCO centrifugal filter unit to remove the higher molecular weight products. Peptides were stored at −80° C. after a final buffer exchange step into storage buffer (50 mM Tris-Cl, 150 mM NaCl, 1 mM TCEP, 10% glycerol). Over 14 mg of peptide were recovered with high purity as confirmed on an SDS-page gel using a 16.5% Tris-Tricine gel (Bio-Rad). FIG. 11 shows an image of an SDS-Page gel of the purified NPM1[240-294] peptide.

In vitro NPM1 C-terminal domain labeling and characterization: Labeling of recombinant NPM1 with the pyridinium salt 2b was performed with the following conditions: 10 μM NPM1 was treated with DMSO or 100 μM pyridinium salt 2b (2% DMSO) in 50 mM HEPES pH 7.5, 150 mM NaCl, 1.5 mM MgCl₂, and 100 μM glutathione (GSH). Samples were irradiated at 450 nm (Kessil PR160L-440) at 100% intensity for 15 minutes at room temperature or 50° C. The reaction mixture was quenched with the addition of 4X LDS buffer (Invitrogen) containing 10 mM DTT (Pierce) and boiled for 10 minutes before resolving proteins on 4-12% Bis-Tris NuPAGE gels (Invitrogen), transferred onto a Trans-Blot Turbo Mini 0.2 μm nitrocellulose membrane (BioRad), blocked with 5% BSA in tris-buffered saline with Tween-20 (TBST), and incubated with HRP-linked anti-biotin (Cell Signaling Technology, #7075).

MS-MS data was collected for NPM1 Trp290 modification, PARP1 Trp79 modification, and PARP1 Trp318 modification. Tables F1, F2, and F3 show the MS-MS data collected for the NPM1 Trp290 modification, PARP1 Trp79 modification, and PARP1 Trp318 modification, respectively. The MS-MS data confirmed the tagged/modified tryptophan products. In Tables F1-F3, “AA #” refers to the amino acid number.

TABLE F1

| AA # | b(+) | b(++) | b(+++) | AA # | y(+) | y(++) | y(+++) |
|---|---|---|---|---|---|---|---|
| 1 | 377.20561 | 189.10644 | 126.40672 | 14 | — | — | — |
| 2 | 478.25329 | 239.63028 | 160.08928 | 13 | 1970.97190 | 985.98959 | 657.66215 |
| 3 | 593.28023 | 297.14375 | 198.43159 | 12 | 1869.92422 | 935.46575 | 623.97959 |
| 4 | 721.33881 | 361.17304 | 241.11779 | 11 | 1754.89728 | 877.95228 | 585.63728 |
| 5 | 850.38140 | 425.69434 | 284.13198 | 10 | 1626.83870 | 813.92299 | 542.95108 |
| 6 | 921.41851 | 461.21290 | 307.81102 | 9 | 1497.79611 | 749.40169 | 499.93689 |
| 7 | 1034.50258 | 517.75493 | 345.50571 | 8 | 1426.75899 | 713.88314 | 476.25785 |
| 8 | 1162.56116 | 581.78422 | 388.19190 | 7 | 1313.67493 | 657.34110 | 438.56316 |
| 9 | 1277.58810 | 639.29769 | 426.53422 | 6 | 1185.61635 | 593.31182 | 395.87697 |
| 10 | 1390.67216 | 695.83972 | 464.22891 | 5 | 1070.58941 | 535.79834 | 357.53465 |
| 11 | 1576.75147 | 788.87938 | 526.25534 | 4 | 957.50535 | 479.25631 | 319.83997 |
| 12 | 1704.81005 | 852.90866 | 568.94154 | 3 | 771.42603 | 386.21666 | 257.81353 |
| 13 | 2173.05856 | 1087.03292 | 725.02437 | 2 | 643.36746 | 322.18737 | 215.12734 |
| 14 | — | — | — | 1 | 175.11895 | 88.06311 | 59.04450 |

TABLE F2

| AA # | b(+) | b(++) | b(+++) | AA # | y(+) | y(++) | y(+++) |
|---|---|---|---|---|---|---|---|
| 1 | 367.22912 | 184.11820 | 123.08122 | 19 | — | — | — |
| 2 | 464.28188 | 232.64458 | 155.43215 | 18 | 2674.33103 | 1337.66915 | 892.11519 |
| 3 | 579.30883 | 290.15805 | 193.77446 | 17 | 2577.27826 | 1289.14277 | 859.76427 |
| 4 | 678.37724 | 339.69226 | 226.79726 | 16 | 2462.25132 | 1231.62930 | 821.42196 |
| 5 | 807.41983 | 404.21356 | 269.81146 | 15 | 2363.18290 | 1182.09509 | 788.39915 |
| 6 | 906.48825 | 453.74776 | 302.83427 | 14 | 2234.14031 | 1117.57379 | 745.38495 |
| 7 | 1021.51519 | 511.26123 | 341.17658 | 13 | 2135.07190 | 1068.03959 | 712.36215 |
| 8 | 1078.53665 | 539.77197 | 360.18374 | 12 | 2020.04495 | 1010.52612 | 674.01984 |
| 9 | 1225.60507 | 613.30617 | 409.20654 | 11 | 1963.02349 | 982.01538 | 655.01268 |
| 10 | 1312.63710 | 656.82219 | 438.21722 | 10 | 1815.95508 | 908.48118 | 605.98988 |
| 11 | 1441.67969 | 721.34348 | 481.23141 | 9 | 1728.92305 | 864.96516 | 576.97920 |
| 12 | 1554.76375 | 777.88552 | 518.92610 | 8 | 1599.88046 | 800.44387 | 533.96500 |
| 13 | 1710.86486 | 855.93607 | 570.95981 | 7 | 1486.79639 | 743.90183 | 496.27032 |
| 14 | 2179.11337 | 1090.06032 | 727.04264 | 6 | 1330.69528 | 665.85128 | 444.23661 |
| 15 | 2294.14031 | 1147.57379 | 765.38495 | 5 | 862.44678 | 431.72703 | 288.15378 |
| 16 | 2409.16725 | 1205.08727 | 803.72727 | 4 | 747.41983 | 374.21356 | 249.81146 |
| 17 | 2537.22583 | 1269.11655 | 846.41346 | 3 | 632.39289 | 316.70008 | 211.46915 |
| 18 | 2665.28441 | 1333.14584 | 889.09965 | 2 | 504.33431 | 252.67080 | 168.78296 |
| 19 | — | — | — | 1 | 376.27574 | 188.64151 | 126.09676 |

TABLE F3

| AA # | b(+) | b(++) | b(+++) | AA # | y(+) | y(++) | y(+++) |
|---|---|---|---|---|---|---|---|
| 1 | 317.20224 | 159.10476 | 106.40560 | 15 | — | — | — |
| 2 | 432.22918 | 216.61823 | 144.74791 | 14 | 2162.04257 | 1081.52492 | 721.35237 |
| 3 | 503.26629 | 252.13679 | 168.42695 | 13 | 2047.01563 | 1024.01145 | 683.01006 |
| 4 | 666.32962 | 333.66845 | 222.78139 | 12 | 1975.97852 | 988.49290 | 659.33102 |
| 5 | 829.39295 | 415.20011 | 277.13583 | 11 | 1812.91519 | 906.96123 | 604.97658 |
| 6 | 989.42360 | 495.21544 | 330.47938 | 10 | 1649.85186 | 825.42957 | 550.62214 |
| 7 | 1090.47128 | 545.73928 | 364.16194 | 9 | 1489.82121 | 745.41424 | 497.27859 |
| 8 | 1147.49274 | 574.25001 | 383.16910 | 8 | 1388.77353 | 694.89040 | 463.59603 |
| 9 | 1262.51968 | 631.76348 | 421.51141 | 7 | 1331.75207 | 666.37967 | 444.58887 |
| 10 | 1361.58810 | 681.29769 | 454.53422 | 6 | 1216.72512 | 608.86620 | 406.24656 |
| 11 | 1462.63578 | 731.82153 | 488.21678 | 5 | 1117.65671 | 559.33199 | 373.22375 |
| 12 | 1533.67289 | 767.34008 | 511.89581 | 4 | 1016.60903 | 508.80815 | 339.54120 |
| 13 | 2001.92139 | 1001.46434 | 667.97865 | 3 | 945.57192 | 473.28960 | 315.86216 |
| 14 | 2102.96907 | 1051.98817 | 701.66121 | 2 | 477.32341 | 239.16535 | 159.77932 |
| 15 | — | — | — | 1 | 376.27574 | 188.64151 | 126.09676 |

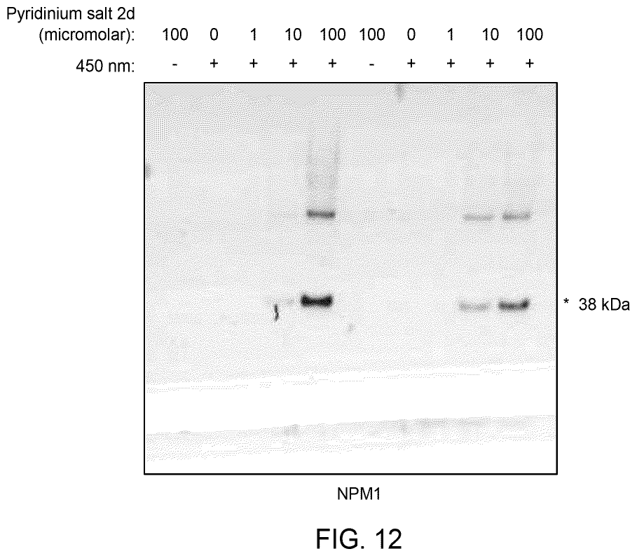

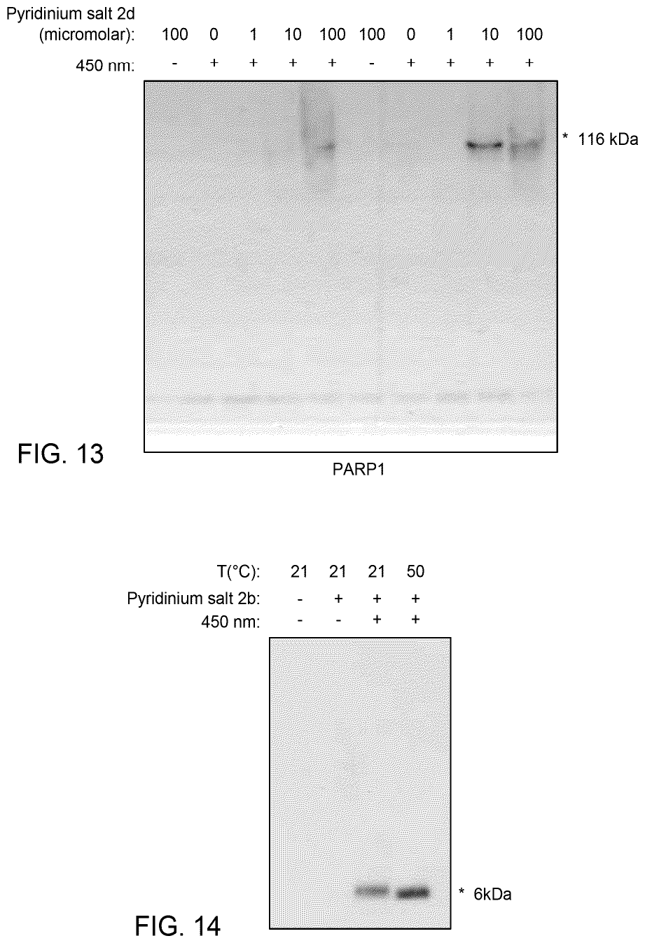

FIG. 12 shows an image of a Western blot validating the presence of NPM1 in enriched proteins from in-situ chemoproteomic profiling of HEK293T cells using example pyridinium salt 2d. FIG. 13 shows an image of a Western blot validating the presence of PARP1 in enriched proteins from in-situ chemoproteomic profiling of HEK293T cells using example pyridinium salt 2d. The identification of NPM1 and PARP1 as enriched targets with pyridinium salt 2d was validated as shown in FIG. 12 and FIG. 13. The HEK293T cells were treated with varying concentrations of pyridinium salt 2d both in the presence and absence of blue light followed by protein-level enrichment. The resulting enriched protein mixtures were then analyzed via Western analysis and found to contain both proteins when cultures were irradiated in the presence of pyridinium salt 2d. No enrichment of these proteins was found in either the absence of pyridinium salt or the absence of light.

FIG. 14 shows an image of a Western blot of the labeling of recombinant NPM1 C-terminal domain using example pyridinium salt 2b. The recombinant NPM1 C-terminal domain is a construct (residues 240-294). This NPM1 (240-294) construct was irradiated for about 15 minutes at about 440 nm with pyridinium salt 2b, about 50 mM pH 7.5 HEPES, 150 mM MgCl₂, and 100 μM glutathione. Western analysis, as shown in FIG. 14, confirmed photo-dependent labeling of the construct. Temperature dependence of the labeling was also observed, wherein labeling at about 50° C. leads to a higher level of labeling.

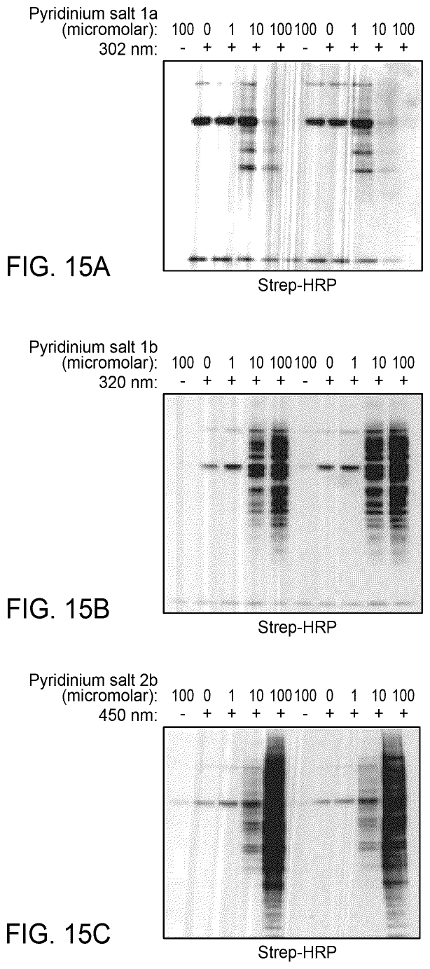

FIGS. 15A-15C show Western blots of elution profiles of chemoproteomic profiling of HEKT293T lysates with pyridinium salt 1a (FIG. 15A), pyridinium salt 1b (FIG. 15B), and pyridinium salt 2b (FIG. 15C). The western blots used streptavidin-HRP of elution profiles for the pyridinium salts 1a, 1b, and 2b) before digestion. The results indicated that each probe displays concentration and optically dependent proteome labeling profiles.

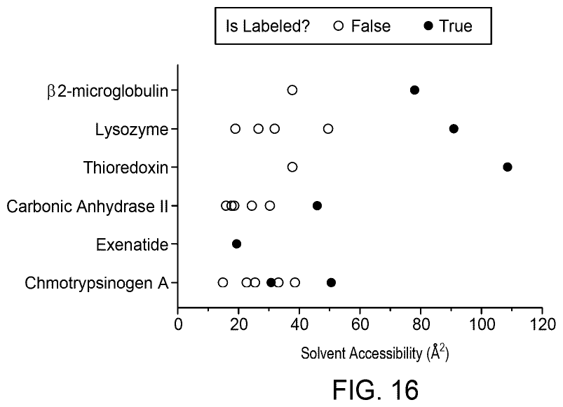

FIG. 16 is a scatter plot of the solvent accessibility of all tryptophan residues in the proteins investigated herein and whether the tryptophan residue was labeled under the conditions tested. The labeled tryptophan (Trp) residues for each substrate are shown in Table F4. Surface exposure data was not available for Octreotide and therefore not included.

TABLE F4

| Protein name | Modified Trp |
|---|---|
| leuprolide | Trp-03 |
| exenatide | Trp-025 |
| thioredoxin | Trp-31 |
| β₂-microglobulin | Trp-60 |
| lysozyme | Trp-62 |
| Chymotrypsinogen | Trp-215 |
| | Trp-237 |
| Carbonic anhydrase II | Trp-243 |

Labeled tryptophan residues for each substrate (NPM1 and PARP1) and the calculated solvent accessibility data are shown in Table F5.

TABLE F5

| Protein | Residue | Modified? | Solvent accessibility, Å² |
|---|---|---|---|
| NPM1 | Trp-288 | No | 49.3934 |
| | Trp-290 | Yes | 101.0552 |
| PARP1 | Trp-79 | Yes | 45.6739 |
| | Trp-246 | No | 36.1597 |
| | Trp-318 | Yes | 46.4489 |

### G. Non-Limiting Discussion

A group of tryptophan-containing peptides and proteins in which the tryptophan residues are located in differing environments were investigated for labeling (modifying) using example pyridinium salts. Smaller peptides, such as octreotide and leuprolide were each labeled (modified) efficiently under violet LED irradiation condition using pyridinium salt 2a as an example pyridinium salt. Exenatide, a 4.2 kDa polypeptide that possesses a single turn of the peptide backbone that partially obscures its single tryptophan residue, displayed slightly diminished, but still efficient, conversion (about 70%). Thioredoxin (Trx), an 11 kDa protein essential to disulfide formation, possesses two tryptophan residues, two tyrosine residues, and in its reduced form, two cysteine residues. Irradiation of Trx in the presence of example pyridinium salt 2a proceeded smoothly to afford tryptophan modification at Trp-31. During the course of the labeling process, disulfide bond formation was noted.

Labeling of β₂-microglobulin (B2M) was then investigated. B2M is an 11.8 kDa structural component of the class I major histocompatibility complex which includes two tryptophan residues, one that is fully solvent-exposed and one that is largely buried, as well as six surface exposed tyrosine residues. Irradiation of B2M with example pyridinium salt 2a for about 30 min led to almost exclusive mono-labeling at solvent-exposed Trp-60 in about 88% conversion.

Chymotrypsinogen A, a 25 kDa protein, possesses eight tryptophan residues and four tyrosine residues, is efficiently labeled in about 90% conversion, with a mono:di:tri labeling ratio of about 21:8:1. Digestion/MS-MS elucidated Trp-215 and Trp-237 to be the detectable locations of labeling in chymotrypsinogen A. Labeling of bovine carbonic anhydrase II (CAII) was then investigated. CAII possesses seven tryptophan residues, most of which are either completely buried or have very limited solvent exposure. As a result, CAII displayed much lower levels of conversion (about 34%), with Trp-243 being the identifiable site of modification. Taken together, these results establish the selectivity of example pyridinium salt 2a for tryptophan over an array of environments.

Plotting the solvent accessibility of each tryptophan residue in selected peptides and proteins weighing greater than 4 kDa and comparing against modification sites indicates that example pyridinium salt 2a typically selects for the most solvent-accessible residue when multiple choices are present (FIG. 16). In examples where there is a single tryptophan residue that is poorly exposed, such as exenatide, modification can still be observed, albeit with reduced conversion under the conditions investigated. The data may suggest that example pyridinium salt 2a may be sensitive to local steric environments, not just with respect to surface exposure but also to adequate surface exposure of the indolic C2 position as this position may be the preferred site of carbamylation.

It is also noted that the labeling reactions generally demonstrate clean reaction profiles, indicating that example pyridinium salt 2a does not cause significant levels of photodegradation.

Lysozyme was labeled with example pyridinium salts 2b, 2c, or 2d to investigate, the transferring group scope using the pyridinium scaffold. In that regard, it was found that affinity tags such as biotin (e.g., example pyridinium salt 2b) can be efficiently transferred to lysozyme in about 82% conversion. Protein labeling with example pyridinium salt 2b proceeded at a much faster rate than with example pyridinium salt 2a, with complete reactivity observed in less than about 10 min, as opposed to about 60 min with example pyridinium salt 2a. As described above, the fluorescence lifetime experiments show that example pyridinium salt 2b has a shorter 12 than example pyridinium salt 2a, while a comparison of emission spectra reveal that example pyridinium salt 2b has a higher fluorescence quantum yield than example pyridinium salt 2a, suggesting a correlation between fluorescence quantum yield and kinetics.

Desthiobiotinylation with example pyridinium salt 2c also proceeded with an enhanced rate but a larger excess of probe was used (concentration of example pyridinium salt 2c=about 500 μM versus concentration of example pyridinium salt 2b=about 200 M) in order to observe comparable labeling efficiency under the conditions tested. The difference in reactivity between these two structurally similar probes may, in part, be attributed to subtle differences in water solubility. The azide probe of example pyridinium salt 2d also showed enhance rates compared to example pyridinium salt 2a and was smoothly transferred to lysozyme in about 84% conversion with just about 20 min of irradiation time.

These results indicate that embodiments described herein show efficient reactivity and selectivity toward tryptophan modification in purified peptide/protein systems.

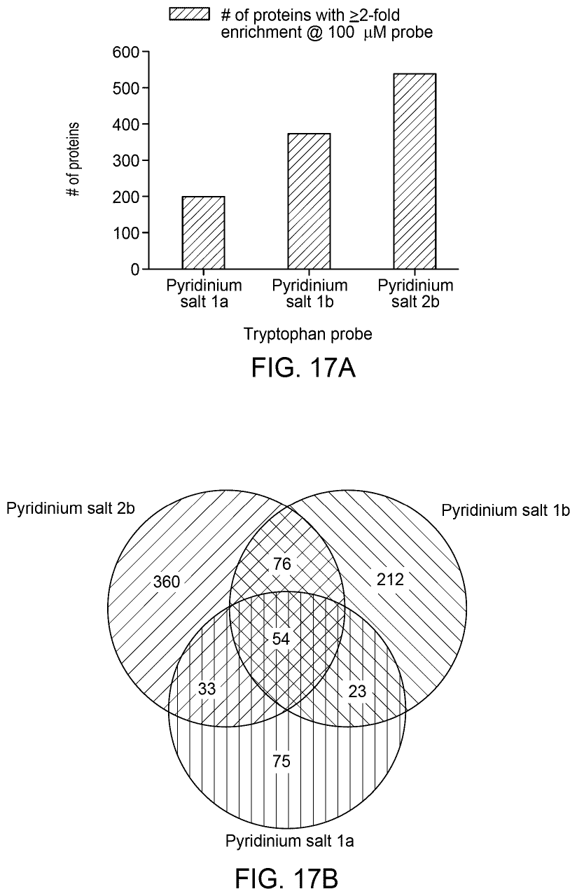

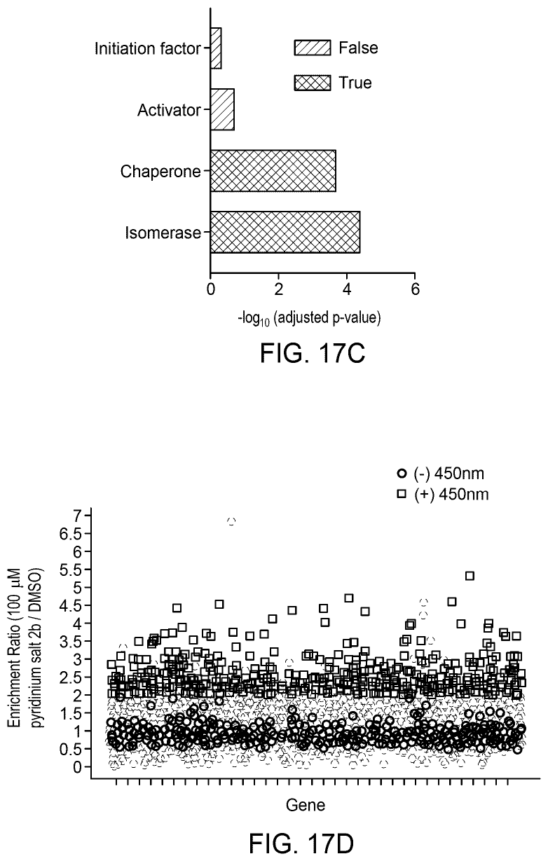

The chemoproteomic profiling capabilities of example pyridinium salts were then investigated. Three biotinylated tryptophan probes (example pyridinium salts 1a, 1b, and 2b) were investigated proteome profiling of HEK293T lysates. The lysates (about 5 mg/mL) were incubated with the desired probe at varying concentrations (about 0-100 μM) for about 60 min and subsequently irradiated for about 20 min with the following light sources: unfiltered 302 nm light for example pyridinium salt 1a, filtered 320 nm light for example pyridinium salt 1b, and 440 nm light for example pyridinium salt 2b. Biotinylated proteins were then enriched via neutravidin and washed to remove nonspecifically bound proteins. The enriched proteins were eluted and subsequently digested in solution followed by tandem mass tag (TMT) labeling and protein identification and quantitation by LC/MS-MS. Western blot analysis using streptavidin-HRP of elution profiles for the probes (example pyridinium salts 1a, 1b, and 2b) before digestion indicated that each probe displays concentration and optically dependent proteome labeling profiles. A loss of signal with example pyridinium salt 1a (the trimethyl pyridinium salt) at the highest assayed conditions, about 100 μM (FIGS. 15A-15C). It is possible that this loss of signal correlates to photodegradation associated with the generation of an excess of high energy photoexcited states of example pyridinium salt 1a, which could originate from the lower wavelength light emitted by the unfiltered 302 nm light source. Both example pyridinium salt 1b and example pyridinium salt 2b showed dose-response profiles. Following enrichment and digestion, LC/MS-MS analysis led to the identification (from ≥2 unique peptides) of 185 proteins with example pyridinium salt 1a, 365 proteins with example pyridinium salt 1b, and 523 proteins with example pyridinium salt 2b that showed ≥2-fold enrichment at about 100 μM concentration versus DMSO as shown in FIG. 17A. A comparison of proteome coverage of each probe reveals that example pyridinium salt 2b has the highest percentage (about 69%) of unique enriched proteins as shown in FIG. 17B.

In order to understand what types of protein functions are enriched with example pyridinium salt 2b, an enrichment analysis of proteins displaying ≥2-fold enrichment with example pyridinium salt 2b versus all proteins detected in the experiment was performed and is shown in FIG. 17C. This analysis indicated that chaperone and isomerase proteins have an especially high degree of enrichment against the background. Analysis of enrichment profiles for identified proteins with example pyridinium salt 2b in both the presence and absence of photoirradiation clearly illustrates both the photo-induced reactivity of this probe as well as its thermal inertness in the absence of light as shown in FIG. 17D.

Investigations into the labeling and identifying tryptophan modifications through intracellular photo-induced electron transfer (PET) labeling in cultured HEK293T cells were performed. The example pyridinium salt 2d (having a clickable azide functionality) was used for these investigations. Here, live HEK293T cell cultures were incubated with about 1-100 μM concentrations of example pyridinium salt 2d for about 60 min at about 37° C. followed by photoirradiation with about 440 nm light for about 20 min at about 4° C. Lysates were then generated followed by CuAAC to install an acid-cleavable alkyne-DADPS-biotin (DADPS is dialkoxy-diphenyl silane) affinity tag, digestion, TMT labeling, and recombination of TMT-labeled samples. Enrichment of this mixture with neutravidin followed by release of captured tryptic peptides with 10% formic acid yielded peptides harboring covalent modifications that were then identified and quantified by LC/MS-MS.

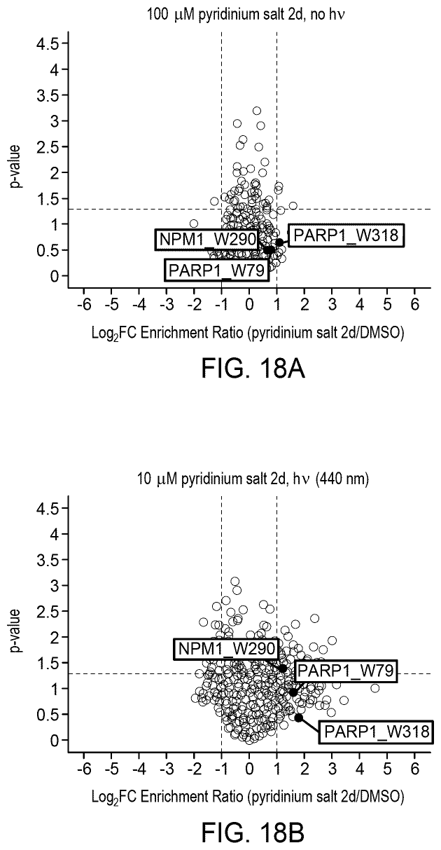

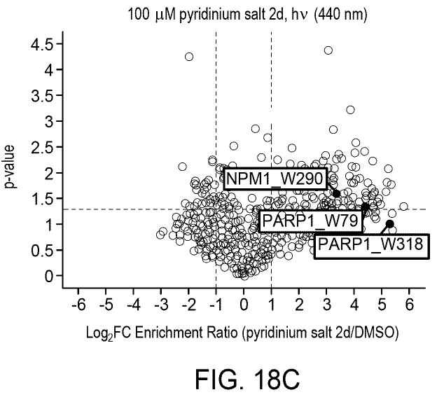

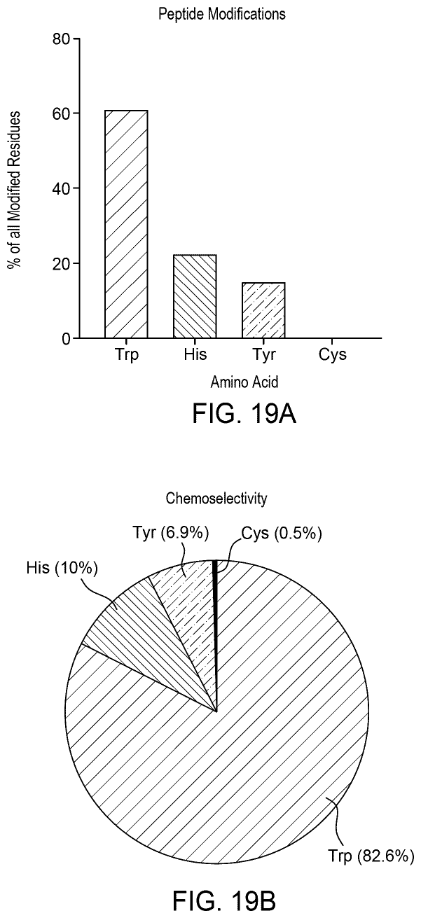

As evidenced in FIGS. 18A-18C, example pyridinium salt 2d can enable light- and dose-dependent enrichment of Trp-modified peptides, with measurable two-fold enrichment at low (about 10 μM) concentrations of probe. In FIGS. 18A-18C, the open circles indicate enriched peptides harboring tryptophan modifications and the filled circles indicate labeled tryptophan in NPM1 and PARP1. Example pyridinium salt 2d also displays robust thermal stability under whole cell culture conditions, and the lack of peptide enrichment without 440 nm photoirradiation demonstrates spatiotemporal control. About 100 μM of example pyridinium salt 2d enabled detection of 290 Trp-modified peptide fragments showing dose-response behavior that correspond to 209 unique proteins (blue dots). Selectivity for tryptophan modification is maintained in situ and demonstrates the ability to enrich the tryptophan-ome directly from whole cell culture. Analysis of the chemoselectivity profile of example pyridinium salt 2d revealed 221 unique peptides harboring a tryptophan (Trp) modification, with minor levels of modifications observed at histidine (His), tyrosine (Tyr), and cysteine (Cys) as shown in FIG. 19A. When the relative amino acid frequency of these residues in human proteins is taken into account, example pyridinium salt 2d displayed about 82% selectivity for tryptophan as shown in FIG. 19B.

Proteins with detected tryptophan modifications were analyzed against the UniProt database for subcellular localization profiles. A majority of identified proteins were found to have at least partial localization within the nucleus and the cytoplasm followed by the mitochondria and endoplasmic reticulum. In addition to evidencing that example pyridinium salt 2d readily crosses the plasma membrane, this analysis also indicates that the net positive charge of example pyridinium salt 2d does not restrict tryptophan modification to mitochondrial proteins. Rather, example pyridinium salt 2d is able to penetrate a wide variety of important subcellular regions and can enrich the tryptophan-ome in a wide array of areas.

Chemoproteomic profiling with example pyridinium salt 2d enabled dose-dependent enrichment of functionally critical Trp residues. For example, chemoproteomic tryptophan profiling with example pyridinium salt 2d enabled in-situ labeling of Trp290 of the nuclear protein NPM1. NPMI is a protein involved in a wide array of cellular functions revolving around genomic homeostasis. Trp288 has a lower calculated solvent accessibility score compared to Trp290 and was not labeled (see Table F5). Mutation of Trp290 and Trp288, which are commonly found in cases of acute myeloid leukemia, result in misfolding of the C-terminal domain and translocation of the protein to the cytosol. The modification and dose-dependent enrichment of Trp79 and Trp318 in PARP1 (an essential protein in DNA damage detection and repair, gene transcription regulation, and cell death signaling) was also observed. Trp79 and Trp318 participate in noncovalent interactions at the interface between various domains of PARP1, and mutation of these residues resulted in either partial (Trp79) or complete (Trp318) loss of catalytic activity.

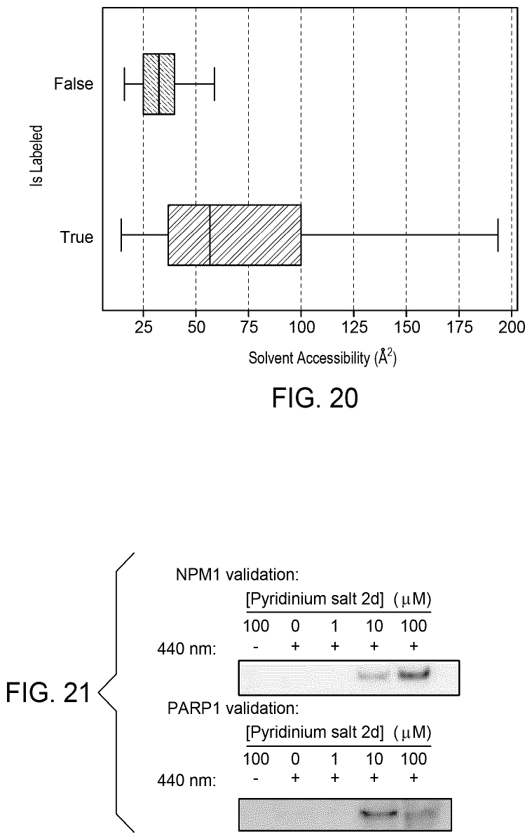

The data indicated that example pyridinium salts may select for tryptophan residues with the greatest solvent accessibility when multiple tryptophan residues are present. A survey of all tryptophan residues found on the proteins identified from the in situ studies (for which structural data is available) for solvent accessibility (FIG. 20) was then conducted. Given a maximum theoretical surface exposure of about 264 Å², modified tryptophan residues were found to be about 21% accessible (average accessible area of about 56 Å²), while unmodified tryptophan residues were only 12% accessible (average accessible area of about 32 Å²), a statistically significant difference. This data may further illustrate a preference for pyridinium salts to label tryptophan residues with enhanced surface exposure.

While the investigations showed the ability to enrich tryptophan residues with a known function, cross-referencing identified proteins against the Drugbank database revealed that a majority of identified proteins (about 75%) do not have Drugbank entries. A search of modified tryptophan residues for residue/region-specific annotation in the UniProt database indicated that a large majority (about 92%) of modified residues have no functional annotation assigned to them. The most common functional annotations found involved tryptophan residues located in trans-membrane regions of the parent protein (about 6%), while about 2% of tryptophan residues had an established function based upon mutagenesis experiments. This very low percentage of residue annotation contrasts to other chemoproteomic profiling workflows that target more highly studied residues and suggests potential for this method to enable the discovery of functionally important tryptophan residues.

The identification of NPM1 and PARP1 as enriched targets with example pyridinium salt 2d was validated as shown in FIG. 21. HEK293T cells were treated with varying concentrations of example pyridinium salt 2d both in the presence and absence of blue light followed by protein-level enrichment. The resulting enriched protein mixtures were then analyzed via Western analysis and found to contain both proteins when cultures were irradiated in the presence of example pyridinium salt 2d. No enrichment of these proteins was found in either the absence of pyridinium salt or the absence of light.

Enrichment of NPM1 was further validated by directly labeling an NPM1 C-terminal construct (residues 240-294) which possesses both residues in NPM1. Briefly, the NPM1 240-294 construct was irradiated at about 440 nm with example pyridinium salt 2b for about 15 minutes. The solution of example pyridinium salt 2b and NPM1 240-294 construct for this experiment also included about 50 mM pH 7.5 HEPES, about 150 mM MgCl₂, and about 100 μM glutathione. Western analysis, as shown in FIG. 14, confirmed the photo-dependent labeling of the construct. A temperature dependence of the labeling was also observed, wherein labeling at about 50° C. can lead to a higher level of labeling. Such results may indicate that the NPM1 240-294 construct is tightly folded at room temperature under the labeling conditions used, with elevated temperatures causing partial relaxation of the structure, in turn leading to enhanced labeling.

Embodiments of the present disclosure generally relate to pyridinium salts, compositions thereof, and to methods of use. Overall, pyridinium salts described herein can enable tryptophan modification in molecules such as polypeptides and proteins using light such as visible light and/or UV light. Even at low concentrations, pyridinium salts described herein can label tryptophan-containing polypeptides with good conversion and selectivity. The transferring group (the A group of formula (I)) can be used to install functional handles useful for, e.g., chemoproteomics and general bioconjugation strategies on to polypeptides and proteins. In addition, pyridinium salts described herein are useful for chemoproteomic profiling, enabling enrichment of the tryptophan-ome from both lysates and live cell culture.

### Embodiments Listing

The present disclosure provides, among others, the following embodiments, each of which can be considered as optionally including any alternate embodiments:

Clause A1. A composition, comprising: a pyridinium salt represented by formula (I), an ion thereof, or combinations thereof:

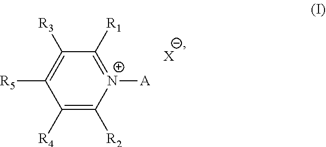

wherein: A of formula (I) is an unsubstituted C1 to C40 hydrocarbyl, substituted C1 to C40 hydrocarbyl, or a functional group comprising at least one element of Group 13-17 of the periodic table of the elements; each of R¹, R², R³, R⁴, and R⁵ of formula (I) is independently hydrogen, unsubstituted C1 to C40 hydrocarbyl, substituted C1 to C40 hydrocarbyl, or a functional group comprising at least one element of Group 13-17 of the periodic table of the elements; and X of formula (I) is a monoatomic or polyatomic anion.

Clause A2. The composition of Clause A1, wherein: A of formula (I) is an unsubstituted C1 to C20 hydrocarbyl or a substituted C1 to C20 hydrocarbyl, or a functional group comprising at least one element of Group 13-17 of the periodic table of the elements; and/or each of R¹, R², R³, R⁴, and R⁵ of formula (I) is independently hydrogen, unsubstituted C1 to C20 hydrocarbyl, substituted C1 to C20 hydrocarbyl, or a functional group comprising at least one element of Group 13-17 of the periodic table of the elements.

Clause A3. The composition of Clause A2, wherein A of formula (I) is

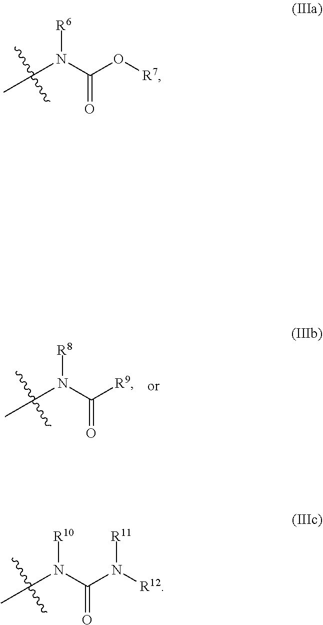

wherein: the wavy bond of formulas (IIIa)-(IIIc) represents the connection to the nitrogen atom of the pyridine ring; and each of R⁶, R⁷, R⁸, R⁹, R¹⁰, R¹¹, and R¹² of formulas (IIIa)-(IIIc) is independently hydrogen, unsubstituted C1 to C20 hydrocarbyl, substituted C1 to C20 hydrocarbyl, or a functional group comprising at least one element of Group 13-17 of the periodic table of the elements.

Clause A4. The composition of any one of Clauses A1-A3, wherein: each of R¹, R², R³, and R⁴ of formula (I) is independently hydrogen, a C1 to C10 alkyl, or a C1 to C10 alkoxy; and/or R⁵ is an unsubstituted aryl or a substituted aryl.

Clause A5. The composition of Clause A1, wherein: each of R¹ and R² of formula (I) is independently C1-C4 alkyl; each of R³ and R⁴ of formula (I) is hydrogen; and/or R⁵ of formula (I) is unsubstituted phenyl, substituted phenyl, unsubstituted naphthyl, or substituted naphthyl.

Clause A6. The composition of any one of Clauses A1-A5, wherein R⁵ of formula (I) is represented by formula (IIa):

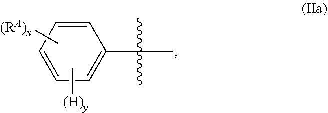

wherein: the wavy bond of formula (IIa) represents the connection to the pyridine ring; H of formula (IIa) is a hydrogen atom on the aromatic ring; R^{A} of formula (IIa) is an unsubstituted hydrocarbyl having from 1 to 20 carbon atoms, a substituted hydrocarbyl having from 1 to 20 carbon atoms; x of formula (IIa) is from 0 to 5, and when x is more than 1, each R^{A} group is the same or different; y of formula (IIa) is from 1 to 5; and x+y of formula (IIa) is 5.

Clause A7. The composition of Clause A6, wherein each R^{A} of formula (IIa) is independently an unsubstituted hydrocarbyl having from 1 to 4 carbon atoms or an alkoxy group having from 1 to 4 carbon atoms.

Clause A8. The composition of Clause A6 or Clause A7, wherein each R^{A} of formula (IIa) is independently methyl, ethyl, methoxy, or ethoxy.

Clause A9. The composition of any one of Clauses A1-A5, wherein R⁵ of formula (I) is

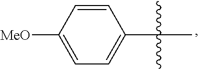 [SMILES: *c1ccc(OC)cc1]

the wavy bond representing the connection to the pyridine ring.

Clause A10. The composition of any one of Clauses A1-A5, wherein R⁵ of formula (I) is represented by formula (IIb):

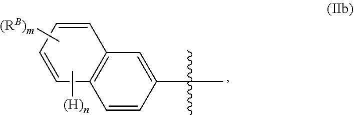

wherein: the wavy bond of formula (IIb) represents the connection to the pyridine ring; H of formula (IIb) is a hydrogen atom on the naphthyl ring; R^{B} of formula (IIb) is an unsubstituted hydrocarbyl having from 1 to 20 carbon atoms, a substituted hydrocarbyl having from 1 to 20 carbon atoms; m of formula (IIb) is from 0 to 7, and when m is more than 1, each R^{B} group is the same or different; n of formula (IIb) is from 0 to 7; and m+n of formula (IIb) is 7.

Clause A11. The composition of Clause A10, wherein each R^{B} of formula (IIb) is independently an unsubstituted hydrocarbyl having from 1 to 4 carbon atoms or an alkoxy group having from 1 to 4 carbon atoms.

Clause A12. The composition of Clause A10, wherein each R^{B} of formula (IIb) is independently methyl, ethyl, methoxy, or ethoxy.

Clause A13. The composition of any one of Clauses A1-A5, wherein R⁵ of formula (I) is

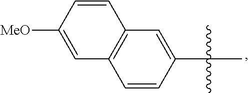 [SMILES: *c1ccc2cc(OC)ccc2c1]

the wavy bond representing the connection to the pyridine ring.

Clause A14. The composition of any one of Clauses A1-A13, wherein X⁻ of formula (I) is selected from the group consisting of F⁻, Cl⁻, Br⁻, and I⁻, BF₄⁻, CF₃CO₂⁻, BCl₄⁻, BBr₄⁻, BI₄⁻, NO₃⁻, NO₂⁻, ClO₄⁻, IO₃⁻, ClO₃⁻, BrO₃⁻, ClO₂⁻, OCl⁻, OBr⁻, CN⁻, OCN⁻, SCN⁻, KMnO₄⁻, HSO₄⁻, HSO₃⁻, RSO₃⁻, H₂PO₄⁻, OH⁻, CH₃CO₂⁻, HCO₂⁻, HCO₃⁻, H₃CC₆H₄SO₂⁻, F₃CSO₃⁻, CH₃SO₃⁻, C₇H₅O₂⁻, C₃H₅O₃⁻, (CH₃(CH₂)₃)₂HPO₄⁻, and (C₆H₅)₄B⁻.

Clause A15. The composition of any one of Clauses A1-A14, wherein X⁻ of formula (I) is selected from the group consisting of F⁻, Cl⁻, Br⁻, I⁻, BF₄⁻, CF₃CO₂⁻, PF₆⁻, H₃CC₆H₄SO₂⁻ (tosyl), SCN⁻, BCl₄⁻, BBr₄⁻, BI₄⁻, F₃CSO₃⁻, CH₃CO₂⁻, CH₃SO₃⁻, C₇H₅O₂⁻, C₃H₅O₃⁻, (CH₃(CH₂)₃)₂HPO₄⁻, and (C₆H₅)₄B⁻.

Clause B1. A method for modifying a tryptophan moiety, comprising:
forming a mixture comprising:
   a molecule containing a tryptophan moiety; and
   a composition comprising a pyridinium salt, an ion thereof, or a combination thereof; and
exposing the mixture to light to chemically modify the tryptophan moiety of the molecule.

Clause B2. The method of Clause B1, wherein the pyridinium salt is represented by formula (I):

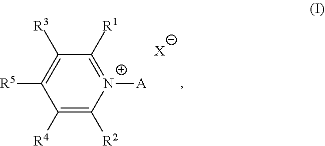

wherein: A of formula (I) is an unsubstituted C1 to C40 hydrocarbyl, substituted C1 to C40 hydrocarbyl, or a functional group comprising at least one element of Group 13-17 of the periodic table of the elements; each of R¹, R², R³, R⁴, and R⁵ formula (I) is independently hydrogen, unsubstituted C1 to C40 hydrocarbyl, substituted C1 to C40 hydrocarbyl, or a functional group comprising at least one element of Group 13-17 of the periodic table of the elements; and X⁻ is a monoatomic or polyatomic anion.

Clause B3. The method of Clause B1 or B2, wherein the modified tryptophan of the molecule is represented by formula (VI):

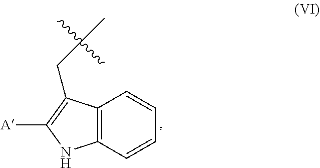

wherein: the wavy bond of formula (VI) represents a connection to the molecule; and A′ of formula (VI) is A of formula (I).

Clause B4. The method of any one of Clauses B1-B3, wherein: a wavelength or wavelength range of the light is from about 300 nm to about 440 nm; the mixture further comprises an antioxidant; the molecule is a biological molecule, a biologically-derived molecule, or a synthetic molecule; or combinations thereof.

Clause C1. A method of polypeptide modification, comprising: irradiating a mixture comprising a pyridinium salt and a polypeptide containing at least one tryptophan residue with ultraviolet light, visible light, or both to chemically modify the at least one tryptophan residue of the polypeptide, the pyridinium salt represented by formula (I):

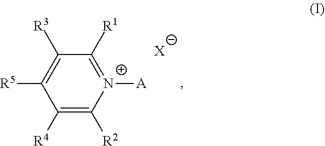

wherein: A of formula (I) is an unsubstituted C1 to C40 hydrocarbyl, substituted C1 to C40 hydrocarbyl, or a functional group comprising at least one element of Group 13-17 of the periodic table of the elements; each of R¹, R², R³, R⁴, and R⁵ formula (I) is independently hydrogen, unsubstituted C1 to C40 hydrocarbyl, substituted C1 to C40 hydrocarbyl, or a functional group comprising at least one element of Group 13-17 of the periodic table of the elements; and X⁻ is a monoatomic or polyatomic anion.

Clause D1. A composition, comprising: a pyridinium salt represented by formula (V), an ion thereof, or combinations thereof:

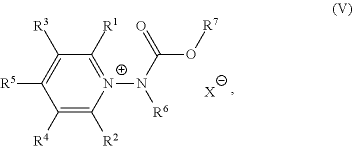

wherein: each of R¹, R², R³, R⁴, R⁵, and R⁶ of formula (V) is independently hydrogen, unsubstituted C1 to C40 hydrocarbyl, substituted C1 to C40 hydrocarbyl, or a functional group comprising at least one element of Group 13-17 of the periodic table of the elements; X⁻ of formula (V) is a monoatomic or polyatomic anion; R⁷ of formula (V) is an unsubstituted C1 to C40 hydrocarbyl or a substituted C1 to C40 hydrocarbyl; and when (a) R⁶ is methyl, (b) X⁻ is BF₄⁻ or CF₃CO₂⁻, (c) each of R¹, R², R³, and R⁴ are hydrogen, and (e) R⁵ is

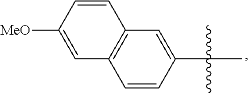 [SMILES: *c1ccc2cc(OC)ccc2c1]

then R⁷ is not —(CH₂)₆N₃ or

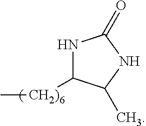

Clause D2. The composition of Clause D1, wherein each of R¹, R², R³, R⁴, and R⁵ of formula (V) is independently hydrogen, unsubstituted C1 to C20 hydrocarbyl, substituted C1 to C20 hydrocarbyl, or a functional group comprising at least one element of Group 13-17 of the periodic table of the elements.

Clause D3. The composition of Clause DI or D2, wherein: each of R¹, R², R³, and R⁴ of formula (V) is independently hydrogen, a C1 to C10 alkyl, or a C1 to C10 alkoxy; and/or R⁵ is an unsubstituted aryl or a substituted aryl.

Clause D4. The composition of any one of Clauses DI-D3, wherein: each of R¹ and R² of formula (V) is independently C1-C4 alkyl; each of R³ and R⁴ of formula (V) is hydrogen; and/or R⁵ of formula (V) is unsubstituted phenyl, substituted phenyl, unsubstituted naphthyl, or substituted naphthyl.

Clause D5. The composition of any one of Clauses DI-D4, wherein R⁵ of formula (V) is represented by formula (IIa):

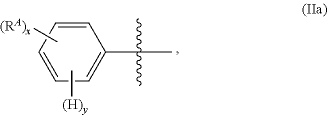

wherein: the wavy bond of formula (IIa) represents the connection to the pyridine ring; H of formula (IIa) is a hydrogen atom on the aromatic ring; R^{A} of formula (IIa) is an unsubstituted hydrocarbyl having from 1 to 20 carbon atoms, a substituted hydrocarbyl having from 1 to 20 carbon atoms; x of formula (IIa) is from 0 to 5, and when x is more than 1, each R^{A} group is the same or different; y of formula (IIa) is from 1 to 5; and x+y of formula (IIa) is 5.

Clause D6. The composition of Clause D5, wherein each R^{A} of formula (IIa) is independently an unsubstituted hydrocarbyl having from 1 to 4 carbon atoms or an alkoxy group having from 1 to 4 carbon atoms.

Clause D7. The composition of Clause D5, wherein each R^{A} of formula (IIa) is independently methyl, ethyl, methoxy, or ethoxy.

Clause D8. The composition of any one of Clauses D1-D4, wherein R⁵ of formula (V) is

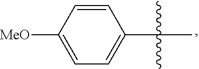 [SMILES: *c1ccc(OC)cc1]

the wavy bond representing the connection to the pyridine ring.

Clause D9. The composition of any one of Clauses D1-D4, wherein R⁵ of formula (V) is represented by formula (IIb):

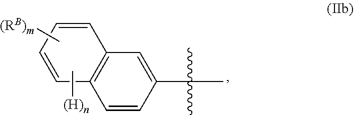

wherein: the wavy bond of formula (IIb) represents the connection to the pyridine ring; H of formula (IIb) is a hydrogen atom on the naphthyl ring; R^{B} of formula (IIb) is an unsubstituted hydrocarbyl having from 1 to 20 carbon atoms, a substituted hydrocarbyl having from 1 to 20 carbon atoms; m of formula (IIb) is from 0 to 7, and when m is more than 1, each R^{B} group is the same or different; n of formula (IIb) is from 0 to 7; and m+n of formula (IIb) is 7.

Clause D10. The composition of Clause D9, wherein each R^{B} of formula (IIb) is independently an unsubstituted hydrocarbyl having from 1 to 4 carbon atoms or an alkoxy group having from 1 to 4 carbon atoms.

Clause D11. The composition of Clause D9, wherein each R^{B} of formula (IIb) is independently methyl, ethyl, methoxy, or ethoxy.

Clause D12. The composition of any one of Clauses DI-D4, wherein R⁵ of formula (V) is

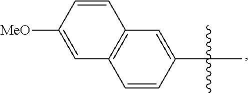 [SMILES: *c1ccc2cc(OC)ccc2c1]

the wavy bond representing the connection to the pyridine ring.

Clause D13. The composition of any one of Clauses D1-D12, wherein X⁻ of formula (V) is selected from the group consisting of F⁻, Cl⁻, Br⁻, and I⁻, BF₄⁻, CF₃CO₂⁻, BCl₄⁻, BBr₄⁻, BI₄⁻, NO₃⁻, NO₂⁻, ClO₄⁻, IO₃⁻, ClO₃⁻, BrO₃⁻, ClO₂⁻, OCl⁻, OBr⁻, CN⁻, OCN⁻, SCN⁻, KMnO₄⁻, HSO₄⁻, HSO₃⁻, RSO₃⁻, H₂PO₄⁻, OH⁻, CH₃CO₂⁻, HCO₂⁻, HCO₃⁻, H₃CC₆H₄SO₂⁻, F₃CSO₃⁻, CH₃SO₃⁻, C₇H₅O₂⁻, C₃H₅O₃⁻, (CH₃(CH₂)₃)₂HPO₄⁻, and (C₆H₅)₄B⁻.

Clause D14. The composition of any one of Clauses D1-D13, wherein X⁻ of formula (V) is selected from the group consisting of F⁻, Cl⁻, Br⁻, I⁻, BF₄⁻, CF₃CO₂⁻, PF₆⁻, H₃CC₆H₄SO₂⁻, SCN⁻, BCl₄⁻, BBr₄⁻, BI₄⁻, F₃CSO₃⁻, CH₃CO₂⁻, CH₃SO₃⁻, C₇H₅O₂⁻, C₃H₅O₃⁻, (CH₃(CH₂)₃)₂HPO₄⁻, and (C₆H₅)₄B⁻.

Clause D15. The composition of any one of Clauses DI-D14, wherein R⁷ of formula (V) is a substituted C1 to C40 hydrocarbyl comprising a sulfur atom.

Clause D16. The composition of any one of Clauses D1-D15, wherein the pyridinium salt of formula (V) is represented by formula (IVa), formula (IVb), formula (IVc), or formula (IVd):

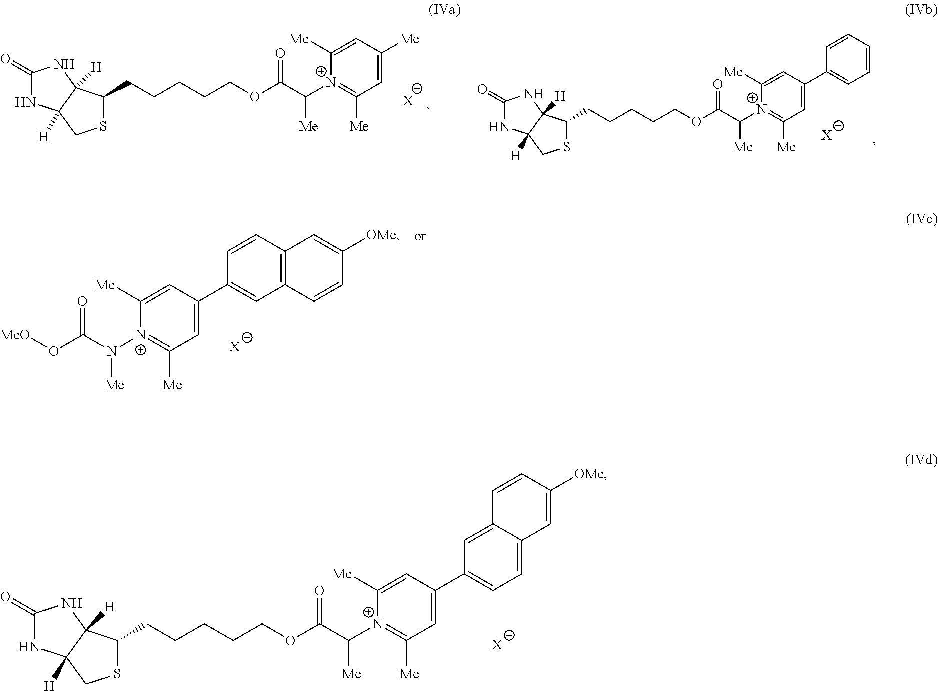

wherein: X⁻ of formulas (IVa)-(IVd) is a monoatomic or polyatomic anion.

Clause E1. A composition, comprising: a pyridinium salt represented by formula (I), an ion thereof, or combinations thereof:

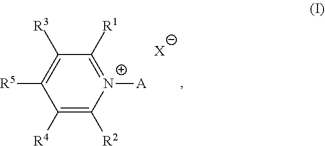

wherein: each of R¹, R², R³, R⁴, and R⁵ of formula (I) is independently hydrogen, unsubstituted C1 to C10 hydrocarbyl, substituted C1 to C10 hydrocarbyl, or a functional group comprising at least one element of Group 13-17 of the periodic table of the elements; X⁻ of formula (I) is selected from the group consisting of F⁻, Cl⁻, Br⁻, and I⁻, BF₄⁻, CF₃CO₂⁻, BCl₄⁻, BBr₄⁻, BI₄⁻, NO₃⁻, NO₂⁻, ClO₄⁻, IO₃⁻, ClO₃⁻, BrO₃⁻, ClO₂⁻, OCl⁻, OBr⁻, CN⁻,OCN⁻, SCN⁻, KMnO₄⁻, HSO₄⁻, HSO₃⁻, RSO₃⁻, H₂PO₄⁻, OH⁻, CH₃CO₂⁻, HCO₂⁻, HCO₃⁻, H₃CC₆H₄SO₂⁻, F₃CSO₃⁻, CH₃SO₃⁻, C₇H₅O₂⁻, C₃H₅O₃⁻, (CH₃(CH₂)₃)₂HPO₄⁻, and (C₆H₅)₄B⁻; and A of formula (I) is selected from the group consisting of formula (IIIb) and formula (IIIc):

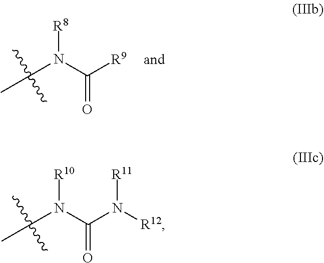

wherein: each of R⁸ and R⁹ of formula (IIIb) is independently hydrogen, unsubstituted C1 to C20 hydrocarbyl, or substituted C1 to C20 hydrocarbyl; and each of R¹⁰, R¹¹, and R¹² of formula (IIIc) is independently hydrogen, unsubstituted C1 to C20 hydrocarbyl, or substituted C1 to C20 hydrocarbyl.

Clause E2. The composition of Clause E1, wherein: each of R¹, R², R³, and R⁴ of formula (I) is independently hydrogen, a C1 to C10 alkyl, or a C1 to C10 alkoxy; and/or R⁵ is an unsubstituted aryl or a substituted aryl.

Clause F1. A method for modifying a tryptophan moiety, comprising:
forming a mixture comprising:
   a molecule containing a tryptophan moiety; and
   a composition comprising a pyridinium salt, an ion thereof, or a combination thereof; and
exposing the mixture to light to chemically modify the tryptophan moiety of the molecule, the pyridinium salt represented by formula (V):

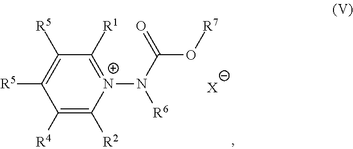

wherein: each of R¹, R², R³, R⁴, R⁵, and R⁶ of formula (V) is independently hydrogen, unsubstituted C1 to C40 hydrocarbyl, substituted C1 to C40 hydrocarbyl, or a functional group comprising at least one element of Group 13-17 of the periodic table of the elements; X⁻ of formula (V) is a monoatomic or polyatomic anion; and R⁷ of formula (V) is an unsubstituted C1 to C40 hydrocarbyl or a substituted C1 to C40 hydrocarbyl; and when (a) R⁶ is methyl, (b) X⁻ is BF₄⁻ or CF₃CO₂⁻, (c) each of R¹, R², R³, and R⁴ are hydrogen, and (d) R⁵ is

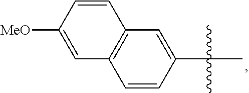 [SMILES: *c1ccc2cc(OC)ccc2c1]

then R⁷ is not —(CH₂)₆N₃ or

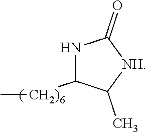

Clause F2. The method of Clause E2, wherein: each of R¹ and R² of formula (V) are C1 to C10 alkyl; R⁵ is a C1 to C10 alkyl, an unsubstituted aryl having from 5 to 20 carbon atoms, or a substituted aryl having from 5 to 20 carbon atoms; and/or R⁶ of formula (V) is C1 to C10 alkyl.

Clause G1. A method for modifying a tryptophan moiety, comprising: exposing a mixture comprising a composition of any one of Clauses A1-A15, DI-D16, and E1-E2 and a molecule containing a tryptophan residue to light to chemically modify the tryptophan moiety of the molecule.

Clause G2. The method of Clause G2, wherein the molecule is a biological molecule, a biologically-derived molecule, or a synthetic molecule.

All documents described herein are incorporated by reference herein, including any priority documents and/or testing procedures to the extent they are not inconsistent with this text. As is apparent from the foregoing general description and the specific embodiments, while forms of the embodiments have been illustrated and described, various modifications can be made without departing from the spirit and scope of the present disclosure. Accordingly, it is not intended that the present disclosure be limited thereby. Likewise, the term “comprising” is considered synonymous with the term “including.” Likewise whenever a composition, an element, a group of elements, or a method is preceded with the transitional phrase “comprising,” it is understood that we also contemplate the same composition, method, or group of elements with transitional phrases “consisting essentially of,” “consisting of,” “selected from the group of consisting of,” or “Is” preceding the recitation of the composition, element, elements, or method, and vice versa, such as the terms “comprising,” “consisting essentially of,” “consisting of” also include the product of the combinations of elements listed after the term.

As used herein, reference to an R group, alkyl, substituted alkyl, hydrocarbyl, substituted hydrocarbyl, aryl, substituted aryl, etc. without specifying a particular isomer (such as butyl) expressly discloses all isomers (such as n-butyl, iso-butyl, sec-butyl, and tert-butyl). For example, reference to an R group having 4 carbon atoms expressly discloses all isomers thereof. When a compound is described herein such that a particular isomer, enantiomer, diastereomer, or rotamer of the compound is not specified, for example, in a formula or in a chemical name, that description is intended to include each isomer and enantiomer of the compound described individual or in any combination.

In the foregoing, reference is made to embodiments of the disclosure. However, it should be understood that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the foregoing aspects, features, embodiments and advantages are merely illustrative and are not considered elements or limitations of the appended claims except where explicitly recited in a claim(s). Likewise, reference to “the disclosure” shall not be construed as a generalization of any inventive subject matter disclosed herein and shall not be considered to be an element or limitation of the appended claims except where explicitly recited in a claim(s).

For purposes of this present disclosure, and unless otherwise specified, all numerical values within the detailed description and the claims herein are modified by “about” or “approximately” the indicated value, and consider experimental error and variations that would be expected by a person having ordinary skill in the art. For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. For example, the recitation of the numerical range 1 to 5 includes the subranges 1 to 4, 1.5 to 4.5, 1 to 2, among other subranges. As another example, the recitation of the numerical ranges 1 to 5, such as 2 to 4, includes the subranges 1 to 4 and 2 to 5, among other subranges. Additionally, within a range includes every point or individual value between its end points even though not explicitly recited. For example, the recitation of the numerical range 1 to 5 includes the numbers 1, 1.5, 2, 2.75, 3, 3.80, 4, 5, among other numbers. Thus, every point or individual value may serve as its own lower or upper limit combined with any other point or individual value or any other lower or upper limit, to recite a range not explicitly recited.

As used herein, the indefinite article “a” or “an” shall mean “at least one” unless specified to the contrary or the context clearly indicates otherwise. For example, embodiments comprising “a pyridinium salt” include embodiments comprising one, two, or more pyridinium salts, unless specified to the contrary or the context clearly indicates only one pyridinium salt is included.

While the foregoing is directed to embodiments of the present disclosure, other and further embodiments of the disclosure may be devised without departing from the basic scope thereof, and the scope thereof is determined by the claims that follow.

1. A composition, comprising:
a pyridinium salt represented by formula (V), an ion thereof, or combinations thereof: 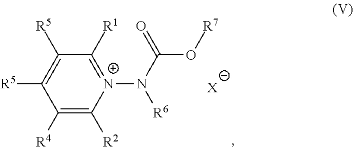
wherein:
each of R¹, R², R³, R⁴, R⁵, and R⁶ of formula (V) is independently hydrogen, unsubstituted C1 to C40 hydrocarbyl, substituted C1 to C40 hydrocarbyl, or a functional group comprising at least one element of Group 13-17 of the periodic table of the elements;
X⁻ of formula (V) is a monoatomic or polyatomic anion; and
R⁷ of formula (V) is an unsubstituted C1 to C40 hydrocarbyl or a substituted C1 to C40 hydrocarbyl; and
when (a) R⁶ is methyl, (b) X is BF₄⁻ or CF₃CO₂⁻, (c) each of R¹ and R² are methyl, (d) each of R³ and R⁴ are hydrogen, and e) R⁵ is 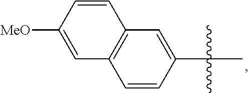 [SMILES: *c1ccc2cc(OC)ccc2c1]
then R⁷ is not —(CH₂)₆N₃ or

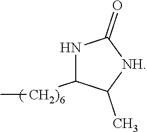

2. The composition of claim 1, wherein each of R¹, R², R³, R⁴, and R⁵ of formula (V) is independently hydrogen, unsubstituted C1 to C20 hydrocarbyl, substituted C1 to C20 hydrocarbyl, or a functional group comprising at least one element of Group 13-17 of the periodic table of the elements.

3. The composition of claim 1, wherein:
each of R¹, R², R³, and R⁴ of formula (V) is independently hydrogen, a C1 to C10 alkyl, or a C1 to C10 alkoxy; and
R⁵ is an unsubstituted aryl or a substituted aryl.

4. The composition of claim 1, wherein:
each of R¹ and R² of formula (V) is independently C1-C4 alkyl;
each of R³ and R⁴ of formula (V) is hydrogen; and
R⁵ of formula (V) is unsubstituted phenyl, substituted phenyl, unsubstituted naphthyl, or substituted naphthyl.

5. The composition of claim 1, wherein R⁵ of formula (V) is represented by formula (IIa): 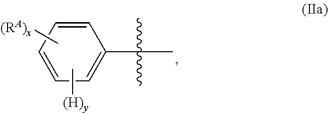
wherein:
the wavy bond of formula (IIa) represents a connection to the pyridine ring;
H of formula (IIa) is a hydrogen atom on the aromatic ring;
R^{A} of formula (IIa) is an unsubstituted hydrocarbyl having from 1 to 20 carbon atoms, a substituted hydrocarbyl having from 1 to 20 carbon atoms;
x of formula (IIa) is from 0 to 5, and when x is more than 1, each R^{A} group is the same or different;
y of formula (IIa) is from 1 to 5; and
x+y of formula (IIa) is 5.

6. The composition of claim 5, wherein each R^{A} of formula (IIa) is independently an unsubstituted hydrocarbyl having from 1 to 4 carbon atoms or an alkoxy group having from 1 to 4 carbon atoms.

7. The composition of claim 5, wherein each R^{A} of formula (IIa) is independently methyl, ethyl, methoxy, or ethoxy.

8. The composition of claim 1, wherein R⁵ of formula (V) is 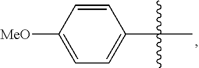 [SMILES: *c1ccc(OC)cc1]
the wavy bond of formula (V) representing a connection to the pyridine ring.

9. The composition of claim 1, wherein R⁵ of formula (V) is represented by formula (IIb): 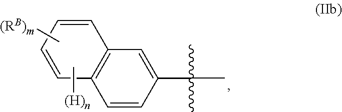
wherein:
the wavy bond of formula (IIb) represents a connection to the pyridine ring;
H of formula (IIb) is a hydrogen atom on the naphthyl ring;
R^{B} of formula (IIb) is an unsubstituted hydrocarbyl having from 1 to 20 carbon atoms, a substituted hydrocarbyl having from 1 to 20 carbon atoms;
m of formula (IIb) is from 0 to 7, and when m is more than 1, each R^{B} group is the same or different;
n of formula (IIb) is from 0 to 7; and
m+n of formula (IIb) is 7.

10. The composition of claim 9, wherein each R^{B} of formula (IIb) is independently an unsubstituted hydrocarbyl having from 1 to 4 carbon atoms or an alkoxy group having from 1 to 4 carbon atoms.

11. The composition of claim 9, wherein each R^{B} of formula (IIb) is independently methyl, ethyl, methoxy, or ethoxy.

12. The composition of claim 1, wherein R⁵ of formula (V) is 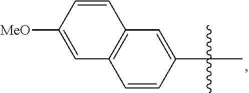 [SMILES: *c1ccc2cc(OC)ccc2c1]
the wavy bond of formula (V) representing a connection to the pyridine ring.

13. The composition of claim 1, wherein X⁻ of formula (V) is selected from the group consisting of F⁻, Cl⁻, Br⁻, and I⁻, BF₄⁻, CF₃CO₂⁻, BCl₄⁻, BBr₄⁻, BI₄⁻, NO₃⁻, NO₂⁻, ClO₄⁻, IO₃⁻, ClO₃⁻, BrO₃⁻, ClO₂⁻, OCl⁻, OBr⁻, CN, OCN⁻, SCN⁻, KMnO₄⁻, HSO₄⁻, HSO₃⁻, RSO₃⁻, H₂PO₄⁻, OH⁻, CH₃CO₂⁻, HCO₂⁻, HCO₃⁻, H₃CC₆H₄SO₂⁻, F₃CSO₃⁻, CH₃SO₃⁻, C₇H₅O₂⁻, C₃H₅O₃⁻, (CH₃(CH₂)₃)₂HPO₄⁻, and (C₆H₅)₄B⁻.

14. The composition of claim 1, wherein X⁻ of formula (V) is selected from the group consisting of F⁻, Cl⁻, Br⁻, I⁻, BF₄⁻, CF₃CO₂⁻, PF₆⁻, H₃CC₆H₄SO₂⁻, SCN⁻, BCl₄⁻, BBr₄⁻, BI₄⁻, F₃CSO₃⁻, CH₃CO₂⁻, CH₃SO₃⁻, C₇H₅O₂, C₃H₅O₃⁻, (CH₃(CH₂)₃)₂HPO₄⁻, and (C₆H₅)₄B⁻.

15. The composition of claim 1, wherein R⁷ of formula (V) is a substituted C1 to C40 hydrocarbyl comprising a sulfur atom.

16. The composition of claim 1, wherein the pyridinium salt of formula (V) is represented by formula (IVa), formula (IVb), formula (IVc), or formula (IVd): 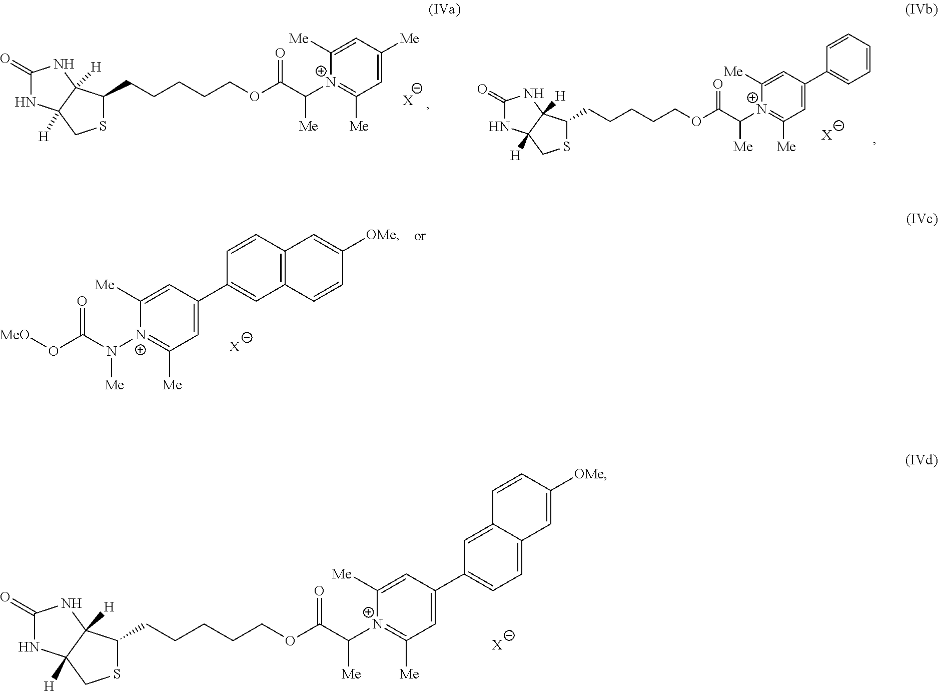
wherein:
X⁻ of formulas (IVa)-(IVd) is a monoatomic or polyatomic anion.

17. A composition, comprising:
a pyridinium salt represented by formula (I), an ion thereof, or combinations thereof: 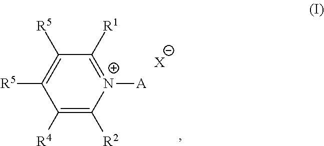
wherein:
each of R¹, R², R³, R⁴, and R⁵ of formula (I) is independently hydrogen, unsubstituted C1 to C10 hydrocarbyl, substituted C1 to C10 hydrocarbyl, or a functional group comprising at least one element of Group 13-17 of the periodic table of the elements;
X⁻ of formula (I) is selected from the group consisting of F⁻, Cl⁻, Br⁻, and I⁻, BF₄⁻, CF₃CO₂⁻, BCl₄⁻, BBr₄⁻, BI₄⁻, NO₃⁻, NO₂⁻, ClO₄⁻, IO₃⁻, ClO₃⁻, BrO₃⁻, ClO₂⁻, OCl⁻, OBr⁻, CN⁻, OCN⁻, SCN⁻, KMnO₄⁻, HSO₄⁻, HSO₃⁻, RSO₃⁻, H₂PO₄⁻, OH⁻, CH₃CO₂⁻, HCO₂⁻, HCO₃⁻, H₃CC₆H₄SO₂⁻, F₃CSO₃⁻, CH₃SO₃⁻, C₇H₅O₂⁻, C₃H₅O₃⁻, (CH₃(CH₂)₃)₂HPO₄⁻, and (C₆H₅)₄B⁻; and
A of formula (I) is selected from the group consisting of formula (IIIb) and formula (IIIc): 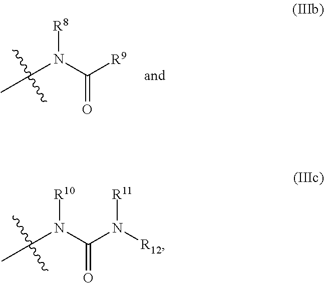
wherein:
each of R⁸ and R⁹ of formula (IIIb) is independently hydrogen, unsubstituted C1 to C20 hydrocarbyl, or substituted C1 to C20 hydrocarbyl; and
each of R¹⁰, R¹¹, and R¹² of formula (IIIc) is independently hydrogen, unsubstituted C1 to C20 hydrocarbyl, or substituted C1 to C20 hydrocarbyl.

18. The composition of claim 17, wherein:
each of R¹, R², R³, and R⁴ of formula (I) is independently hydrogen, a C1 to C10 alkyl, or a C1 to C10 alkoxy; and
R⁵ is an unsubstituted aryl or a substituted aryl.

19. A method for modifying a tryptophan moiety, comprising:
forming a mixture comprising:
a molecule containing a tryptophan moiety; and
a composition comprising a pyridinium salt, an ion thereof, or a combination thereof; and
exposing the mixture to light to chemically modify the tryptophan moiety of the molecule, the pyridinium salt represented by formula (V): 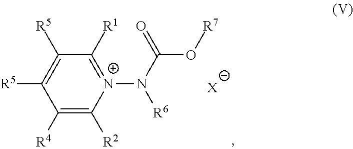
wherein:
each of R¹, R², R³, R⁴, R⁵, and R⁶ of formula (V) is independently hydrogen, unsubstituted C1 to C40 hydrocarbyl, substituted C1 to C40 hydrocarbyl, or a functional group comprising at least one element of Group 13-17 of the periodic table of the elements;
X⁻ of formula (V) is a monoatomic or polyatomic anion; and
R⁷ of formula (V) is an unsubstituted C1 to C40 hydrocarbyl or a substituted C1 to C40 hydrocarbyl; and
when (a) R⁶ is methyl, (b) X⁻ is BF₄⁻ or CF₃CO₂⁻, (c) each of R¹, R², R³, and R⁴ are hydrogen, and (d) R⁵ is 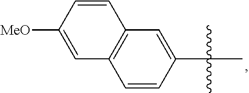 [SMILES: *c1ccc2cc(OC)ccc2c1]
then R⁷ is not —(CH₂)₆N₃ or

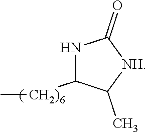

20. The method of claim 19, wherein:
each of R¹ and R² of formula (V) are C1 to C10 alkyl;
R⁵ is a C1 to C10 alkyl, an unsubstituted aryl having from 5 to 20 carbon atoms, or a substituted aryl having from 5 to 20 carbon atoms;
R⁶ of formula (V) is C1 to C10 alkyl;
the molecule is a biological molecule, a biologically-derived molecule, or a synthetic molecule; or
combinations thereof.
